SEQUENCE LISTING (111 sequences)

SEQ ID NO: 1 - protein, 214 aa, Artificial Sequence
  Synthetic Construct
DVQITQSPSSLSASVGDRVTITCRTSRSISQYLAWYQEKPGKTNKLLIYSGSTLQSGIPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, Artificial Sequence
  Synthetic Construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 374 aa, Artificial Sequence
  Synthetic Construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 4 - protein, 214 aa, Artificial Sequence
  Synthetic Construct
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 652 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPMAKKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSPMAKKGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 652 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 448 aa, Artificial Sequence
  Synthetic Construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 655 aa, Artificial Sequence
  Synthetic Construct
EVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGCGTKVEIKGGGGSGGSGGSSVHMPLGFLGPRQARVVNGGSGGSGGSGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 415 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPMAKKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSPMAKKGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 10 - protein, 415 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 11 - protein, 418 aa, Artificial Sequence
  Synthetic Construct
EVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGCGTKVEIKGGGGSGGSGGSSVHMPLGFLGPRQARVVNGGSGGSGGSGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 12 - protein, 282 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPMAKKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSPMAKKGGGGSGGGGSGGGGSGGGGSGGG

SEQ ID NO: 13 - protein, 133 aa, Homo Sapiens [sequence omitted]

SEQ ID NO: 14 - protein, 370 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 35 aa, Artificial Sequence
  Synthetic Construct
GGGGSGGGGSGPLGLWSQGGGGSGGGGSGGGGSGG

SEQ ID NO: 16 - protein, 8 aa, Artificial Sequence
  Synthetic Construct
RQARVVNG

SEQ ID NO: 17 - protein, 18 aa, Artificial Sequence
  Synthetic Construct
VHMPLGFLGPGRSRGSFP

SEQ ID NO: 18 - protein, 21 aa, Artificial Sequence
  Synthetic Construct
  misc_feature (9)..(13): Xaa can be any naturally occurring amino acid
RQARVVNGXXXXXVPLSLYSG

SEQ ID NO: 19 - protein, 16 aa, Artificial Sequence
  Synthetic Construct
RQARVVNGVPLSLYSG

SEQ ID NO: 20 - protein, 7 aa, Artificial Sequence
  Synthetic Construct
PLGLWSQ

SEQ ID NO: 21 - protein, 18 aa, Artificial Sequence
  Synthetic Construct
VHMPLGFLGPRQARVVNG

SEQ ID NO: 22 - protein, 6 aa, Artificial Sequence
  Synthetic Construct
FVGGTG

SEQ ID NO: 23 - protein, 8 aa, Artificial Sequence
  Synthetic Construct
KKAAPVNG

SEQ ID NO: 24 - protein, 8 aa, Artificial Sequence
  Synthetic Construct
PMAKKVNG

SEQ ID NO: 25 - protein, 8 aa, Artificial Sequence
  Synthetic Construct
QARAKVNG

SEQ ID NO: 26 - protein, 10 aa, Artificial Sequence
  Synthetic Construct
VHMPLGFLGP

SEQ ID NO: 27 - protein, 5 aa, Artificial Sequence
  Synthetic Construct
QARAK

SEQ ID NO: 28 - protein, 15 aa, Artificial Sequence
  Synthetic Construct
VHMPLGFLGPPMAKK

SEQ ID NO: 29 - protein, 5 aa, Artificial Sequence
  Synthetic Construct
KKAAP

SEQ ID NO: 30 - protein, 5 aa, Artificial Sequence
  Synthetic Construct
PMAKK

SEQ ID NO: 31 - protein, 8 aa, Artificial Sequence
  Synthetic Construct
YAARKGGI

SEQ ID NO: 32 - protein, 5 aa, Artificial Sequence
  Synthetic Construct
PQARK

SEQ ID NO: 33 - protein, 5 aa, Artificial Sequence
  Synthetic Construct
HQARK

SEQ ID NO: 34 - protein, 218 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 36 - protein, 651 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSPQARKGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 450 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 652 aa, Artificial Sequence; identical to SEQ ID NO: 6
  Synthetic Construct

SEQ ID NO: 39 - protein, 370 aa, Artificial Sequence; identical to SEQ ID NO: 14
  Synthetic Construct

SEQ ID NO: 40 - protein, 879 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 41 - protein, 882 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSGGGGSGGGGSGGGGSGGGGSGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 42 - protein, 597 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 43 - protein, 731 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 44 - protein, 443 aa, Artificial Sequence
  Synthetic Construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 45 - protein, 218 aa, Artificial Sequence
  Synthetic Construct
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - protein, 650 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSPQARKGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 47 - protein, 650 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 48 - protein, 368 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 49 - protein, 873 aa, Artificial Sequence
  Synthetic Construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 50 - protein, 873 aa, Artificial Sequence
  Synthetic Construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 51 - protein, 591 aa, Artificial Sequence
  Synthetic Construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 52 - protein, 218 aa, Artificial Sequence
  Synthetic Construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 443 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 54 - protein, 873 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 55 - protein, 873 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 56 - protein, 591 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 57 - protein, 725 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 58 - protein, 591 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 59 - protein, 725 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 60 - protein, 448 aa, Artificial Sequence; identical to SEQ ID NO: 2
  Synthetic Construct

SEQ ID NO: 61 - protein, 214 aa, Artificial Sequence; identical to SEQ ID NO: 4
  Synthetic Construct

SEQ ID NO: 62 - protein, 652 aa, Artificial Sequence; identical to SEQ ID NO: 5
  Synthetic Construct

SEQ ID NO: 63 - protein, 650 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPMAKKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSPMAKKGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 64 - protein, 218 aa, Artificial Sequence
  Synthetic Construct
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - protein, 607 aa, Artificial Sequence
  Synthetic Construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 66 - protein, 444 aa, Artificial Sequence
  Synthetic Construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 67 - protein, 656 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGYAARKGGIGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSYAARKGGIGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 68 - protein, 450 aa, Artificial Sequence
  Synthetic Construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 69 - protein, 689 aa, Artificial Sequence
  Synthetic Construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 215 aa, Artificial Sequence
  Synthetic Construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 71 - protein, 415 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSPQARKGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 72 - protein, 415 aa, Artificial Sequence; identical to SEQ ID NO: 10
  Synthetic Construct

SEQ ID NO: 73 - protein, 415 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 74 - protein, 418 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSGGGGSGGGGSGGGGSGGGGSGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 75 - protein, 282 aa, Artificial Sequence
  Synthetic Construct
GGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 76 - protein, 415 aa, Artificial Sequence; identical to SEQ ID NO: 71
  Synthetic Construct

SEQ ID NO: 77 - protein, 415 aa, Artificial Sequence; identical to SEQ ID NO: 10
  Synthetic Construct

SEQ ID NO: 78 - protein, 415 aa, Artificial Sequence; identical to SEQ ID NO: 73
  Synthetic Construct

SEQ ID NO: 79 - protein, 415 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 80 - protein, 415 aa, Artificial Sequence; identical to SEQ ID NO: 73
  Synthetic Construct

SEQ ID NO: 81 - protein, 415 aa, Artificial Sequence; identical to SEQ ID NO: 79
  Synthetic Construct

SEQ ID NO: 82 - protein, 282 aa, Artificial Sequence; identical to SEQ ID NO: 75
  Synthetic Construct

SEQ ID NO: 83 - protein, 282 aa, Artificial Sequence
  Synthetic Construct
GGGGSGGGGSGGGGSGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGGGSGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 84 - protein, 415 aa, Artificial Sequence; identical to SEQ ID NO: 9
  Synthetic Construct

SEQ ID NO: 85 - protein, 415 aa, Artificial Sequence; identical to SEQ ID NO: 9
  Synthetic Construct

SEQ ID NO: 86 - protein, 421 aa, Artificial Sequence
  Synthetic Construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGYAARKGGIGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSYAARKGGIGGGGSGGGGSGGGGSGGGGSGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 87 - protein, 111 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 88 - protein, 120 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 89 - protein, 111 aa, Artificial Sequence
  Synthetic Construct
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIK

SEQ ID NO: 90 - protein, 120 aa, Artificial Sequence
  Synthetic Construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSS

SEQ ID NO: 91 - protein, 111 aa, Artificial Sequence
  Synthetic Construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELK

SEQ ID NO: 92 - protein, 120 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 93 - protein, 107 aa, Artificial Sequence
  Synthetic Construct
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIK

SEQ ID NO: 94 - protein, 118 aa, Artificial Sequence [sequence omitted]

SEQ ID NO: 95 - protein, 109 aa, Artificial Sequence
  Synthetic Construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 96 - protein, 125 aa, Artificial Sequence
  Synthetic Construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 97 - protein, 109 aa, Artificial Sequence; identical to SEQ ID NO: 95
  Synthetic Construct

SEQ ID NO: 98 - protein, 120 aa, Artificial Sequence
  Synthetic Construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSS

SEQ ID NO: 99 - protein, 215 aa, Artificial Sequence
  Synthetic Construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 100 - protein, 870 aa, Artificial Sequence
  Synthetic Construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGGGGSGGGGSGGGGSGGGGSGGGGSGGGGGHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRPGGGGSGGGGSGGGPQARKGGGGGGSGGGGGQVQLQESGPGLVQASQPLSLTCTVSGFSLTTNSVHWIRQPAGKCLEWMGAVWTDGTTDYNSALKSRLTISRDTSKSQVFLEMSSLQTEDVATYYCAREHVYYGLVGWGQGTLVTVSAGGGGSGGGGSGGGGSNIVMTQSPKSMSISVGDRVTMNCKASQNVGNNIAWYQQNPGQSPKLLIYYASNRFTGVPDRFTGGGYGTDFTLTINNVQTEDAASYYCQRIFNAPNTFGCGTKLELK

SEQ ID NO: 101 - protein, 30 aa, Artificial Sequence
  Synthetic Construct
GGGGSGGGGSGGGGSGGGGSGGGGSGGGGG

SEQ ID NO: 102 - protein, 30 aa, Artificial Sequence
  Synthetic Construct
GGGGSGGGGSGGGPQARKGGGGGGSGGGGG

SEQ ID NO: 103 - protein, 131 aa, Mus musculus
HGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRP

SEQ ID NO: 104 - protein, 239 aa, Artificial Sequence
  Synthetic Construct
QVQLQESGPGLVQASQPLSLTCTVSGFSLTTNSVHWIRQPAGKCLEWMGAVWTDGTTDYNSALKSRLTISRDTSKSQVFLEMSSLQTEDVATYYCAREHVYYGLVGWGQGTLVTVSAGGGGSGGGGSGGGGSNIVMTQSPKSMSISVGDRVTMNCKASQNVGNNIAWYQQNPGQSPKLLIYYASNRFTGVPDRFTGGGYGTDFTLTINNVQTEDAASYYCQRIFNAPNTFGCGTKLELK

SEQ ID NO: 105 - protein, 215 aa, Artificial Sequence; identical to SEQ ID NO: 70
  Synthetic Construct

SEQ ID NO: 106 - protein, 450 aa, Artificial Sequence
  Synthetic Construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 971 aa, Artificial Sequence
  Synthetic Construct
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKSGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 971 aa, Artificial Sequence
  Synthetic Construct
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKSGGGSGGGGSHQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 971 aa, Artificial Sequence
  Synthetic Construct
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKSGGGSGGGGSPQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 33 aa, Artificial Sequence
  Synthetic Construct
GGGGSGGGGSPQARKGGGGSGGGGSGGGGSGGS

SEQ ID NO: 111 - protein, 33 aa, Artificial Sequence
  Synthetic Construct
GGGGSGGGGSHQARKGGGGSGGGGSGGGGSGGS

(19) United States
(12) Patent Application Publication
(10) Pub. No.: US 2024/0376171 A1
(43) Pub. Date: Nov. 14, 2024

(54) PROTEASE-ACTIVATED POLYPEPTIDES

(71) Applicant: Hoffmann-La Roche Inc., Little Falls, NJ (US)

(72) Inventors: Samuele CALABRO, Schlieren (CH); Peter BRUENKER, Hittnau (CH); Linda FAHRNI, Fällanden (CH); Anne FREIMOSER-GRUNDSCHOBER, Zürich (CH); Martina GEIGER, Obfelden (CH); Ralf HOSSE, Mettmenstetten (CH); Christian KLEIN, Bonstetten (CH); Ekkehard MOESSNER, Kreuzlingen (CH); Evelyn SAUER, Ehrendingen (CH); Pablo UMAÑA, Wollerau (CH); Inja WALDHAUER, Zürich (CH)

(73) Assignee: Hoffmann-La Roche Inc., Little Falls, NJ (US)

(21) Appl. No.: 18/553,504

(22) PCT Filed: Mar. 28, 2022
(86) PCT No.: PCT/EP2022/058039

§ 371 Date: Sep. 29, 2023

(30) Foreign Application Priority Data
Mar. 30, 2021 (EP) 21165900.8

(51) Int. Cl. C07K 14/55; A61K 38/00; C07K 16/28

(52) U.S. Cl. CPC C07K 14/55; C07K 16/2809; C07K 16/2815; C07K 16/2818; A61K 38/00; C07K 2317/622; C07K 2319/30; C07K 2319/50

(57) ABSTRACT

The present invention generally relates to novel isolated polypeptides and immunoconjugates and their uses. The polypeptides and immunoconjugates comprise at least one protease recognitions sequence, which is a substrate for matriptase.

### FIELD OF THE INVENTION

The present invention generally relates to novel protease-activated polypeptides, particularly interleukin-2 (IL-2) polypeptides. More particularly, the invention concerns protease-activated IL-2 polypeptides that exhibit improved properties for use as immunotherapeutic agents. In addition, the invention relates to protease-activated IL-2 polypeptides or immunoconjugates, polynucleotides, vectors and host cells comprising such vectors or polynucleotide molecules. The invention further relates to methods for producing the protease-activated IL-2 polypeptides or immunoconjugates, pharmaceutical compositions comprising the same, and uses thereof.

### BACKGROUND

The selective destruction of an individual target cell or a specific target cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. In this regard, conjugates designed to bind to a surface antigen on target cells and comprising interleukin-2 (IL-2) variants are supposed to activate nearby T effector cells and NK cells. The simultaneous binding of such an conjugate to its target and the interleukin-2 receptor will cause activation of T effector cells and NK cells in the proximity of the target (in trans) or when the target is expressed on the T effector cell and NK cell, this cell is activated upon binding (in cis).

Interleukin-2 (IL-2), also known as T cell growth factor (TCGF), is a 15.5 kDa globular glycoprotein playing a central role in lymphocyte generation, survival and homeostasis. It has a length of 133 amino acids and consists of four antiparallel, amphiphatic α-helices that form a quaternary structure indispensable of its function (Smith, Science 240, 1169-76 (1988); Bazan, Science 257, 410-413 (1992)). Sequences of IL-2 from different species are found under NCBI RefSeq Nos. NP000577 (human), NP032392 (mouse), NP446288 (rat) or NP517425 (chimpanzee).

IL-2 mediates its action by binding to IL-2 receptors (IL-2R), which consist of up to three individual subunits, the different association of which can produce receptor forms that differ in their affinity to IL-2. Association of the α (CD25), β (CD122), and γ (γ_{c}, CD132) subunits results in a trimeric, high-affinity receptor for IL-2. Dimeric IL-2 receptor consisting of the β and γ subunits is termed intermediate-affinity IL-2R. The α subunit forms the monomeric low affinity IL-2 receptor. Although the dimeric intermediate-affinity IL-2 receptor binds IL-2 with approximately 100-fold lower affinity than the trimeric high-affinity receptor, both the dimeric and the trimeric IL-2 receptor variants are able to transmit signal upon IL-2 binding (Minami et al., Annu Rev Immunol 11, 245-268 (1993)). Hence, the α-subunit, CD25, is not essential for IL-2 signalling. It confers high-affinity binding to its receptor, whereas the β subunit, CD122, and the γ-subunit are crucial for signal transduction (Krieg et al., Proc Natl Acad Sci 107, 11906-11 (2010)). Trimeric IL-2 receptors including CD25 are expressed by (resting) CD4⁺ forkhead box P3 (FoxP3)⁺ regulatory T (T_{reg}) cells. They are also transiently induced on conventional activated T cells, whereas in the resting state these cells express only dimeric IL-2 receptors. T_{reg} cells consistently express the highest level of CD25 in vivo (Fontenot et al., Nature Immunol 6, 1142-51 (2005)).

IL-2 is synthesized mainly by activated T-cells, in particular CD4⁺ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells (reviewed e.g. in Waldmann, Nat Rev Immunol 6, 595-601 (2009); Olejniczak and Kasprzak, Med Sci Monit 14, RA179-89 (2008); Malek, Annu Rev Immunol 26, 453-79 (2008)).

Its ability to expand lymphocyte populations in vivo and to increase the effector functions of these cells confers antitumor effects to IL-2, making IL-2 immunotherapy an attractive treatment option for certain metastatic cancers. Consequently, high-dose IL-2 treatment has been approved for use in patients with metastatic renal-cell carcinoma and malignant melanoma.

However, IL-2 has a dual function in the immune response in that it not only mediates expansion and activity of effector cells, but also is crucially involved in maintaining peripheral immune tolerance.

A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD3 complex, the expression of Fas ligand (FasL) and/or tumor necrosis factor (TNF) is induced, making the cells susceptible for Fas-mediated apoptosis. This process is IL-2 dependent (Lenardo, Nature 353, 858-61 (1991)) and mediated via STAT5. By the process of AICD in T lymphocytes tolerance cannot only be established to self-antigens, but also to persistent antigens that are clearly not part of the host's makeup, such as tumor antigens.

Moreover, IL-2 is also involved in the maintenance of peripheral CD4″ CD25″ regulatory T (T_{reg}) cells (Fontenot et al., Nature Immunol 6, 1142-51 (2005); D'Cruz and Klein, Nature Immunol 6, 1152-59 (2005); Maloy and Powrie, Nature Immunol 6, 1171-72 (2005), which are also known as suppressor T cells. They suppress effector T cells from destroying their (self-) target, either through cell-cell contact by inhibiting T cell help and activation, or through release of immunosuppressive cytokines such as IL-10 or TGF-β. Depletion of T_{reg} cells was shown to enhance IL-2 induced anti-tumor immunity (Imai et al., Cancer Sci 98, 416-23 (2007)).

Therefore, IL-2 is not optimal for inhibiting tumor growth, because in the presence of IL-2 either the CTLs generated might recognize the tumor as self and undergo AICD or the immune response might be inhibited by IL-2 dependent T_{reg} cells.

A further concern in relation to IL-2 immunotherapy are the side effects produced by recombinant human IL-2 treatment. Patients receiving high-dose IL-2 treatment frequently experience severe cardiovascular, pulmonary, renal, hepatic, gastrointestinal, neurological, cutaneous, haematological and systemic adverse events, which require intensive monitoring and in-patient management. The majority of these side effects can be explained by the development of so-called vascular (or capillary) leak syndrome (VLS), a pathological increase in vascular permeability leading to fluid extravasation in multiple organs (causing e.g. pulmonary and cutaneous edema and liver cell damage) and intravascular fluid depletion (causing a drop in blood pressure and compensatory increase in heart rate). There is no treatment of VLS other than withdrawal of IL-2. Low-dose IL-2 regimens have been tested in patients to avoid VLS, however, at the expense of suboptimal therapeutic results. VLS was believed to be caused by the release of proinflammatory cytokines, such as tumor necrosis factor (TNF)-α from IL-2-activated NK cells, however it has recently been shown that IL-2-induced pulmonary edema resulted from direct binding of IL-2 to lung endothelial cells, which expressed low to intermediate levels of functional αβγ IL-2 receptors (Krieg et al., Proc Nat Acad Sci USA 107, 11906-11 (2010)).

Several approaches have been taken to overcome these problems associated with IL-2 immunotherapy. For example, it has been found that the combination of IL-2 with certain anti-IL-2 monoclonal antibodies enhances treatment effects of IL-2 in vivo (Kamimura et al., J Immunol 177, 306-14 (2006); Boyman et al., Science 311, 1924-27 (2006)). In an alternative approach, IL-2 has been mutated in various ways to reduce its toxicity and/or increase its efficacy. Hu et al. (Blood 101, 4853-4861 (2003), US Pat. Publ. No. 2003/0124678) have substituted the arginine residue in position 38 of IL-2 by tryptophan to eliminate IL-2's vasopermeability activity. Shanafelt et al. (Nature Biotechnol 18, 1197-1202 (2000)) have mutated asparagine 88 to arginine to enhance selectivity for T cells over NK cells. Heaton et al. (Cancer Res 53, 2597-602 (1993); U.S. Pat. No. 5,229,109) have introduced two mutations, Arg38Ala and Phe42Lys, to reduce the secretion of proinflammatory cytokines from NK cells. Gillies et al. (US Pat. Publ. No. 2007/0036752) have substituted three residues of IL-2 (Asp20Thr, Asn88Arg, and Gln126Asp) that contribute to affinity for the intermediate-affinity IL-2 receptor to reduce VLS. Gillies et al. (WO 2008/0034473) have also mutated the interface of IL-2 with CD25 by amino acid substitution Arg38Trp and Phe42Lys to reduce interaction with CD25 and activation of T_{reg} cells for enhancing efficacy. To the same aim, Wittrup et al. (WO 2009/061853) have produced IL-2 mutants that have enhanced affinity to CD25, but do not activate the receptor, thus act as antagonists. The mutations introduced were aimed at disrupting the interaction with the β- and/or γ-subunit of the receptor.

A particular mutant IL-2 polypeptide, designed to overcome the above-mentioned problems associated with IL-2 immunotherapy (toxicity caused by the induction of VLS, tumor tolerance caused by the induction of AICD, and immunosuppression caused by activation of Treg cells), is described in WO 2012/107417. Substitution of the phenylalanine residue at position 42 by alanine, the tyrosine residue at position 45 by alanine and the leucine residue at position 72 of IL-2 by glycine essentially abolishes binding of this mutant IL-2 polypeptide to the α-subunit of the IL-2 receptor (CD25).

However, none of the known IL-2 mutants was shown to overcome all of the above-mentioned problems associated with IL-2 immunotherapy, namely toxicity caused by the induction of VLS, tumor tolerance caused by the induction of AICD, and immunosuppression caused by activation of T_{reg} cells.

Further to the above-mentioned approaches, IL-2 immunotherapy may be improved by selectively targeting IL-2 to tumors, e.g. in the form of immunoconjugates comprising an antibody that binds to an antigen expressed on tumor cells or that binds to effector cells in the tumor environment. Several such immunoconjugates have been described (see e.g. Ko et al., J Immunother (2004) 27, 232-239; Klein et al., Oncoimmunology (2017) 6 (3), e1277306; WO 2018/184964 A1).

Given the clinical success and unprecedented efficacy of PD-1/PD-L1 check-point inhibitors there remains a major medical need to increase the response rate and duration in patients with pre-existing T cell immunity further. Recent reports have demonstrated that two populations of tumor-specific CD8 T cells are targeted by PD-1 antibodies: the exhausted TILs and, their newly described TCF1+ precursor with stem-like properties, TResource cells. Of the two the latter correlates with a favorable disease prognosis and response to anti-PD-1 therapy. Cytokines, like interleukin-2, have also been described to induce the proliferation/differentiation of the TResource cells towards functional effector T cells.

IL-2 has been the first effective cancer immunotherapy used to treat metastatic melanoma and renal cell carcinoma. Unfortunately, IL-2, at high concentrations, is toxic by inducing vascular leak syndrome (VLS) and detrimentally expands regulatory T cells and induces activation induced cell death due to binding to CD25. In order to overcome these limitations of wildtype IL2/Proleukin, IL-2v variants with abolished CD25 binding have been described. However, due to the mechanism of IL-2 signaling through the heterodimeric intermediate affinity IL-2Rbg complex IL2v and other IL2 variants automatically activate IL-2R signaling as soon as they encounter the IL-2R and as a consequence mediate unspecific and peripheral immune cell activation outside of the tumor in blood, the vasculature and lymphoid tissues resulting in dose limiting toxicities. As a consequence, it is not possible to administer to a patients as much IL-2 or IL2v as desired in order to achieve the maximal therapeutic benefit.

Taken together, by targeting PD1-IL2v in cis to PD-1+ T cells a stronger therapeutic effect of PD1-IL2v can be achieved. In fact, cis-targeting of PD1-IL2v to appropriate antigen specific T-cell subsets, together with PD-1/-L1 inhibition is a better way to exploit endogenous immunity therapeutically and one of the strongest immunomodulatory pathway known for unleashing endogenous immunity for cancer immunotherapy. However, due to the possibility of the IL2v moiety to trigger IL-2R signaling in the periphery also for PD1-IL2v not the maximally desired dose can be administered due to the peripheral non-tumor specific IL-2R activation. Thus, the therapeutic index is believed to remain narrow with an anticipated MTD with a flat dose of >10-30 mg in man which may limit utilizing full pathway potential. CD8 T cells could be targeted instead, as well as other T cell targets.

Therefore, it is critical to generate next generations of IL-2 molecules cis-targeted to antigen-experienced T cells but with wider therapeutic index.

Serine proteases (e.g. matriptase), cysteine proteases (e.g. cathepsin S) and matrix metalloproteinases (e.g. MMP-2 and MMP-9) are overexpressed in several cancer types (Duffy, M. J. Proteases as prognostic markers in cancer. Clin. Cancer Res. 2, 613-618 (1996)). Matriptase, matrix metalloproteinase 2 (MMP-2, gelatinase A) and matrix metalloproteinase 9 (MMP-9, gelatinase B) are overexpressed e.g. in breast- and ovarian carcinoma (McGowan, P. M. & Duffy, M. J. Matrix metalloproteinase expression and outcome in patients with breast cancer: analysis of a published database. Ann. Oncol. 19, 1566-1572 (2008)). MMP-2 and MMP-9 activity was detected in cervical, breast and ovarian carcinoma and ascites of patients with epithelial ovarian cancer (EOC) but not in the serum of these patients (Demeter, A. et al. Molecular prognostic markers in recurrent and in non-recurrent epithelial ovarian cancer. Anticancer Res. 25, 2885-2889 (2005)). While matriptase can be detected in normal epithelial cells, matriptase activity is mainly detected in cancer (LeBeau, A. M. et al. Imaging a functional tumorigenic biomarker in the transformed epithelium. Proc. Natl. Acad. Sci. USA 110, 93-98 (2013)).

Although current immunotherapies directed against the PD1/PDL1 axis have shown unprecedented efficacy in several cancer indications, there is a substantial portion of patients who do not respond to the treatment or relapse, while other tumor-types remain largely refractory to these therapies. Therefore, there is a clear, high unmet need of a considerable cancer patient population in patients that have some kind of pre-existing T cell immune response. Examples for indications where PD1 antagonism has resulted in objective responses are e.g. advanced or metastatic melanoma, Merkel cell carcinoma, NSCLC, SCLC, RCC, gastric cancer, hepatocellular cancer, head and neck carcinoma, breast cancer, ovarian cancer, mismatch repair deficient versus sufficient CRC and haematological malignancies like DLBCL and PMBCL after autologous stem cell transplant and HL (Editiorial: PD-Loma: a cancer entity with a shared sensitivity to the PD-1/PD-L1 pathway blockade, British Journal of Cancer (2019) 120:3-5; https://doi.org/10.1038/s41416-018-0294-4).

The task of generating IL-2 variants and conjugates suitable for treatment provides several technical challenges related to efficacy, toxicity, applicability and produceability that have to be met. In instances where the conjugate targets an antigen on a target cell, e.g., a cancer cell, that is also expressed in non-target tissue, toxicity can occur. Thus there remains a need in the art to further enhance the therapeutic usefulness of IL-2 polypeptides.

### SUMMARY OF THE INVENTION

The present invention is based, in part, on the recognition that tumor environments (TME) highly express proteases compared to normal tissue and that a masked therapeutic agent, preferably protease-activatable interleukin-2 or protease-activatable interferon-γ or a protease-activatable T cell engager, has a reduced or abolished systemic activity and full activity in the tumor environment upon activation by proteases.

Accordingly, a first aspect of the invention provides an isolated polypeptide comprising a protease recognition site, wherein the protease recognition site is a substrate for matriptase and comprises or consists of the sequence PQARK according to SEQ ID NO: 32 or HQARK according to SEQ ID NO: 33. In one embodiment, the isolated polypeptide comprises one or more unstructured linker comprising the protease recognition site. In one embodiment, the one ore more unstructured linker does not exhibit a secondary structure. In one embodiment, the protease recognition site is part of a cleavable moiety (CM), preferably comprising one of the sequences selected from the group consisting of SEQ ID NOs 71, 73, 75, 76, 78, 80, 82. In one embodiment, the isolated polypeptide comprises at least one moiety (M) selected from the group consisting of a moiety that is located amino (N) terminally to the CM (MN), a moiety that is located carboxyl (C) terminally to the CM (MC), and combinations thereof, and wherein the MN or MC is selected from the group consisting of an antibody or antigen binding fragment thereof (AB), a therapeutic agent, an antineoplastic agent, a toxic agent, a drug, and a detectable label. In one embodiment, the isolated polypeptide comprises a sequence selected from the group consisting of GGGGSGGGGSGGGPQARKGGGGGGSGGGGG according to SEQ ID NO: 102, GGGGSGGGGSPQARKGGGGSGGGGSGGGGSGGS according to SEQ ID NO: 110 and GGGGSGGGGSHQARKGGGGSGGGGSGGGGSGGS according to SEQ ID NO: 111.

The invention further provides the use of a protease recognition site, wherein the protease recognition site is PQARK according to SEQ ID NO: 32 or HQARK according to SEQ ID NO: 33, wherein the protease recognition site is present in a therapeutic agent. In one embodiment, the therapeutic agent is an isolated polypeptide. In one embodiment, the therapeutic agent is a cancer treatment.

The invention further provides for the use of an isolated polypeptide as disclosed herein in a pharmaceutical composition.

The invention further provides one or more isolated polynucleotides encoding the isolated polypeptide as disclosed herein, one or more expression vectors comprising the one or more polynucleotides as disclosed herein, and one or more host cells comprising the one or more polynucleotides as disclosed herein or the one or more expression vector disclosed herein.

Also provided is a method of producing a polypeptide, comprising culturing the host cell as disclosed herein under conditions suitable for the expression of the polypeptide.

Also provided is an isolated polypeptide produced by the method as disclosed herein. Also provided is a pharmaceutical composition comprising the isolated polypeptide as disclosed herein and a pharmaceutically acceptable carrier. In particular, the invention encompasses the isolated polypeptide for use in the treatment of a disease in an individual in need thereof. In a particular embodiment, said disease is cancer. In a particular embodiment, the individual is a human.

Also encompassed by the invention is the use of the isolated polypeptide as disclosed herein for the manufacture of a medicament for treating a disease in an individual in need thereof.

The invention further provides a method of treating a disease in an individual, comprising administering to said individual a therapeutically effective amount of a composition comprising the isolated polypeptide as disclosed herein in a pharmaceutically acceptable form. The disease preferably is cancer.

The invention provides a protease-activatable interleukin-2 (IL-2) polypeptide comprising (i) an IL-2 polypeptide, (ii) a masking moiety and (iii) a linker comprising a first protease cleavage site, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site. In one embodiment, the masking moiety is covalently attached to the amino-terminus or the carboxy-terminus of the IL-2 polypeptide through the linker. In one embodiment, the masking moiety is an IL-2 antagonist. In one embodiment, the masking moiety is an IL-2 antibody or an IL-2 receptor subunit. In one embodiment, the IL-2 antibody comprises a Fab molecule. In one embodiment, the masking moiety is derived from MT204. In one embodiment, the masking moiety is MT204. The MT204 antibody is disclosed e.g. in Volkland et al., Molecular Immunology 44 (2007) 1743-1753, and PCT-publication WO 2006/128690 A1. In one embodiment, the Fab molecule is a single-chain Fab molecule. In one embodiment, the second protease cleavage site is located between the variable domain of the heavy chain (VH) and the variable domain of the light chain (VL) of the Fab. In one embodiment, the first protease cleavage site and the second protease cleavage site each comprise at least one protease recognition sequence. In one embodiment, the protease recognition sequence of the first protease cleavage site and/or the protease recognition sequence of the second protease cleavage site (recognition sequence) is selected from the group consisting of: (a) RQARVVNG (SEQ ID NO:16); (b) VHMPLGFLGPGRSRGSFP (SEQ ID NO:17); (c) RQARVVNGXXXXXVPLSLYSG (SEQ ID NO:18), wherein X is any amino acid; (d) RQARVVNGVPLSLYSG (SEQ ID NO:19); (e) PLGLWSQ (SEQ ID NO:20); (f) VHMPLGFLGPRQARVVNG (SEQ ID NO:21); (g) FVGGTG (SEQ ID NO:22); (h) KKAAPVNG (SEQ ID NO:23); (i) PMAKKVNG (SEQ ID NO:24); (j) QARAKVNG (SEQ ID NO: 25); (k) VHMPLGFLGP (SEQ ID NO:26); (l) QARAK (SEQ ID NO:27); (m) VHMPLGFLGPPMAKK (SEQ ID NO:28); (n) KKAAP (SEQ ID NO:29); (o) PMAKK (SEQ ID NO: 30); (p) YAARKGGI (SEQ ID NO:31); (q) PQARK (SEQ ID NO:32); and (r) HQARK (SEQ ID NO:33).

In one embodiment, the protease recognition sequence of the first protease cleavage site is different from the protease recognition sequence of the second protease cleavage site. In one embodiment, the protease recognition sequence of the first protease cleavage site is the same as the protease recognition sequence of the second protease cleavage site.

In one embodiment, the protease recognition sequence of the first protease cleavage site and/or the protease recognition sequence of the second protease cleavage site is selected from the group consisting of PMAKK (SEQ ID NO:30), PQARK (SEQ ID NO: 32) or HQARK (SEQ ID NO: 33). In one embodiment, the protease recognition sequence of the first protease cleavage site is selected from the group consisting of PMAKK (SEQ ID NO:30), PQARK (SEQ ID NO: 32) or HQARK (SEQ ID NO: 33). In one embodiment, the protease recognition sequence of the second protease cleavage site is selected from the group consisting of PMAKK (SEQ ID NO:30), PQARK (SEQ ID NO: 32) or HQARK (SEQ ID NO: 33). In one embodiment, the protease recognition sequence of the first protease cleavage site and the protease recognition sequence of the second protease cleavage site is selected from the group consisting of PMAKK (SEQ ID NO: 30), PQARK (SEQ ID NO: 32) or HQARK (SEQ ID NO: 33).

In one embodiment, the protease recognition sequence of the first protease cleavage site and/or the protease recognition sequence of the second protease cleavage site is PMAKK (SEQ ID NO: 30. In one embodiment, the protease recognition sequence of the first protease cleavage site is PMAKK (SEQ ID NO:30). In one embodiment, the protease recognition sequence of the second protease cleavage site is PMAKK (SEQ ID NO:30). In one embodiment, the protease recognition sequence of the first protease cleavage site and the protease recognition sequence of the second protease cleavage site is PMAKK (SEQ ID NO:30).

In one embodiment, the protease recognition sequence of the first protease cleavage site and/or the protease recognition sequence of the second protease cleavage site is PQARK (SEQ ID NO: 32). In one embodiment, the protease recognition sequence of the first protease cleavage site is PQARK (SEQ ID NO: 32). In one embodiment, the protease recognition sequence of the second protease cleavage site is PQARK (SEQ ID NO: 32). In one embodiment, the protease recognition sequence of the first protease cleavage site and the protease recognition sequence of the second protease cleavage site is PQARK (SEQ ID NO: 32).

In one embodiment, the protease recognition sequence of the first protease cleavage site and/or the protease recognition sequence of the second protease cleavage site is HQARK (SEQ ID NO: 33). In one embodiment, the protease recognition sequence of the first protease cleavage site is HQARK (SEQ ID NO: 33). In one embodiment, the protease recognition sequence of the second protease cleavage site is HQARK (SEQ ID NO: 33). In one embodiment, the protease recognition sequence of the first protease cleavage site and the protease recognition sequence of the second protease cleavage site is HQARK (SEQ ID NO: 33).

In one embodiment, the IL-2 polypeptide is a wild-type IL-2, preferably a human IL-2 according to SEQ ID NO: 13, or a mutant IL-2 polypeptide. In one embodiment, the mutant IL-2 polypeptide comprises any amino acid substitution selected from the group T3A, F42A, Y45A, L72G, C125A of human IL-2 according to SEQ ID NO: 13. In one embodiment, the mutant IL-2 polypeptide comprises the amino acid substitutions F42A, Y45A and L72G of human IL-2 according to SEQ ID NO:13. In one embodiment, the mutant IL-2 polypeptide comprises the amino acid substitutions T3A, F42A, Y45A, L72G and C125A of human IL-2 according to SEQ ID NO: 13.

In one embodiment, the masking moiety and the linker comprise the amino acid sequence of SEQ ID NO: 12. In one embodiment, the protease activatable IL-2 polypeptide comprises the amino acid sequence of SEQ ID NO: 9.

In one embodiment, the IL-2 polypeptide is further attached to a non-IL-2 moiety. In one embodiment, the IL-2 polypeptide shares a carboxy-terminal peptide bond with the masking moiety and an amino-terminal peptide bond with the non-IL-2 moiety or wherein said IL-2 polypeptide shares a amino-terminal peptide bond with the masking moiety and an carboxy-terminal peptide bond with the non-IL-2 moiety. In one embodiment, the non-IL-2 moiety is an antigen binding moiety or an effector cell binding moiety.

In a further aspect, the invention provides an immunoconjugate comprising a protease-activatable IL-2 polypeptide as described herein and an antigen binding moiety and/or an effector cell binding moiety. In one embodiment, said protease-activatable IL-2 polypeptide shares an amino- or carboxy-terminal peptide bond with the antigen binding moiety or the effector cell binding moiety. In one embodiment, said immunoconjugate comprises a first and a second antigen binding moiety or a first and a second effector cell antigen binding moiety or an antigen binding moiety and an effector cell binding moiety. In one embodiment, (i) the protease-activatable IL-2 polypeptide shares an amino- or carboxy-terminal peptide bond with said first antigen binding moiety and said second antigen binding moiety shares an amino- or carboxy-terminal peptide bond with either a) said protease-activatable IL-2 polypeptide or b) said first antigen binding moiety; (ii) the protease-activatable IL-2 polypeptide shares an amino- or carboxy-terminal peptide bond with said first effector cell binding moiety and said second effector cell binding moiety shares an amino- or carboxy-terminal peptide bond with either a) said protease-activatable IL-2 polypeptide or b) said first effector cell binding moiety; (iii) the protease-activatable IL-2 polypeptide shares an amino- or carboxy-terminal peptide bond with the antigen binding moiety and the effector cell binding moiety shares an amino- or carboxy-terminal peptide bond with either a) said protease-activatable IL-2 polypeptide or b) said antigen binding moiety; or (iv) the protease-activatable IL-2 polypeptide shares an amino- or carboxy-terminal peptide bond with the effector cell binding moiety and the antigen binding moiety shares an amino- or carboxy-terminal peptide bond with either a) said protease-activatable IL-2 polypeptide or b) said effector cell binding moiety.

In one embodiment, the antigen binding moiety or the effector cell binding moiety comprised in the protease-activatable IL-2 polypeptide as disclosed herein or the immunoconjugate as disclosed herein is an antibody or an antibody fragment. In one embodiment, the antigen binding moiety and the effector cell binding moiety comprised in the protease-activatable IL-2 polypeptide as disclosed herein or the immunoconjugate as disclosed herein is an antibody or an antibody fragment. In one embodiment, the antigen binding moiety or the effector cell binding moiety is selected from the group consisting of a Fab molecule and a scFv molecule. In one embodiment, the antigen binding moiety and the effector cell binding moiety is selected from the group consisting of a Fab molecule and a scFv molecule. In one embodiment, the antigen binding moiety or the effector cell binding moiety is an immunoglobulin molecule, particularly an IgG molecule. In one embodiment, the antigen binding moiety and the effector cell binding moiety is an immunoglobulin molecule, particularly an IgG molecule. In one embodiment, the antigen binding moiety is directed to an antigen presented on a tumor cell or in a tumor cell environment or wherein said effector cell binding moiety is directed to an effector cell present in a tumor cell environment in order to achieve cis-targeting. In one embodiment, the antigen binding moiety is directed to an antigen presented on a tumor cell or in a tumor cell environment and wherein said effector cell binding moiety is directed to an effector cell present in a tumor cell environment in order to achieve cis-targeting.

The invention further provides one or more isolated polynucleotides encoding the protease-activatable IL-2 polypeptide as described herein or the immunoconjugate as described herein, one or more expression vectors comprising the polynucleotides described herein, and one or more host cells comprising the polynucleotide as described herein or the expression vector described herein.

Also provided is a method of producing a protease-activatable IL-2 polypeptide or an immunoconjugate as described herein comprising culturing the host cell as described herein under conditions suitable for the expression of the protease-activatable IL-2 polypeptide or the immunoconjugate.

Also provided is a protease-activatable IL-2 polypeptide or immunoconjugate as described herein produced by the method described herein. Also provided is a pharmaceutical composition comprising the protease-activatable IL-2 polypeptide or immunoconjugate as disclosed herein and a pharmaceutically acceptable carrier. In particular, the invention encompasses a protease-activatable IL-2 polypeptide or an immunoconjugate as described herein for use in the treatment of a disease in an individual in need thereof. In a particular embodiment, said disease is cancer. In a particular embodiment, the individual is a human.

Also encompassed by the invention is the use of the protease-activatable IL-2 polypeptide or immunoconjugate as described herein for manufacture of a medicament for treating a disease in an individual in need thereof. Further provided is a method of treating a disease in an individual, comprising administering to said individual a therapeutically effective amount of a composition comprising the protease-activatable IL-2 polypeptide or immunoconjugate as described herein in a pharmaceutically acceptable form. Said disease preferably is cancer.

Also provided is a method of stimulating the immune system of an individual, comprising administering to said individual an effective amount of a composition comprising the protease-activatable IL-2 polypeptide or immunoconjugate as disclosed herein in a pharmaceutically acceptable form.

### BRIEF DESCRIPTION OF THE FIGURES

FIG. 1A-E. FIG. 1A shows a non-masked control construct with C-terminal IL2v (CD8-IL2v OA). This non-masked one-armed CD8-targeted IgG PG LALA with IL2v fused to the C-terminus of the empty Fc knob chain was used as a control for the comparison with masked and matriptase-unmasked constructs. FIG. 1B shows scFv MT204-masked construct with two PMAKK matriptase release sites (CD8-IL2v MT204 2×PMAKK). In this construct, IL2v is fused to the hinge-region of the Fc knob chain and masked by the scFv MT204 linked to the N-terminus of IL2v. One of the two PMAKK matriptase release sites is positioned between IL2v and the VL domain of the scFv MT204, whereas the second is positioned in the linker between the VL and VH domains of the scFv MT204. FIG. 1C shows a scFv MT204-masked construct without matriptase release sites (CD8-IL2v MT204 non cleavable). This construct is similar to CD8-IL2v MT204 2×PMAKK except that it comprises non-cleavable linkers and has been used as a comparator for the non-masked (FIGS. 1A and 1E) and matriptase-unmasked CD8-IL2v MT204 2×PMAKK. FIG. 1D shows a disulfide-stabilized scFv MT204-masked construct with one MMP9/matriptase release site (CD8-IL2v MT204 1×MMP9/Matriptase). This disulfide-stabilized (ds) scFv MT204-masked construct contains only one protease release site for unmasking, more precisely, an MMP9/matriptase release site is positioned between IL2v and the VL domain of the ds-scFv. FIG. 1E shows a non-masked control construct with N-terminal IL2v (IL2v_CD8v11_Fc(kih)). In this construct, IL2v is fused to the hinge-region of the Fc knob chain. It has been used as a comparator to the masked constructs (FIG. 1B-1D).

FIG. 2. Proliferation of the human NK cell line NK92 upon treatment for four days with Matriptase-digested or undigested masked CD8-IL2v constructs determined by luminescence.

FIG. 3A-C. Proliferation of CD4 T cells, CD8 T cells and NK cells within PBMCs upon treatment for five days with Matriptase-digested or non-digested masked CD8-IL2v constructs determined by flow cytometry.

FIG. 4A-C. Activation of CD4 T cells, CD8 T cells and NK cells within PBMCs upon treatment for five days with Matriptase-digested or non-digested masked CD8-IL2v constructs determined by flow cytometry.

FIG. 5. Electronical gels of non-reduced CE-SDS of constructs (FIG. 5A with CD8-IL2v OA;

FIG. 5B with CD8-IL2v MT204 2×PMAKK; FIG. 5C CD8-IL2v MT204 non cleavable) incubated with and without matriptase.

FIG. 6. Human PD1-targeted masked IL2v constructs with PQARK matriptase sites and respective controls. FIG. 6A (P1AG9597) shows an one-armed human PD1-targeted human IgG PG LALA with masked IL2v fused to the N-terminus of the hinge region of the Fc knob chain and two PQARK matriptase sites for the release of the mask; FIG. 6B (PIAG0929) shows an one-armed human PD1-targeted human IgG PG LALA with masked IL2v fused to the N-terminus of the hinge region of the Fc knob chain and without matriptase release sites (non-cleavable control); FIG. 6C (P1AG3071) shows an one-armed human PD1-targeted human IgG PG LALA with IL2v fused to the N-terminus of the hinge region of the Fc knob chain (non-masked control); FIG. 6D (P1AG9606) shows a bivalent human PD1-targeted human IgG PG LALA with masked IL2v (‘in-line’) fused to the C-terminus of the Fc knob chain and two PQARK matriptase sites for the release of the mask; FIG. 6E (P1AG5740) shows a bivalent human PD1-targeted human IgG PG LALA with masked IL2v (‘in-line’) fused to the C-terminus of the Fc knob chain and without matriptase release sites (non-cleavable control); FIG. 6F (P1AG5741 & P1AA7146) shows a bivalent human PD1-targeted human IgG PG LALA with IL2v fused to the C-terminus of the Fc knob chain (non-masked control); FIG. 6G (PIAG9607) shows a bivalent human PD1-targeted human IgG PG LALA with IL2v fused to the C-terminus of the Fc knob chain and the mask fused to the C-terminus of the Fc hole chain (′IL2v and mask on separate chains') and two PQARK matriptase sites for the release of the mask.

FIG. 7. Murine surrogates of human PD1-targeted masked IL2v constructs with PQARK matriptase sites and respective controls. FIG. 7A (PIAG9629) shows an one-armed human PD1-targeted murine IgG DA PG with masked IL2v fused to the N-terminus of the hinge region of the Fc DD-chain and two PQARK matriptase sites for the release of the mask; FIG. 7B (P1AG0905) shows an one-armed human PD1-targeted murine IgG DA PG with masked IL2v fused to the N-terminus of the hinge region of the Fc DD-chain and without matriptase release sites (non-cleavable control); FIG. 7C (PIAG3108) shows an one-armed human PD1-targeted murine IgG DA PG with IL2v fused to the N-terminus of the hinge region of the Fc DD-chain (non-masked control); FIG. 7D (PIAG9983) shows a bivalent human PD1-targeted murine IgG DA PG with masked IL2v (‘in-line’) fused to the C-terminus of the Fc DD-chain and two PQARK matriptase sites for the release of the mask; FIG. 7E (P1AG9984) shows a bivalent human PD1-targeted murine IgG DA PG with masked IL2v (‘in-line’) fused to the C-terminus of the Fc DD-chain and without matriptase release sites (non-cleavable control); FIG. 7F (P1AG7552) shows a bivalent human PD1-targeted murine IgG DA PG with IL2v fused to the C-terminus of the Fc DD-chain (non-masked control).

FIG. 8. Murine surrogates of murine PD1-targeted masked IL2v constructs with PQARK matriptase sites and respective controls. FIG. 8A (PIAG9630) shows an one-armed murine PD1-targeted murine IgG DA PG with masked IL2v fused to the N-terminus of the hinge region of the Fc DD-chain and two PQARK matriptase sites for the release of the mask; FIG. 8B (P1AG0908) shows an one-armed murine PD1-targeted murine IgG DA PG with masked IL2v fused to the N-terminus of the hinge region of the Fc DD-chain and without matriptase release sites (non-cleavable control); FIG. 8C (P1AG3109) shows an one-armed murine PD1-targeted murine IgG DA PG with IL2v fused to the N-terminus of the hinge region of the Fc DD-chain (non-masked control); FIG. 8D (PIAG9989) shows a bivalent murine PD1-targeted murine IgG DA PG with masked IL2v (‘in-line’) fused to the C-terminus of the Fc DD-chain and two PQARK matriptase sites for the release of the mask; FIG. 8E (P1AG9990) shows a bivalent murine PD1-targeted murine IgG DA PG with masked IL2v (‘in-line’) fused to the C-terminus of the Fc DD-chain and without matriptase release sites (non-cleavable control); FIG. 8F (P1AG9991) shows a bivalent murine PD1-targeted murine IgG DA PG with IL2v fused to the C-terminus of the Fc knob chain (non-masked control); FIG. 8G (P1AG9994) shows a bivalent murine PD1-targeted murine IgG DA PG with IL2v fused to the C-terminus of the Fc KK+ chain and the mask fused to the C-terminus of the Fc DD-chain (‘IL2v and mask on separate chains’) and two PQARK matriptase sites for the release of the mask; FIG. 8H (P1AG9995) shows a bivalent murine PD1-targeted murine IgG DA PG with IL2v fused to the C-terminus of the Fc KK+ chain and the mask fused to the C-terminus of the Fc DD-chain (′IL2v and mask on separate chains') and without matriptase release sites (non-cleavable control).

FIG. 9. Human CD8-targeted masked IL2v constructs with PMAKK matriptase sites and respective controls. FIG. 9A (P1AF6882) shows an one-armed human CD8-targeted human IgG PG LALA with masked IL2v fused to the N-terminus of the hinge region of the Fc knob chain and two PMAKK matriptase sites for the release of the mask; FIG. 9B (P1AF6883) shows an one-armed human CD8-targeted human IgG PG LALA with masked IL2v fused to the N-terminus of the hinge region of the Fc knob chain and without matriptase release sites (non-cleavable control); FIG. 9C (P1AF7468) shows an one-armed human CD8-targeted human IgG PG LALA with IL2v fused to the N-terminus of the hinge region of the Fc knob chain (non-masked control).

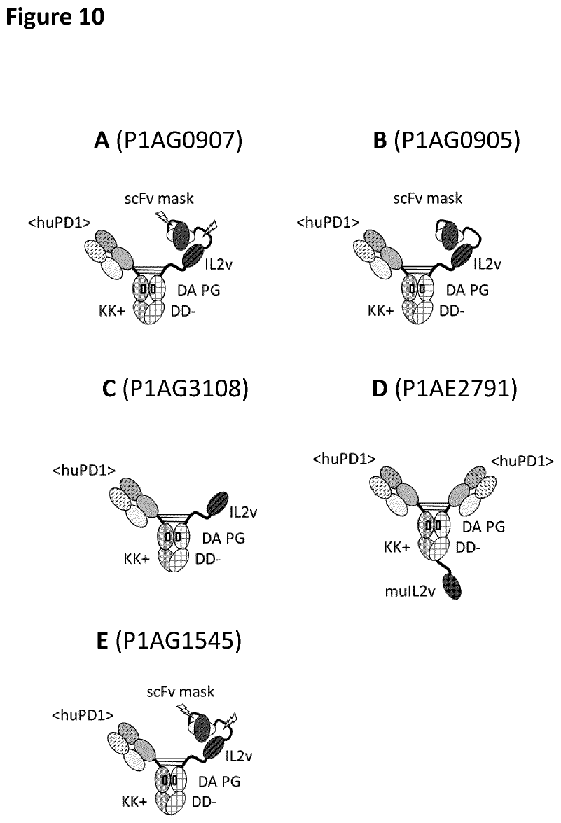

FIG. 10. Murine surrogates of human PD1-targeted masked IL2v constructs with PMAKK or YAARKGGI matriptase sites and respective controls. FIG. 10A (P1AG0907) shows an one-armed human PD1-targeted murine IgG DA PG with masked IL2v fused to the N-terminus of the hinge region of the Fc DD-chain and two PMAKK matriptase sites for the release of the mask; FIG. 10B (PIAG0905) shows an one-armed human PD1-targeted murine IgG DA PG with masked IL2v fused to the N-terminus of the hinge region of the Fc DD-chain and without matriptase release sites (non-cleavable control); FIG. 10C (P1AG3108) shows an one-armed human PD1-targeted murine IgG DA PG with IL2v fused to the N-terminus of the hinge region of the Fc DD-chain (non-masked control); FIG. 10D (P1AE2791) shows a bivalent human PD1-targeted murine IgG DA PG with murine IL2v fused to the C-terminus of the Fc DD-chain (additional non-masked control with murine IL2v); FIG. 10E (P1AG1545) shows an one-armed human PD1-targeted murine IgG DA PG with masked IL2v fused to the N-terminus of the hinge region of the Fc DD-chain and two YAARKGGI matriptase sites for the release of the mask.

FIG. 11. Binding of the indicated constructs to PD1 positive CD4 T cells (FIG. 11A) and CD 8 T cells (FIG. 11B) within PBMCs was determined by flow cytometry. Molecules were detected with a fluorescently labeled anti-human Fc specific secondary antibody.

FIG. 12. Proliferation of the human NK cell line NK92 cell induced by the indicated molecules was measured using CellTiter Glo. FIG. 12A shows undigested material, i.e. matriptase was not added. FIG. 12B shows the matriptase digested material.

FIG. 13. STAT5 phosphorylation in PD1 blocked (FIG. 13A) or PD1 positive (FIG. 13B) CD4 T cells upon treatment with IL2v containing molecules was determined by flow cytometry.

FIG. 14. Binding of the indicated constructs to PD1 positive CD4 (FIG. 14A) and CD 8 T cells (FIG. 14B) within PBMCs was determined by flow cytometry. Molecules were detected with a fluorescently labeled anti-human Fc or anti-mouse specific secondary antibody.

FIG. 15. Proliferation of the human NK cell line NK92 cell induced by the indicated molecules was measured using CellTiter Glo. FIG. 15A and FIG. 15B shows a comparison of matriptase digested and undigested murine TA constructs with PMAKK or YAARKGCCI sites. FIG. 15C and FIG. 15D show murine and human TA constructs with PQARK sites and respective control constructs.

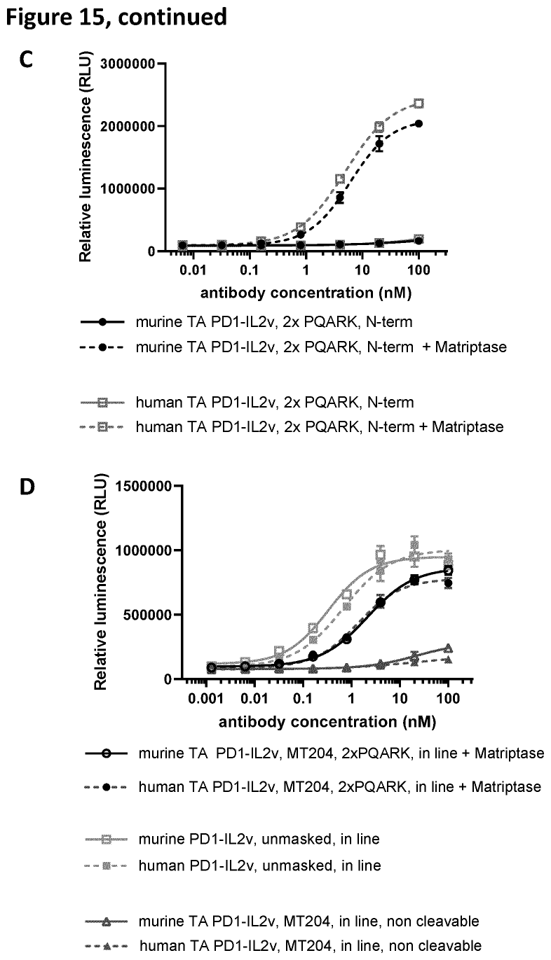

FIG. 16 presents the results of an efficacy experiment with TA-PD1-IL2v cleavable (PMAKK or YAARKGGI linkers), non-cleavable and non-masked Mabs as single agents compared to clinical lead PD1-IL2v. The KPC-4662 pancreatic carcinoma cell line was injected subcutaneously in Black 6-huPD1 tg mice to study tumor growth inhibition in a subcutaneous model. Tumor size was measured using a caliper. Therapy started when tumors reached 200 mm3. The amount of antibodies injected per mouse was 1 mg/kg for TA-PD1-IL2v PMAKK cleavable, TA-PD1-IL2v YAARKGGI cleavable, TA-PD1-IL2v non-masked and PD1-IL2v and 3 mg/kg for TA-PD1-IL2v non-cleavable given once a week. The treatment lasted 3 weeks. The TA-PD-IL2v YARRKGGI mediated superior efficacy in terms of tumor growth inhibition compared to vehicle, non-cleavable and non-masked Mab single agent groups. The TA-PD-IL2v YARRKGGI cleavable linker showed similar tumor growth inhibition as the PD1-IL2v group.

FIG. 17 presents an activity assay of murine interferon-γ constructs characterized by MHC1 (FIG. 17A) and PD-L1 induction (FIG. 17B).

### DETAILED DESCRIPTION

### Definitions

Terms are used herein as generally used in the art, unless otherwise defined in the following.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 13.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO: 13). The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as an IL-2 mutant peptide sequence, an IL-2 mutant polypeptide, IL-2 mutant protein or IL-2 mutant analog.

Designation of various forms of IL-2 is herein made with respect to the sequence shown in SEQ ID NO: 13. Various designations may be used herein to indicate the same mutation. For example a mutation from phenylalanine at position 42 to alanine can be indicated as 42A, A42, A₄₂, F42A, or Phe42Ala.

As used herein, a “wild-type” form of IL-2 is a form of IL-2 that is otherwise the same as the mutant IL-2 polypeptide except that the wild-type form has a wild-type amino acid at each amino acid position of the mutant IL-2 polypeptide. For example, if the IL-2 mutant is the full-length IL-2 (i.e. IL-2 not fused or conjugated to any other molecule), the wild-type form of this mutant is full-length native IL-2. If the IL-2 mutant is a fusion between IL-2 and another polypeptide encoded downstream of IL-2 (e.g. an antibody chain) the wild-type form of this IL-2 mutant is IL-2 with a wild-type amino acid sequence fused to the same downstream polypeptide. Furthermore, if the IL-2 mutant is a truncated form of IL-2 (the mutated or modified sequence within the non-truncated portion of IL-2) then the wild-type form of this IL-2 mutant is a similarly truncated IL-2 that has a wild-type sequence. For the purpose of comparing IL-2 receptor binding affinity or biological activity of various forms of IL-2 mutants to the corresponding wild-type form of IL-2, the term wild-type encompasses forms of IL-2 comprising one or more amino acid mutation that does not affect IL-2 receptor binding compared to the naturally occurring, native IL-2, such as e.g. a substitution of cysteine at a position corresponding to residue 125 of human IL-2 to alanine. In some embodiments wild-type IL-2 for the purpose of the present invention comprises the amino acid substitution C125A. In certain embodiments according to the invention the wild-type IL-2 polypeptide to which the mutant IL-2 polypeptide is compared comprises the amino acid sequence of SEQ ID NO: 13.

The term “CD25” or “α-subunit of the IL-2 receptor” as used herein, refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments CD25 is human CD25.

The term “high-affinity IL-2 receptor” as used herein refers to the heterotrimeric form of the IL-2 receptor, consisting of the receptor γ-subunit (also known as common cytokine receptor γ-subunit, γ_{c}, or CD132), the receptor β-subunit (also known as CD122 or p70) and the receptor α-subunit (also known as CD25 or p55). The term “intermediate-affinity IL-2 receptor” by contrast refers to the IL-2 receptor including only the γ-subunit and the β-subunit, without the α-subunit (for a review see e.g. Olejniczak and Kasprzak, Med Sci Monit 14, RA179-189 (2008)).

By “regulatory T cell” or “T_{reg} cell” is meant a specialized type of CD4⁺ T cell that can suppress the responses of other T cells. T_{reg} cells are characterized by expression of the α-subunit of the IL-2 receptor (CD25) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. T_{reg} cells require IL-2 for their function and development and induction of their suppressive characteristics.

As used herein, the term “effector cells” refers to a population of lymphocytes that mediate the cytotoxic effects of IL-2. Effector cells include effector T cells such as CD8 cytotoxic T cells, NK cells, lymphokine-activated killer (LAK) cells and macrophages/monocytes.

As used herein, the term “antigen binding molecule” refers in its broadest sense to a molecule that specifically binds an antigenic determinant. Examples of antigen binding molecules are immunoglobulins and derivatives, e.g., fragments, thereof.

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigen binding moiety” refers to a polypeptide molecule that specifically binds to an antigenic determinant. In one embodiment, an antigen binding moiety is able to direct the entity to which it is attached (e.g., a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ. As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be any native form of the proteins from any vertebrate source, including mammals such as primates (e.g., humans) and rodents (e.g., mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g., splice variants or allelic variants. The ability of an antigen binding moiety to bind to a specific antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers.

A “target cell antigen” as used herein refers to an antigenic determinant presented on the surface of a target cell, for example a cell in a tumor such as a cancer cell or a cell of the tumor stroma.

As used herein, the terms “first” and “second” with respect to antigen binding moieties etc., are used for convenience of distinguishing when there is more than one of each type of moiety. Use of these terms is not intended to confer a specific order or orientation of the protease-activatable IL-2 polypeptides or immunoconjugates unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

“TA” stands for tumor-activatable.

By “fused” is meant that the components (e.g., a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. Another term is single-chain variable fragments (scFv). In a particular such embodiment, the C-terminus of the Fab light chain is connected to the N-terminus of the Fab heavy chain in the single-chain Fab molecule.

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g., γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antibody” herein is used in the broadest sense and encompasses various antibody structures, including but not limited to monoclonal antibodies, polyclonal antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and single-domain antibodies. For a review of certain antibody fragments, see Hudson et al., Nat Med 9, 129-134 (2003). For a review of scFv fragments, see e.g., Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g., U.S. Pat. No. 6,248,516 B1). Antibody fragments can be made by various techniques, including but not limited to proteolytic digestion of an intact antibody as well as production by recombinant host cells (e.g., E. coli or phage), as described herein.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

The polypeptide sequences of the sequence listing are not numbered according to the Kabat numbering system. However, it is well within the ordinary skill of one in the art to convert the numbering of the sequences of the Sequence Listing to Kabat numbering.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

By “fused” is meant that the components (e.g. a Fab molecule and an Fc domain subunit) are linked by peptide bonds, either directly or via one or more peptide linkers.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

As used herein, the term “immunoconjugate” refers to a polypeptide molecule that includes at least one IL-2 moiety and at least one antigen binding moiety or effector cell binding moiety. In certain embodiments, the immunoconjugate comprises at least one IL-2 moiety, and at least two antigen binding moieties or at least two effector cell binding moieties. Particular immunoconjugates according to the invention essentially consist of one IL-2 moiety and two antigen binding moieties joined by one or more linker sequences. The antigen binding moiety can be joined to the IL-2 moiety by a variety of interactions and in a variety of configurations as described herein. Particular immunoconjugates according to the invention essentially consist of one IL-2 moiety and two effector cell binding moieties joined by one or more linker sequences. The effector cell binding moiety can be joined to the IL-2 moiety by a variety of interactions and in a variety of configurations as described herein.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g., the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term “nucleic acid molecule” refers to any one or more nucleic acid segments, e.g., DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g., ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g., PCT publication no. WO 2006/082515 or PCT publication no. WO 2012/130831).

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, protease-activatable IL-2 polypeptides or immunoconjugates of the invention are used to delay development of a disease or to slow the progression of a disease.

The term “package insert” is used to refer to instructions customarily included in commercial packages of therapeutic products, that contain information about the indications, usage, dosage, administration, combination therapy, contraindications and/or warnings concerning the use of such therapeutic products.

An “idiotype-specific polypeptide” as used herein refers to a polypeptide that recognizes the idiotype of an antigen-binding moiety, e.g., an antigen-binding moiety specific for CD3. The idiotype-specific polypeptide is capable of specifically binding to the variable region of the antigen-binding moiety and thereby reducing or preventing specific binding of the antigen-binding moiety to its cognate antigen. When associated with a molecule that comprises the antigen-binding moiety, the idiotype-specific polypeptide can function as a masking moiety of the molecule. Specifically disclosed herein are anti-idiotype antibodies or anti-idiotype-binding antibody fragments specific for the idiotype of anti-CD3 binding molecules.

“Protease” or “proteolytic enzyme” as used herein refers to any proteolytic enzyme that cleaves the linker at a recognition site and that is expressed by a target cell. Such proteases might be secreted by the target cell or remain associated with the target cell, e.g., on the target cell surface. Examples of proteases include but are not limited to metalloproteinases, e.g., matrix metalloproteinase 1-28 and A Disintegrin And Metalloproteinase (ADAM) 2, 7-12, 15, 17-23, 28-30 and 33, serine proteases, e.g., urokinase-type plasminogen activator and Matriptase, cysteine protease, aspartic proteases, and members of the cathepsin family.

“Protease activatable” as used herein, with respect to the interleukin-2 polypeptides, refers to an interleukin-2 polypeptides having reduced or abrogated ability to bind the interleukin-2 receptor due to a masking moiety that reduces or abrogates the interleukin-2 polypeptides's ability to bind to the interleukin-2 receptor. Upon dissociation of the masking moiety by proteolytic cleavage, e.g., by proteolytic cleavage of a linker connecting the masking moiety to the interleukin-2 polypeptide and or within the masking moiety, binding to the interleukin-2 receptor is restored and the interleukin-2 polypeptide is thereby activated.

“Reversibly concealing” as used herein refers to the binding of a masking moiety to an interleukin-2 polypeptide such as to prevent the interleukin-2 polypeptide from binding to its receptor. This concealing is reversible in that the masking moiety can be released from the interleukin-2 polypeptide, e.g. by protease cleavage, and thereby freeing the interleukin-2 polypeptide to bind to its receptor.

### EMBODIMENTS OF THE DISCLOSURE

In one aspect, provided is an isolated polypeptide comprising a protease recognition site. In one embodiment, the protease recognition site is a substrate for matriptase. In one embodiment the protease recognition site comprises or consists of the sequence PQARK (SEQ ID NO: 32) or HQARK (SEQ ID NO: 33). In one embodiment, the isolated polypeptide comprises one or several unstructured peptide linkers. In one embodiment, the isolated polypeptide comprises at least one linker, in particular wherein the at least one linker does not exhibit secondary structure.

In one embodiment, the linker is a peptide with an amino acid sequence with a length of at least 5 amino acids, preferably with a length of 5 to 100, more preferably of 10 to 50 amino acids, most preferably of 20 to 40. In one embodiment, the protease cleavable linker is a peptide with a length of 30, 31, 32, 33, 34, 35, 36, 37, 38, 39 or 40 amino acids. In a preferred embodiment, the protease cleavable linker is a peptide with a length of 33 amino acids. In one embodiment, the isolated polypeptide comprises a protease cleavable linker.

In one embodiment the protease cleavable linker comprises a protease recognition site. In one embodiment, the protease recognition sequence is a substrate for matriptase. In one embodiment the protease recognition site comprises or consists of the sequence PQARK (SEQ ID NO: 32) or HQARK (SEQ ID NO: 33).

In one embodiment, the protease cleavable linker is an unstructured polypeptide. In one embodiment, the protease cleavable linker does not exhibit secondary structure. In one embodiment the protease cleavable linker comprises at least one linker that promote an unstructured confirmation. In one embodiment, the linker comprises serine(S) and/or glycine (G). In one embodiment, the protease cleavable linker at least one linker comprising an amino acid sequence (GxS)n or (GxS)nGm with G=glycine, S=serine, and (x=3, n=3, 4, 5 or 6, and m=0, 1, 2 or 3) or (x=4, n=2, 3, 4 or 5 and m=0, 1, 2 or 3), preferably x=4 and n=2 or 3, more preferably with x=4, n=2. In one embodiment the protease cleavable linker comprises (G4S)2. In one embodiment the protease cleavable linker comprises (G4S)3. In one embodiment the protease cleavable linker comprises G2S. The protease-cleavable linker comprises the protease recognition site at any position (e.g. at the start, within at any position, or at the end of the linker).

In one embodiment, isolated polypeptide comprises or consists of the sequence GGGGSGGGGSGGGPQARKGGGGGGSGGGGG (SEQ ID NO: 102). In one embodiment, isolated polypeptide comprises of or consists the sequence GGGGSGGGGSPQARKGGGGSGGGGSGGGGSGGS (SEQ ID NO: 110). In one embodiment, the isolated polypeptide comprises or consists of the sequence GGGGSGGGGSHQARKGGGGSGGGGSGGGGSGGS (SEQ ID NO: 111)

In one aspect, the invention relates to a protease-activatable interleukin-2 (IL-2) polypeptide comprising (i) an IL-2 polypeptide, (ii) a masking moiety and (iii) a linker comprising a first protease cleavage site, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site.

In one embodiment, In one aspect, the invention relates to a protease-activatable interleukin-2 (IL-2) polypeptide comprising (i) an IL-2 polypeptide, (ii) a masking moiety and (iii) a linker comprising a first protease cleavage site, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and the second protease cleavage site.

In one embodiment, In one aspect, the invention relates to a protease-activatable interleukin-2 (IL-2) polypeptide comprising (i) an IL-2 polypeptide, (ii) a masking moiety and (iii) a linker comprising a first protease cleavage site, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first or the second protease cleavage site.

In a preferred embodiment, the protease-activatable interleukin-2 polypeptide comprises the amino acid sequence of SEQ ID NO: 9.

### Immunoconjugates

In one aspect, the invention relates to an immunoconjugate comprising a protease activatable IL-2 polypeptide and an antigen binding moiety and/or an effector cell binding moiety.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 4, a polypeptide comprising an amino acid sequence that is at least about 80%, 85%, 90%, 95%, 96%, 97%, 98%, 99% or 100% identical to the sequence of SEQ ID NO: 2, and a polypeptide comprising an amino acid sequence that is at least about 80%, 85%, 90%, 95%, 96%, 97%, 98%, 99% or 100% identical to the sequence of SEQ ID NO: 5.

In a specific embodiment, the invention provides an immunoconjugate comprising a polypeptide comprising an amino acid sequence of SEQ ID NO: 4, a polypeptide comprising an amino acid sequence of SEQ ID NO: 2, and a polypeptide comprising an amino acid sequence of SEQ ID NO: 5.

### Masking Moiety

The protease-activatable IL-2 polypeptide of the invention comprises at least one masking moiety.

In one embodiment, the masking moiety masks the IL-2 polypeptide and comprises at least one of the heavy chain CDR1, the heavy chain CDR2, the heavy chain CDR3, the light chain CDR1, the light chain CDR2, and the light chain CDR3 of the MT204 antibody. In one embodiment, the masking moiety comprises the heavy chain CDR1, the heavy chain CDR2, the heavy chain CDR3, the light chain CDR1, the light chain CDR2, and the light chain CDR3 of the MT204 antibody.

In one embodiment, the masking moiety masks the IL-2 polypeptide and comprises at least one of the heavy chain variable region and the light chain variable region of the MT204 antibody. In one embodiment, the masking moiety masks the IL-2 polypeptide and comprises the heavy chain variable region and the light chain variable region of the MT204 antibody. In one embodiment, the masking moiety masks the IL-2 polypeptide and comprises the heavy chain variable region and the light chain variable region of the MT204 antibody, wherein the MT204 antibody is a single-chain Fab molecule.

In one embodiment, the masking moiety masks the IL-2 polypeptide and comprises a polypeptide sequence that is at least about 95%, 96%, 97%, 98%, 99% or 100% identical to SEQ ID NO: 12. In one embodiment, the masking moiety masks the IL-2 polypeptide and comprises the polypeptide sequence of SEQ ID NO: 12.

### Linkers

In one embodiment, the protease-activatable IL-2 polypeptide or the immunoconjugate comprises a linker having a protease recognition site comprising a polypeptide sequence that is at least about 95%, 96%, 97%, 98%, 99% or 100% identical to SEQ ID NO: 15, 16, 17, 18, 19, 20, 21, 22, 23, 24, 25, 26, 27, 28, 29, 30, 31, 32 or 33. In one embodiment, the protease recognition site comprises the polypeptide sequence of SEQ ID NO: 15, 16, 17, 18, 19, 20, 21, 22, 23, 24, 25, 26, 27, 28, 29, 30, 31, 32 or 33. In a preferred embodiment, the protease recognition site comprises the polypeptide sequence of SEQ ID NO: 30.

In one embodiment the protease is selected from the group consisting of metalloproteinase, e.g., matrix metalloproteinase (MMP) 1-28 and A Disintegrin And Metalloproteinase (ADAM) 2, 7-12, 15, 17-23, 28-30 and 33, serine protease, e.g., urokinase-type plasminogen activator and Matriptase, cysteine protease, aspartic protease, and cathepsin protease. In one specific embodiment the protease is MMP9 or MMP2. In a further specific embodiment, the protease is Matriptase.

### Polynucleotides

The invention further provides isolated polynucleotides encoding a protease-activatable IL-2 polypeptide or immunoconjugate as described herein or a fragment thereof.

The polynucleotides encoding protease-activatable IL-2 polypeptides or immunoconjugates of the invention may be expressed as a single polynucleotide that encodes the entire protease-activatable IL-2 polypeptides or immunoconjugates or as multiple (e.g., two or more) polynucleotides that are co-expressed. Polypeptides encoded by polynucleotides that are co-expressed may associate through, e.g., disulfide bonds or other means to form a functional protease-activatable IL-2 polypeptides or immunoconjugates. In the context of an immunoconjugate for example, the light chain portion of an antigen binding moiety may be encoded by a separate polynucleotide from the polynucleotide encoding the heavy chain of the immunoconjugate, an Fc domain subunit and optionally (part of) another antigen binding moiety. When co-expressed, the heavy chain polypeptides will associate with the light chain polypeptides to form the antigen binding moiety. In another example, the portion immunoconjugate comprising one of the two Fc domain subunits and optionally (part of) one or more antigen binding moieties could be encoded by a separate polynucleotide from the portion of the immunoconjugate comprising the other of the two Fc domain subunits and optionally (part of) an antigen binding moiety. When co-expressed, the Fc domain subunits will associate to form the Fc domain.

In some embodiments, the isolated polynucleotide encodes the entire immunoconjugate according to the invention as described herein. In other embodiments, the isolated polynucleotide encodes a polypeptides comprised in the immunoconjugate according to the invention as described herein.

In another embodiment, the present invention is directed to an isolated polynucleotide encoding a protease-activatable IL-2 polypeptide or immunoconjugate of the invention or a fragment thereof. In another embodiment, the present invention is directed to an isolated polynucleotide encoding a sequence that encodes a polypeptide sequence as shown in SEQ ID NO: 9 or a fragment thereof. In another embodiment, the present invention is directed to an isolated polynucleotide encoding a sequence that encodes a polypeptide sequence as shown in SEQ ID NO: 12 or a fragment thereof.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

The IL-2 polypeptide or immunoconjugate of the invention may be obtained, for example, by solid-state peptide synthesis (e.g., Merrifield solid phase synthesis) or recombinant production. For recombinant production one or more polynucleotide encoding the protease-activatable IL-2 polypeptide or immunoconjugate, e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a IL-2 polypeptide or immunoconjugate along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the protease-activatable IL-2 polypeptide or immunoconjugate (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the protease-activatable IL-2 polypeptide or immunoconjugate of the invention, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the IL-2 polypeptide or immunoconjugate is desired, DNA encoding a signal sequence may be placed upstream of the nucleic acid encoding a protease-activatable IL-2 polypeptide or immunoconjugate of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g., a histidine tag) or assist in labeling the protease-activatable IL-2 polypeptide or immunoconjugate may be included within or at the ends of the protease-activatable IL-2 polypeptide or immunoconjugate encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g., has been transformed or transfected with) a vector comprising a polynucleotide that encodes (part of) a protease-activatable IL-2 polypeptide or immunoconjugate of the invention. As used herein, the term “host cell” refers to any kind of cellular system which can be engineered to generate the protease-activatable IL-2 polypeptide or immunoconjugate of the invention or fragments thereof. Host cells suitable for replicating and for supporting expression of protease-activatable IL-2 polypeptides or immunoconjugates are well known in the art. Such cells may be transfected or transduced as appropriate with the particular expression vector and large quantities of vector containing cells can be grown for seeding large scale fermenters to obtain sufficient quantities of the IL-2 polypeptide or immunoconjugate for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NS0, Sp20 cell).

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a protease-IL-2 polypeptide or immunoconjugate according to the invention is provided, wherein the method comprises culturing a host cell comprising a polynucleotide encoding the protease-activatable IL-2 polypeptide or immunoconjugate, as provided herein, under conditions suitable for expression of the protease-activatable IL-2 polypeptide or immunoconjugate, and recovering the protease-activatable IL-2 polypeptide or immunoconjugate from the host cell (or host cell culture medium).

The components of the protease-activatable IL-2 polypeptide or immunoconjugate are genetically fused to each other. Protease-activatable IL-2 polypeptides or immunoconjugates can be designed such that its components are fused directly to each other or indirectly through a linker sequence. The composition and length of the linker may be determined in accordance with methods well known in the art and may be tested for efficacy. Examples of linker sequences between different components of protease-activatable IL-2 polypeptides or immunoconjugates are found in the sequences provided herein. Additional sequences may also be included to incorporate a cleavage site to separate the individual components of the fusion if desired, for example an endopeptidase recognition sequence.

In certain embodiments the one or more antigen binding moieties of the immunoconjugates comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g., Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g., as described in U.S. Pat. No. 4,186,567) or can be obtained, for example, by screening combinatorial libraries comprising variable heavy chains and variable light chains (see e.g., U.S. Pat. No. 5,969,108 to McCafferty).

Any animal species of antibody, antibody fragment, antigen binding domain or variable region can be used in the immunoconjugates of the invention. Non-limiting antibodies, antibody fragments, antigen binding domains or variable regions useful in the present invention can be of murine, primate, or human origin. If the protease-activatable IL-2 polypeptide or immunoconjugate is intended for human use, a chimeric form of antibody may be used wherein the constant regions of the antibody are from a human. A “humanized” or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g., recipient antibody) framework and constant regions with or without retention of critical framework residues (e.g., those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31 (3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g., Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g., Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, NJ, 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the immunoconjugate of the invention to bind to a specific antigenic determinant can be measured either through an enzyme-linked immunosorbent assay (ELISA) or other techniques familiar to one of skill in the art, e.g., surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen. In certain embodiments, such a competing antibody binds to the same epitope (e.g., a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ). Protease-activatable IL-2 polypeptides or immunoconjugate s prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the protease-activatable IL-2 polypeptide or immunoconjugate binds. For example, for affinity chromatography purification of protease-activatable IL-2 polypeptides or immunoconjugates of the invention, a matrix with protein A or protein G may be used. Sequential Protein A or G affinity chromatography and size exclusion chromatography can be used to isolate a protease-activatable IL-2 polypeptide or immunoconjugate. The purity of the protease-activatable IL-2 polypeptide or immunoconjugate can be determined by any of a variety of well-known analytical methods including gel electrophoresis, high pressure liquid chromatography, and the like.

### Assays

Protease-activatable IL-2 polypeptides or immunoconjugates provided herein may be identified, screened for, or characterized for their physical/chemical properties and/or biological activities by various assays known in the art.

### Affinity Assays

The affinity of the immunoconjugate for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of protease-activatable IL-2 polypeptides or immunoconjugates for different receptors or target antigens may be evaluated using cell lines expressing the particular receptor or target antigen, for example by flow cytometry (FACS). A specific illustrative and exemplary embodiment for measuring binding affinity is described in the following.

According to one embodiment, K_{D} is measured by surface plasmon resonance using a BIACORE® T100 machine (GE Healthcare) at 25° C.

To analyze the interaction between the Fc-portion and Fc receptors, His-tagged recombinant Fc-receptor is captured by an anti-Penta His antibody (Qiagen) immobilized on CM5 chips and the bispecific constructs are used as analytes. Briefly, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier's instructions. Anti Penta-His antibody is diluted with 10 mM sodium acetate, pH 5.0, to 40 μg/ml before injection at a flow rate of 5 μl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05% Surfactant P20, pH 7.4) at 25° C. at a flow rate of 30 μl/min for 120 s.

To determine the affinity to the target antigen, bispecific constructs are captured by an anti-human Fab specific antibody (GE Healthcare) that is immobilized on an activated CM5-sensor chip surface as described for the anti Penta-His antibody. The final amount of coupled protein is is approximately 12000 RU. The bispecific constructs are captured for 90 s at 300 nM. The target antigens are passed through the flow cells for 180 s at a concentration range from 250 to 1000 nM with a flowrate of 30 μl/min. The dissociation is monitored for 180 s.

Bulk refractive index differences are corrected for by subtracting the response obtained on reference flow cell. The steady state response was used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

### Activity Assays

Biological activity of the protease-activatable IL-2 polypeptides or immunoconjugates of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

### Compositions, Formulations, and Routes of Administration

In a further aspect, the invention provides pharmaceutical compositions comprising any of the protease-activatable IL-2 polypeptides or immunoconjugates provided herein, e.g., for use in any of the below therapeutic methods. In one embodiment, a pharmaceutical composition comprises any of the protease-activatable IL-2 polypeptides or immunoconjugates provided herein and a pharmaceutically acceptable carrier. In another embodiment, a pharmaceutical composition comprises any of the protease-activatable IL-2 polypeptides or immunoconjugates provided herein and at least one additional therapeutic agent, e.g., as described below.

Further provided is a method of producing a protease-activatable IL-2 polypeptide or immunoconjugate of the invention in a form suitable for administration in vivo, the method comprising (a) obtaining a protease-activatable IL-2 polypeptide or immunoconjugate according to the invention, and (b) formulating the protease-activatable IL-2 polypeptide or immunoconjugate with at least one pharmaceutically acceptable carrier, whereby a preparation of protease-activatable IL-2 polypeptide or immunoconjugate is formulated for administration in vivo.

Pharmaceutical compositions of the present invention comprise a therapeutically effective amount of one or more protease-activatable IL-2 polypeptide or immunoconjugate dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one protease-activatable IL-2 polypeptide or immunoconjugate and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g., antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. Protease-activatable IL-2 polypeptides or immunoconjugates of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the protease-activatable IL-2 polypeptide or immunoconjugate of the invention.

Parenteral compositions include those designed for administration by injection, e.g., subcutaneous, intradermal, intralesional, intravenous, intraarterial intramuscular, intrathecal or intraperitoneal injection. For injection, the protease-activatable IL-2 polypeptides or immunoconjugates of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the protease-activatable IL-2 polypeptides or immunoconjugates may be in powder form for constitution with a suitable vehicle, e.g., sterile pyrogen-free water, before use. Sterile injectable solutions are prepared by incorporating the protease-activatable IL-2 polypeptides or immunoconjugates of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g., films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the protease-activatable IL-2 polypeptides or immunoconjugates may also be formulated as a depot preparation. Such long acting formulations may be administered by implantation (for example subcutaneously or intramuscularly) or by intramuscular injection. Thus, for example, the protease-activatable IL-2 polypeptides or immunoconjugates may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the protease-activatable IL-2 polypeptides or immunoconjugates of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The protease-activatable IL-2 polypeptides or immunoconjugates may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the protease-activatable IL-2 polypeptides or immunoconjugates provided herein may be used in therapeutic methods. Protease-activatable IL-2 polypeptides or immunoconjugates of the invention can be used as immunotherapeutic agents, for example in the treatment of cancers.

For use in therapeutic methods, protease-activatable IL-2 polypeptides or immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, protease-activatable IL-2 polypeptides or immunoconjugates of the invention for use as a medicament are provided. In further aspects, protease-activatable IL-2 polypeptides or immunoconjugates of the invention for use in treating a disease are provided. In certain embodiments, protease-activatable IL-2 polypeptides or immunoconjugates of the invention for use in a method of treatment are provided. In one embodiment, the invention provides a protease-activatable IL-2 polypeptide or immunoconjugate as described herein for use in the treatment of a disease in an individual in need thereof. In certain embodiments, the invention provides a protease-activatable IL-2 polypeptide or immunoconjugate for use in a method of treating an individual having a disease comprising administering to the individual a therapeutically effective amount of the protease-activatable IL-2 polypeptide or immunoconjugate. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a protease-activatable IL-2 polypeptide or immunoconjugate as described herein for use in inducing lysis of a target cell, particularly a tumor cell. In certain embodiments, the invention provides a protease-activatable IL-2 polypeptide or immunoconjugate for use in a method of inducing lysis of a target cell, particularly a tumor cell, in an individual comprising administering to the individual an effective amount of the protease-activatable IL-2 polypeptide or immunoconjugate to induce lysis of a target cell. An “individual” according to any of the above embodiments is a mammal, preferably a human.

In a further aspect, the invention provides for the use of a protease-activatable IL-2 polypeptide or immunoconjugate of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a tumor cell. In still a further embodiment, the medicament is for use in a method of inducing lysis of a target cell, particularly a tumor cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of a protease-activatable IL-2 polypeptide or immunoconjugate of the invention. In one embodiment a composition is administered to said individual, comprising the protease-activatable IL-2 polypeptide or immunoconjugate of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human. In a further aspect, the invention provides a method for inducing lysis of a target cell, particularly a tumor cell.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a protease-activatable IL-2 polypeptide or immunoconjugate of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the protease-activatable IL-2 polypeptide or immunoconjugate may not provide a cure but may only provide partial benefit. In some embodiments, a physiological change having some benefit is also considered therapeutically beneficial. Thus, in some embodiments, an amount of protease-activatable IL-2 polypeptide or immunoconjugate that provides a physiological change is considered an “effective amount” or a “therapeutically effective amount”. The subject, patient, or individual in need of treatment is typically a mammal, more specifically a human.

In some embodiments, an effective amount of a protease-activatable IL-2 polypeptide or immunoconjugate of the invention is administered to a cell. In other embodiments, a therapeutically effective amount of a protease-activatable IL-2 polypeptide or immunoconjugate of the invention is administered to an individual for the treatment of disease.

For the prevention or treatment of disease, the appropriate dosage of a protease-activatable IL-2 polypeptide or immunoconjugate of the invention (when used alone or in combination with one or more other additional therapeutic agents) will depend on the type of disease to be treated, the route of administration, the body weight of the patient, the type of IL-2 polypeptide or immunoconjugate, the severity and course of the disease, whether the IL-2 polypeptide or immunoconjugate is administered for preventive or therapeutic purposes, previous or concurrent therapeutic interventions, the patient's clinical history and response to the protease-activatable IL-2 polypeptide or immunoconjugate and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

A therapeutically effective dose of the protease-activatable IL-2 polypeptides or immunoconjugates described herein will generally provide therapeutic benefit without causing substantial toxicity. Toxicity and therapeutic efficacy of a protease-activatable IL-2 polypeptide or immunoconjugate can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Protease-activatable IL-2 polypeptides or immunoconjugates that exhibit large therapeutic indices are preferred. In one embodiment, the protease-activatable IL-2 polypeptide or immunoconjugate according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with protease-activatable IL-2 polypeptides or immunoconjugates of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The protease-activatable IL-2 polypeptides or immunoconjugates of the invention may be administered in combination with one or more other agents in therapy. For instance, a protease-activatable IL-2 polypeptide or immunoconjugate of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of protease-activatable IL-2 polypeptide or immunoconjugate used, the type of disorder or treatment, and other factors discussed above. The protease-activatable IL-2 polypeptide or immunoconjugate are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the protease-activatable IL-2 polypeptide or immunoconjugate of the invention can occur prior to, simultaneously, and/or following, administration of the additional therapeutic agent and/or adjuvant. Protease-activatable IL-2 polypeptides or immunoconjugates of the invention can also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a protease-activatable IL-2 polypeptide or immunoconjugate of the invention. The label or package insert indicates that the composition is used for treating the condition of choice. Moreover, the article of manufacture may comprise (a) a first container with a composition contained therein, wherein the composition comprises a protease-activatable IL-2 polypeptide or immunoconjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Sequences

| Name | Sequence | SEQ ID NO |
|---|---|---|
| CD8-IL2v OA | DVQITQSPSSLSASVGDRVTITCRTSRSISQYLAWYQEKP | 1 |
| (LC) I | GKTNKLLIYSGSTLQSGIPSRFSGSGSGTDFTLTISSLQPE | |
| | DFATYYCQQHNENPLTFGQGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACE | |
| | VTHQGLSSPVTKSFNRGEC | |
| CD8-IL2v OA | EVQLVQSGAEVKKPGASVKVSCKASGFNIKDTYIHWVR | 2 |
| (HC hole) | QAPGQGLEWIGRIDPANDNTLYASKFQGRATITADTSTS | |
| | TAYLELSSLRSEDTAVYYCGRGYGYYVFDHWGQGTLV | |
| | TVSSASTKGPSVFPLAPSSKSTSGGTAALGCLVKDYFPE | |
| | PVTVSWNSGALTSGVHTFPAVLQSSGLYSLSSVVTVPSS | |
| | SLGTQTYICNVNHKPSNTKVDKKVEPKSCDKTHTCPPC | |
| | PAPEAAGGPSVFLFPPKPKDTLMISRTPEVTCVVVDVSH | |
| | EDPEVKFNWYVDGVEVHNAKTKPREEQYNSTYRVVSV | |
| | LTVLHQDWLNGKEYKCKVSNKALGAPIEKTISKAKGQP | |
| | REPQVCTLPPSRDELTKNQVSLSCAVKGFYPSDIAVEWE | |
| | SNGQPENNYKTTPPVLDSDGSFFLVSKLTVDKSRWQQG | |
| | NVFSCSVMHEALHNHYTQKSLSLSPGK | |
| CD8-IL2v OA | DKTHTCPPCPAPEAAGGPSVFLFPPKPKDTLMISRTPEVT | 3 |
| (Fc knob- | CVVVDVSHEDPEVKFNWYVDGVEVHNAKTKPREEQY | |
| IL2v) | NSTYRVVSVLTVLHQDWLNGKEYKCKVSNKALGAPIE | |
| | KTISKAKGQPREPQVYTLPPCRDELTKNQVSLWCLVKG | |
| | FYPSDIAVEWESNGQPENNYKTTPPVLDSDGSFFLYSKL | |
| | TVDKSRWQQGNVFSCSVMHEALHNHYTQKSLSLSPGG | |
| | GGGSGGGGSGGGGSAPASSSTKKTQLQLEHLLLDLQMI | |
| | LNGINNYKNPKLTRMLTAKFAMPKKATELKHLQCLEEE | |
| | LKPLEEVLNGAQSKNFHLRPRDLISNINVIVLELKGSETT | |
| | FMCEYADETATIVEFLNRWITFAQSIISTLT | |
| CD8-IL2v (LC) | DVQITQSPSSLSASVGDRVTITCRTSRSISQYLAWYQEKP | 4 |
| II | GKTNKLLIYSGSTLQSGIPSRFSGSGSGTDFTLTISSLQPE | |
| | DFATYYCQQVNEFPPTFGQGTKVEIKRTVAAPSVFIFPPS | |
| | DEQLKSGTASVVCLLNNFYPREAKVQWKVDNALQSGN | |
| | SQESVTEQDSKDSTYSLSSTLTLSKADYEKHKVYACEV | |
| | THQGLSSPVTKSFNRGEC | |
| CD8-IL2v | EVQLVESGGGLVQPGGSLRLSCAASGFTFSSYTLAWVR | 5 |
| MT204 | QAPGKGLEWVAAIDSSSYTYSPDTVRGRFTISRDNAKN | |
| 2xPMAKK | SLYLQMNSLRAEDTAVYYCARDSNWDALDYWGQGTT | |
| (scFv mask- | VTVSSGGGGSGGPMAKKGGGGSGGGDIQMTQSPSSLS | |
| IL2v-Fc knob) | ASVGDRVTITCKASQNVGTNVGWYQQKPGKAPKALIY | |
| | SASFRYSGVPSRFSGSGSGTDFTLTISSLQPEDFATYYCQ | |
| | QYYTYPYTFGGGTKVEIKGGGGSGGGGSPMAKKGGGG | |
| | SGGGGSGGGGSGGGGSGGGAPASSSTKKTQLQLEHLLL | |
| | DLQMILNGINNYKNPKLTRMLTAKFAMPKKATELKHL | |
| | QCLEEELKPLEEVLNGAQSKNFHLRPRDLISNINVIVLEL | |
| | KGSETTFMCEYADETATIVEFLNRWITFAQSIISTLTGGG | |
| | GSGGGGSDKTHTCPPCPAPEAAGGPSVFLFPP | |
| | KPKDTLMISRTPEVTCVVVDVSHEDPEVKFNWYVDGV | |
| | EVHNAKTKPREEQYNSTYRVVSVLTVLHQDWLNGKEY | |
| | KCKVSNKALGAPIEKTISKAKGQPREPQVYTLPPCRDEL | |
| | TKNQVSLWCLVKGFYPSDIAVEWESNGQPENNYKTTPP | |
| | VLDSDGSFFLYSKLTVDKSRWQQGNVFSCSVMHEALH | |
| | NHYTQKSLSLSPGK | |
| CD8-IL2v | EVQLVESGGGLVQPGGSLRLSCAASGFTFSSYTLAWVR | 6 |
| MT204 non | QAPGKGLEWVAAIDSSSYTYSPDTVRGRFTISRDNAKN | |
| cleavable (scFv | SLYLQMNSLRAEDTAVYYCARDSNWDALDYWGQGTT | |
| mask-IL2v- | VTVSSGGGGSGGGGGGSGGGGSGGGDIQMTQSPSSLSA | |
| Fc knob) | SVGDRVTITCKASQNVGTNVGWYQQKPGKAPKALIYS | |
| | ASFRYSGVPSRFSGSGSGTDFTLTISSLQPEDFATYYCQQ | |
| | YYTYPYTFGGGTKVEIKGGGGSGGGGSGGGGSGGGGS | |
| | GGGGSGGGGSGGGGSGGGAPASSSTKKTQLQLEHLLL | |
| | DLQMILNGINNYKNPKLTRMLTAKFAMPKKATELKHL | |
| | QCLEEELKPLEEVLNGAQSKNFHLRPRDLISNINVIVLEL | |
| | KGSETTFMCEYADETATIVEFLNRWITFAQSIISTLTGGG | |
| | GSGGGGSDKTHTCPPCPAPEAAGGPSVFLFPP | |
| | KPKDTLMISRTPEVTCVVVDVSHEDPEVKFNWYVDGV | |
| | EVHNAKTKPREEQYNSTYRVVSVLTVLHQDWLNGKEY | |
| | KCKVSNKALGAPIEKTISKAKGQPREPQVYTLPPCRDEL | |
| | TKNQVSLWCLVKGFYPSDIAVEWESNGQPENNYKTTPP | |
| | VLDSDGSFFLYSKLTVDKSRWQQGNVFSCSVMHEALH | |
| | NHYTQKSLSLSPGK | |
| CD8-IL2v | EVQLVQSGAEVKKPGASVKVSCKASGFNIKDTYIHWVR | 7 |
| MT204 1xMMP9/ | QAPGQGLEWIGRIDPANDNTLYASKFQGRATITADTSTS | |
| Matriptase | TAYLELSSLRSEDTAVYYCGRGYGYYVFDHWGQGTLV | |
| (HC hole) | TVSSASTKGPSVFPLAPSSKSTSGGTAALGCLVEDYFPEP | |
| | VTVSWNSGALTSGVHTFPAVLQSSGLYSLSSVVTVPSSS | |
| | LGTQTYICNVNHKPSNTKVDEKVEPKSCDKTHTCPPCP | |
| | APEAAGGPSVFLFPPKPKDTLMISRTPEVTCVVVDVSHE | |
| | DPEVKFNWYVDGVEVHNAKTKPREEQYNSTYRVVSVL | |
| | TVLHQDWLNGKEYKCKVSNKALGAPIEKTISKAKGQPR | |
| | EPQVCTLPPSRDELTKNQVSLSCAVKGFYPSDIAVEWES | |
| | NGQPENNYKTTPPVLDSDGSFFLVSKLTVDKSRWQQGN | |
| | VFSCSVMHEALHNHYTQKSLSLSPGK | |
| CD8-IL2v | EVQLVESGGGLVQPGGSLRLSCAASGFTFSSYTLAWVR | 8 |
| MT204 | QAPGKCLEWVAAIDSSSYTYSPDTVRGRFTISRDNAKNS | |
| 1xMMP9/ | LYLQMNSLRAEDTAVYYCARDSNWDALDYWGQGTTV | |
| Matriptase | TVSSGGGGSGGGGSGGGGSGGGGSDIQMTQSPSSLSAS | |
| (ds-scFv mask- | VGDRVTITCKASQNVGTNVGWYQQKPGKAPKALIYSA | |
| IL2v-Fc knob) | SFRYSGVPSRFSGSGSGTDFTLTISSLQPEDFATYYCQQY | |
| | YTYPYTFGCGTKVEIKGGGGSGGSGGSSVHMPLGFLGP | |
| | RQARVVNGGSGGSGGSGGSAPASSSTKKTQLQLEHLLL | |
| | DLQMILNGINNYKNPKLTRMLTAKFAMPKKATELKHL | |
| | QCLEEELKPLEEVLNGAQSKNFHLRPRDLISNINVIVLEL | |
| | KGSETTFMCEYADETATIVEFLNRWITFAQSIISTLTGGG | |
| | GSGGGGSDKTHTCPPCPAPEAAGGPSVFL | |
| | FPPKPKDTLMISRTPEVTCVVVDVSHEDPEVKFNWYVD | |
| | GVEVHNAKTKPREEQYNSTYRVVSVLTVLHQDWLNGK | |
| | EYKCKVSNKALGAPIEKTISKAKGQPREPQVYTLPPCRD | |
| | ELTKNQVSLWCLVKGFYPSDIAVEWESNGQPENNYKTT | |
| | PPVLDSDGSFFLYSKLTVDKSRWQQGNVFSCSVMHEAL | |
| | HNHYTQKSLSLSPGK | |
| MT204 scFv | EVQLVESGGGLVQPGGSLRLSCAASGFTFSSYTLAWVR | 9 |
| mask-IL2v | QAPGKGLEWVAAIDSSSYTYSPDTVRGRFTISRDNAKN | |
| with 2xPMAKK | SLYLQMNSLRAEDTAVYYCARDSNWDALDYWGQGTT | |
| release sites | VTVSSGGGGSGGPMAKKGGGGSGGGDIQMTQSPSSLS | |
| | ASVGDRVTITCKASQNVGTNVGWYQQKPGKAPKALIY | |
| | SASFRYSGVPSRFSGSGSGTDFTLTISSLQPEDFATYYCQ | |
| | QYYTYPYTFGGGTKVEIKGGGGSGGGGSPMAKKGGGG | |
| | SGGGGSGGGGSGGGGSGGGAPASSSTKKTQLQLEHLLL | |
| | DLQMILNGINNYKNPKLTRMLTAKFAMPKKATELKHL | |
| | QCLEEELKPLEEVLNGAQSKNFHLRPRDLISNINVIVLEL | |
| | KGSETTFMCEYADETATIVEFLNRWITFAQSIISTLT | |
| MT204 scFv | EVQLVESGGGLVQPGGSLRLSCAASGFTFSSYTLAWVR | 10 |
| mask-IL2v | QAPGKGLEWVAAIDSSSYTYSPDTVRGRFTISRDNAKN | |
| without protease | SLYLQMNSLRAEDTAVYYCARDSNWDALDYWGQGTT | |
| release sites | VTVSSGGGGSGGGGGGSGGGGSGGGDIQMTQSPSSLSA | |
| | SVGDRVTITCKASQNVGTNVGWYQQKPGKAPKALIYS | |
| | ASFRYSGVPSRFSGSGSGTDFTLTISSLQPEDFATYYCQQ | |
| | YYTYPYTFGGGTKVEIKGGGGSGGGGGGGGSGGGGS | |
| | GGGGSGGGGSGGGGSGGGAPASSSTKKTQLQLEHLLL | |
| | DLQMILNGINNYKNPKLTRMLTAKFAMPKKATELKHL | |
| | QCLEEELKPLEEVLNGAQSKNFHLRPRDLISNINVIVLEL | |
| | KGSETTFMCEYADETATIVEFLNRWITFAQSIISTLT | |
| MT204 ds-scFv | EVQLVESGGGLVQPGGSLRLSCAASGFTFSSYTLAWVR | 11 |
| mask-IL2v | QAPGKCLEWVAAIDSSSYTYSPDTVRGRFTISRDNAKNS | |
| with 1xMMP9/ | LYLQMNSLRAEDTAVYYCARDSNWDALDYWGQGTTV | |
| Matriptase | TVSSGGGGSGGGGSGGGGSGGGGSDIQMTQSPSSLSAS | |
| release site | VGDRVTITCKASQNVGTNVGWYQQKPGKAPKALIYSA | |
| | SFRYSGVPSRFSGSGSGTDFTLTISSLQPEDFATYYCQQY | |
| | YTYPYTFGCGTKVEIKGGGGSGGSGGSSVHMPLGFLGP | |
| | RQARVVNGGSGGSGGSGGSAPASSSTKKTQLQLEHLLL | |
| | DLQMILNGINNYKNPKLTRMLTAKFAMPKKATELKHL | |
| | QCLEEELKPLEEVLNGAQSKNFHLRPRDLISNINVIVLEL | |
| | KGSETTFMCEYADETATIVEFLNRWITFAQSIISTLT | |
| MT204 scFv | EVQLVESGGGLVQPGGSLRLSCAASGFTFSSYTLAWVR | 12 |
| mask with | QAPGKGLEWVAAIDSSSYTYSPDTVRGRFTISRDNAKN | |
| 2xPMAKK | SLYLQMNSLRAEDTAVYYCARDSNWDALDYWGQGTT | |
| release sites | VTVSSGGGGSGGPMAKKGGGGSGGGDIQMTQSPSSLS | |
| | ASVGDRVTITCKASQNVGTNVGWYQQKPGKAPKALIY | |
| | SASFRYSGVPSRFSGSGSGTDFTLTISSLQPEDFATYYCQ | |
| | QYYTYPYTFGGGTKVEIKGGGGSGGGGSPMAKKGGGG | |
| | SGGGGSGGGGSGGGGSGGG | |
| human IL-2 | APTSSSTKKTQLQLEHLLLDLQMILNGINNYKNPKLTR | 13 |
| wild type | MLTFKFYMPKKATELKHLQCLEEELKPLEEVLNLAQSK | |
| | NFHLRPRDLISNINVIVLELKGSETTFMCEYADETATIVE | |
| | FLNRWITFCQSIISTLT | |
| IL2v CD8v11 | APASSSTKKTQLQLEHLLLDLQMILNGINNYKNPKLTR | 14 |
| Fc(kih) (IL2v- | MLTAKFAMPKKATELKHLQCLEEELKPLEEVLNGAQS | |
| Fc knob) | KNFHLRPRDLISNINVIVLELKGSETTFMCEYADETATIV | |
| | EFLNRWITFAQSIISTLTGGGGSGGGGSDKTHTCPPCPAP | |
| | EAAGGPSVFLFPPKPKDTLMISRTPEVTCVVVDVSHEDP | |
| | EVKFNWYVDGVEVHNAKTKPREEQYNSTYRVVSVLTV | |
| | LHQDWLNGKEYKCKVSNKALGAPIEKTISKAKGQPREP | |
| | QVYTLPPCRDELTKNQVSLWCLVKGFYPSDIAVEWESN | |
| | GQPENNYKTTPPVLDSDGSFFLYSKLTVDKSRWQQGNV | |
| | FSCSVMHEALHNHYTQKSLSLSPGK | |
| MMP Protease | GGGGSGGGGSGPLGLWSQGGGGSGGGGSGGGGSGG | 15 |
| linker | | |
| Protease | RQARVVNG | 16 |
| recognition site | | |
| 1 | | |
| Protease | VHMPLGFLGPGRSRGSFP | 17 |
| recognition site | | |
| 2 | | |
| Protease | RQARVVNGXXXXXVPLSLYSG | 18 |
| recognition site | | |
| 3 | | |
| Protease | RQARVVNGVPLSLYSG | 19 |
| recognition site | | |
| 4 | | |
| Protease | PLGLWSQ | 20 |
| recognition site | | |
| 5 | | |
| Protease | VHMPLGFLGPRQARVVNG | 21 |
| recognition site | | |
| 6 | | |
| Protease | FVGGTG | 22 |
| recognition site | | |
| 7 | | |
| Protease | KKAAPVNG | 23 |
| recognition site | | |
| 8 | | |
| Protease | PMAKKVNG | 24 |
| recognition site | | |
| 9 | | |
| Protease | QARAKVNG | 25 |
| recognition site | | |
| 10 | | |
| Protease | VHMPLGFLGP | 26 |
| recognition site | | |
| 11 | | |
| Protease | QARAK | 27 |
| recognition site | | |
| 12 | | |
| Protease | VHMPLGFLGPPMAKK | 28 |
| recognition site | | |
| 13 | | |
| Protease | KKAAP | 29 |
| recognition site | | |
| 14 | | |
| Protease | PMAKK | 30 |
| recognition site | | |
| 15 | | |
| Protease | YAARKGGI | 31 |
| recognition site | | |
| 16 | | |
| Protease | PQARK | 32 |
| recognition site | | |
| 17 | | |
| Protease | HQARK | 33 |
| recognition site | | |
| 18 | | |

### Further Aspects of the Invention

In a further aspect, the invention provides an mask comprising (i) a masking moiety and (ii) a linker comprising a first protease cleavage site, wherein the masking moiety is covalently attachable to an IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site.

In one embodiment, the masking moiety is covalently attachable to the amino-terminus or the carboxy-terminus of an IL-2 polypeptide through the linker. In one embodiment, the masking moiety is an IL-2 antagonist. In one embodiment, the masking moiety is an IL-2 antibody or an IL-2 receptor subunit. In one embodiment, the IL-2 antibody comprises a Fab molecule. In one embodiment, the masking moiety is derived from MT204. In one embodiment, the masking moiety is MT204. The MT204 antibody is disclosed e.g. in Volkland et al., Molecular Immunology 44 (2007) 1743-1753, and PCT-publication WO 2006/128690 A1. In one embodiment, the Fab molecule is a single-chain Fab molecule. In one embodiment, the second protease cleavage site is located between the variable domain of the heavy chain (VH) and the variable domain of the light chain (VL) of the Fab. In one embodiment, the first protease cleavage site and the second protease cleavage site each comprise at least one protease recognition sequence. In one embodiment, the protease recognition sequence of the first protease cleavage site and/or the protease recognition sequence of the second protease cleavage site (recognition sequence) is selected from the group consisting of: (a) RQARVVNG (SEQ ID NO: 16); (b) VHMPLGFLGPGRSRGSFP (SEQ ID NO:17); (c) RQARVVNGXXXXXVPLSLYSG (SEQ ID NO:18), wherein X is any amino acid; (d) RQARVVNGVPLSLYSG (SEQ ID NO:19); (e) PLGLWSQ (SEQ ID NO:20); (f) VHMPLGFLGPRQARVVNG (SEQ ID NO:21); (g) FVGGTG (SEQ ID NO:22); (h) KKAAPVNG (SEQ ID NO:23); (i) PMAKKVNG (SEQ ID NO:24); (j) QARAKVNG (SEQ ID NO: 25); (k) VHMPLGFLGP (SEQ ID NO:26); (l) QARAK (SEQ ID NO:27); (m) VHMPLGFLGPPMAKK (SEQ ID NO:28); (n) KKAAP (SEQ ID NO:29); (0) PMAKK (SEQ ID NO: 30); (p) YAARKGGI (SEQ ID NO:31); (q) PQARK (SEQ ID NO:32); and (r) HQARK (SEQ ID NO:33).

In one embodiment, the protease recognition sequence of the first protease cleavage site is different from the protease recognition sequence of the second protease cleavage site. In one embodiment, the protease recognition sequence of the first protease cleavage site is the same as the protease recognition sequence of the second protease cleavage site.

In one embodiment, the protease recognition sequence of the first protease cleavage site and/or the protease recognition sequence of the second protease cleavage site is PMAKK (SEQ ID NO: 30). In one embodiment, the protease recognition sequence of the first protease cleavage site is PMAKK (SEQ ID NO:30). In one embodiment, the protease recognition sequence of the second protease cleavage site is PMAKK (SEQ ID NO:30). In one embodiment, the protease recognition sequence of the first protease cleavage site and the protease recognition sequence of the second protease cleavage site is PMAKK (SEQ ID NO:30).

In one embodiment, the IL-2 polypeptide is a wild-type IL-2, preferably a human IL-2 according to SEQ ID NO: 13, or a mutant IL-2 polypeptide. In one embodiment, the mutant IL-2 polypeptide comprises any amino acid substitution selected from the group T3A, F42A, Y45A, L72G, C125A of human IL-2 according to SEQ ID NO:13. In one embodiment, the mutant IL-2 polypeptide comprises the amino acid substitutions F42A, Y45A and L72G of human IL-2 according to SEQ ID NO:13. In one embodiment, the mutant IL-2 polypeptide comprises the amino acid substitutions T3A, F42A, Y45A, L72G and C125A of human IL-2 according to SEQ ID NO: 13.

In one embodiment, the mask comprising the masking moiety and the linker comprises the amino acid sequence of SEQ ID NO: 12.

### Further Aspects of the Disclosure

1. A protease-activatable interleukin-2 (IL-2) polypeptide comprising (i) an IL-2 polypeptide, (ii) a masking moiety and (iii) a linker comprising a first protease cleavage site, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site.

2. The protease-activatable IL-2polypeptide of aspect 1, wherein the masking moiety is covalently attached to the amino-terminus or carboxy-terminus of the interleukin-2 polypeptide through the linker.

3. The protease-activatable IL-2polypeptide of aspect 1 or 2, wherein the masking moiety is an IL-2 antagonist.

4. The protease-activatable IL-2polypeptide of any of aspects 1 to 3, wherein the masking moiety is an IL-2 antibody or an IL-2 receptor subunit.

5. The protease-activatable IL-2polypeptide of aspect 4, wherein the IL-2 antibody comprises a Fab molecule.

6. The protease-activatable IL-2polypeptide of any of aspects 1 to 4, wherein the masking moiety is an antibody derived from MT204, preferably MT204.

6. The protease-activatable IL-2 polypeptide of aspect 5 or 6, wherein the Fab molecule is a single-chain Fab molecule.

7. The protease-activatable IL-2 polypeptide of aspect 6, wherein the second protease cleavage site is located between the variable domain of the heavy chain (VH) and the variable domain of the light chain (VL) of the Fab.

8. The protease-activatable IL-2 polypeptide of any of aspects 1-7, wherein the first protease cleavage site and the second protease cleavage site each comprise at least one protease recognition sequence.

9. The protease-activatable IL-2 polypeptide of any one of aspects 1-8, wherein the protease recognition sequence of the first protease cleavage site and/or the protease recognition sequence of the second protease cleavage site is selected from the group consisting of:
(a) RQARVVNG according to SEQ ID NO:16;
(b) VHMPLGFLGPGRSRGSFP according to SEQ ID NO:17;
(c) RQARVVNGXXXXXVPLSLYSG according to SEQ ID NO:18, wherein X is any amino acid;
(d) RQARVVNGVPLSLYSG according to SEQ ID NO:19;
(e) PLGLWSQ according to SEQ ID NO:20;
(f) VHMPLGFLGPRQARVVNG according to SEQ ID NO:21;
(g) FVGGTG according to SEQ ID NO:22;
(h) KKAAPVNG according to SEQ ID NO:23;
(i) PMAKKVNG according to SEQ ID NO:24;
(j) QARAKVNG according to SEQ ID NO:25;
(k) VHMPLGFLGP according to SEQ ID NO:26;
(l) QARAK according to SEQ ID NO:27;
(m) VHMPLGFLGPPMAKK according to SEQ ID NO:28;
(n) KKAAP according to SEQ ID NO:29;
(o) PMAKK according to SEQ ID NO:30;
(p) YAARKGGI according to SEQ ID NO:31;
(q) PQARK according to SEQ ID NO:32; and
(r) HQARK according to SEQ ID NO:33.

10. The protease-activatable IL-2 polypeptide of aspect 8 or 9, wherein the protease recognition sequence of the first protease cleavage site and/or the protease recognition sequence of the second protease cleavage site is PMAKK (SEQ ID NO:30).

11. The protease-activatable IL-2 polypeptide of any of aspects 1 to 11, wherein the IL-2 polypeptide is a wild-type IL-2, preferably a human IL-2 according to SEQ ID NO: 13, or a mutant IL-2 polypeptide.

12. The protease-activatable IL-2 polypeptide of aspect 11, wherein the mutant IL-2 polypeptide comprises any amino acid substitution selected from the group T3A, F42A, Y45A, L72G, C125A of human IL-2 according to SEQ ID NO:13.

13. The protease-activatable IL-2 polypeptide of aspect 11 or 12, wherein the mutant IL-2 polypeptide comprises the amino acid substitutions F42A, Y45A and L72G of human IL-2 according to SEQ ID NO:13.

14. The protease-activatable IL-2 polypeptide of any of aspects 11 to 13, wherein the mutant IL-2 polypeptide comprises the amino acid substitutions T3A, F42A, Y45A, L72G and C125A of human IL-2 according to SEQ ID NO: 13 15. The protease-activatable IL-2 polypeptide of any of aspects 11 to 14, wherein the masking moiety and the linker comprise the amino acid sequence of SEQ ID NO: 12.

16. The protease-activatable IL-2 polypeptide of any of aspects 11 to 15, wherein the protease activatable IL-2 polypeptide comprises the amino acid sequence of SEQ ID NO: 9.

17. The protease-activatable IL-2 polypeptide of any one of aspects 1 to 16, wherein the IL-2 polypeptide is further attached to a non-IL-2 moiety.

18. The protease-activatable IL-2 polypeptide of aspect 17, wherein said IL-2 polypeptide shares a carboxy-terminal peptide bond with the masking moiety and an amino-terminal peptide bond with the non-IL-2 moiety or wherein said IL-2 polypeptide shares a amino-terminal peptide bond with the masking moiety and an carboxy-terminal peptide bond with the non-IL-2 moiety.

19. The protease-activatable IL-2 polypeptide of aspects 17 or 18, wherein said non-IL-2 moiety is an antigen binding moiety or an effector cell binding moiety.

20. An immunoconjugate comprising a protease-activatable IL-2 polypeptide of any one of aspects 1 to 16 and an antigen binding moiety and/or an effector cell binding moiety.

21. The immunoconjugate of aspect 20, wherein said protease-activatable IL-2 polypeptide shares an amino- or carboxy-terminal peptide bond with the antigen binding moiety or the effector cell binding moiety.

22. The immunoconjugate of aspects 20 or 21, wherein said immunoconjugate comprises a first and a second antigen binding moiety or a first and a second effector cell antigen binding moiety or an antigen binding moiety and an effector cell binding moiety.

23. The immunoconjugate of aspect 22, (i) wherein said protease-activatable IL-2 polypeptide shares an amino- or carboxy-terminal peptide bond with said first antigen binding moiety and said second antigen binding moiety shares an amino- or carboxy-terminal peptide bond with either a) said protease-activatable IL-2 polypeptide or b) said first antigen binding moiety; (ii) wherein said protease-activatable IL-2 polypeptide shares an amino- or carboxy-terminal peptide bond with said first effector cell binding moiety and said second effector cell binding moiety shares an amino- or carboxy-terminal peptide bond with either a) said protease-activatable IL-2 polypeptide or b) said first effector cell binding moiety; (iii) wherein said protease-activatable IL-2 polypeptide shares an amino- or carboxy-terminal peptide bond with the antigen binding moiety and the effector cell binding moiety shares an amino- or carboxy-terminal peptide bond with either a) said protease-activatable IL-2 polypeptide or b) said antigen binding moiety; or (iv) wherein said protease-activatable IL-2 polypeptide shares an amino- or carboxy-terminal peptide bond with the effector cell binding moiety and the antigen binding moiety shares an amino- or carboxy-terminal peptide bond with either a) said protease-activatable IL-2 polypeptide or b) said effector cell binding moiety.

24. The protease-activatable IL-2 polypeptide of aspect 17 or the immunoconjugate of any one of claims 20 to 23, wherein said antigen binding moiety or effector cell binding moiety is an antibody or an antibody fragment.

25. The protease-activatable IL-2 polypeptide of aspect 19 or the immunoconjugate of any one of aspects 20 to 24, wherein said antigen binding moiety and/or said effector cell binding moiety is selected from a Fab molecule and a scFv molecule.

26. The protease-activatable IL-2 polypeptide of aspect 19 or the immunoconjugate of any one of aspects 20 to 25, wherein said antigen binding moiety and/or said effector cell binding moiety is an immunoglobulin molecule, particularly an IgG molecule.

27. The mutant IL-2 polypeptide of aspect 19 or the immunoconjugate of any one of aspects 20 to 26, wherein said antigen binding moiety is directed to an antigen presented on a tumor cell or in a tumor cell environment and/or wherein said effector cell binding moiety is directed to an effector cell present in a tumor cell environment in order to achieve cis-targeting.

28. An isolated polynucleotide encoding the protease-activatable IL-2 polypeptide or immunoconjugate of any one of aspects 1 to 27.

29. An expression vector comprising the polynucleotide of aspect 28.

30. A host cell comprising the polynucleotide of aspect 28 or the expression vector of aspect 29.

31. A method of producing a protease-activatable IL-2 polypeptide or an immunoconjugate thereof, comprising culturing the host cell of aspect 30 under conditions suitable for the expression of the protease-activatable IL-2 polypeptide or the immunoconjugate.

32. A protease-activatable IL-2 polypeptide or immunoconjugate produced by the method of aspect 31.

33. A pharmaceutical composition comprising the protease-activatable IL-2 polypeptide or immunoconjugate of any one of aspects 1 to 27 or 32 and a pharmaceutically acceptable carrier.

34. The protease-activatable IL-2 polypeptide or immunoconjugate of any one of aspects 1 to 27 or 32 for use in the treatment of a disease in an individual in need thereof.

35. The protease-activatable IL-2 polypeptide or immunoconjugate of aspect 34 wherein said disease is cancer.

36. Use of the protease-activatable IL-2 polypeptide or immunoconjugate of any one of aspects 1 to 27 or 32 for manufacture of a medicament for treating a disease in an individual in need thereof.

37. A method of treating disease in an individual, comprising administering to said individual a therapeutically effective amount of a composition comprising the protease-activatable IL-2 polypeptide or immunoconjugate of any one of aspects 1 to 27 or 32 in a pharmaceutically acceptable form.

38. The method of aspect 37, wherein said disease is cancer.

39. A method of stimulating the immune system of an individual, comprising administering to said individual an effective amount of a composition comprising the protease-activatable IL-2 polypeptide or immunoconjugate of any one of aspects 1 to 27 or 32 in a pharmaceutically acceptable form.

### EXAMPLES

The following are examples of methods and compositions of the invention. It is understood that various other embodiments may be practiced, given the general description provided above.

### Example 1

### Generation of Targeted and Masked Antibody IL2v Fusions (with scFv Mask) and Control Constructs

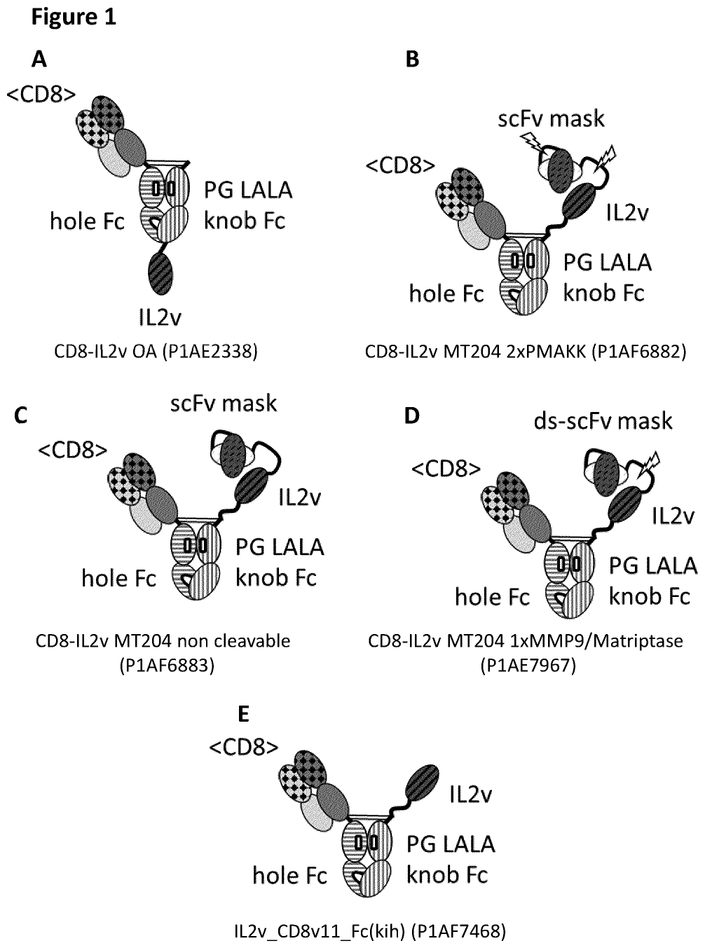

One-armed CD8-targeted IL2v fusion constructs were generated. They bind monovalently to human CD8 via the N-terminal Fab fragment on the Fc hole chain and are heterodimerized with an Fc knob chain that either carries a non-masked C-terminal IL2v (non-masked control construct, SEQ ID NOs 1, 2, 3; FIG. 1A) or a non-masked N-terminal IL2v (SEQ ID NOs 4, 2, 14; FIG. 1E). Heterodimerization was achieved by application of knobs-into-holes technology and binding to activating human Fcγ receptors as well as complement component C1q has been abrogated by introduction of PG LALA mutations in the Fc portion of the antibodies. Three different CD8-targeted masked IL2v constructs were generated. The first construct comprises a scFv mask that carries two PMAKK matriptase recognition sites, wherein one PMAKK matriptase recognition site is positioned in the linker between the VH and VL of the scFv mask and one in the linker between the scFv mask and the IL2v (SEQ ID NOs 4, 2, 5, FIG. 1B). The second construct comprises a scFv mask that carries no protease recognition sites at all (non-cleavable control; SEQ ID NOs 4, 2, 6; FIG. 1C). The third construct comprises a disulfide-stabilized scFv mask that carries one MMP9/Matriptase recognition site between the scFv mask and the IL2v (SEQ ID NOs 1, 7, 8; FIG. 1D). These constructs are schematically depicted in FIGS. 1A-E.

### Production of Masked Antibody IL2v Fusions and Control Constructs

Masked antibody IL2v fusions and control constructs were generated by transient transfection of Expi293F cells. Cells were seeded in Expi293 media (Gibco, Cat. No 1435101) at a density of 2.5×10e6/ml. Expression vectors and ExpiFectamine (Gibco, ExpiFectamine transfection kit, Cat. No 13385544) were separately mixed in OptiMEM (Gibco, Cat. No 11520386). After 5 minutes, both solutions were combined, mixed by pipetting and incubated for 25 minutes at room temperature. Cells were added to the vector/ExpiFectamine solution and incubated for 24 hours at 37° C. in a shaking incubator with a 5% CO₂ atmosphere. One day post transfection, supplements (Enhancer 1+2, ExpiFectamine transfection kit) were added. Cell supernatants were harvested after 4-5 days by centrifugation and subsequent filtration (0.2 μm filter).

### Purification and Analytics of Masked Antibody IL2v Fusions and Control Constructs

Proteins were purified from filtered cell culture supernatants according to standard protocols. In brief, the fusion proteins were purified from cell culture supernatants by a combination of protein A-affinity chromatography using Protein A MabSelect SuRe (equilibration buffer: 20 mM sodium citrate, 20 mM sodium phosphate, pH 7.5; elution buffer: 20 mM sodium citrate, pH 3.0) and cation exchange chromatography (cIEX) using a POROS XS column (20 mM NaPhosphate (0-450 mM NaCl gradient) pH7.1). The protein was concentrated by centrifugation (MWCO 30.000; Amicon Ultra, Millipore), and aggregated protein was separated from monomeric protein by preparative size exclusion chromatography and formulated into 20 mM histidine, 140 mM sodium chloride, with or without 0.01% Tween20, pH 6.0.

Alternatively, the construct comprising a scFv mask that carries one MMP9/Matriptase recognition site between the scFv mask and IL2v (CD8-IL2v MT204 1×MMP9/Matriptase; SEQ ID NOs 1, 7, 8), has been purified by protein A-affinity chromatography using Protein A MabSelect SuRe (equilibration buffer: 1×PBS, pH 7.4; elution buffer: 50 mM sodium citrate, pH 3.0), was neutralized and concentrated by centrifugation (MWCO 30.000; Amicon Ultra, Millipore). Aggregated protein was separated from monomeric protein by preparative size exclusion chromatography (HiLoad 16/60 Superdex 200) with 20 mM histidine, 140 mM sodium chloride, pH 6.0 as running buffer and subsequently concentrated again by centrifugation (MWCO 30.000; Amicon Ultra, Millipore).

The concentrations of the purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of monomer content was performed by HPLC chromatography at 25° C. using analytical size-exclusion chromatography (TSKgel G3000 SW XL or BioSuite High Resolution SEC) equilibrated in running buffer (200 mM arginine, 25 mM K2HPO4, 125 mM NaCl, 0.02% NaN3, pH 6.7, or 200 mM K2HPO4/KH2PO4, 250 mM KCl pH7.0, respectively).

### Example 2

### Proliferation of NK92 Cells Upon Treatment with Masked CD8-IL2v Constructs after Digestion with Matriptase

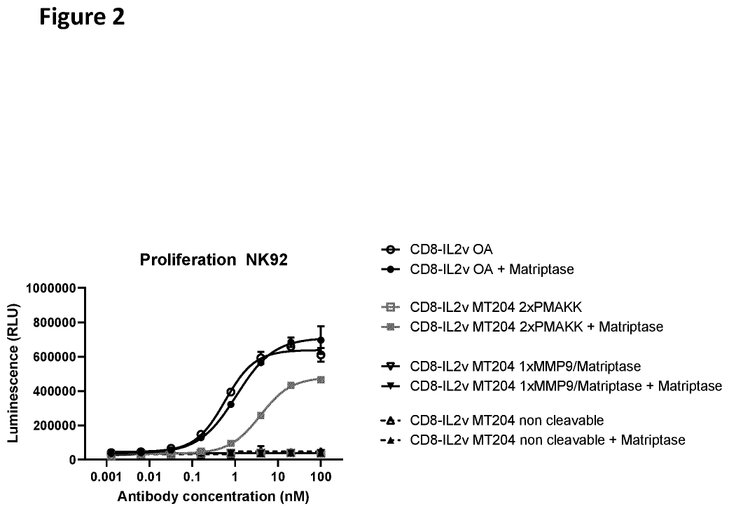

The proliferation of the human NK cell line NK92 was assessed upon treatment for four days with MT204-masked CD8-IL2v constructs containing two PMAKK linkers or one MMP9/Matriptase linker and compared to the activity of the unmasked CD8-IL2v OA (one-armed) and the CD8-IL2v MT204 non cleavable construct after digestion with Matriptase or undigested. The CD8-IL2v MT204 2×PMAKK induced proliferation after digestion with Matriptase but did not induce any proliferation when the linker was not digested by Matriptase (FIG. 2). In contrast, the CD8-IL2v MT204 1×MMP9/Matriptase induced no proliferation after digestion with Matriptase (FIG. 2).

NK92 cells were harvested, counted and assessed for viability. Cells were washed three times with PBS to remove residual IL2. The washed NK92 cells were re-suspended in fresh medium (advanced RPMI1640, 2% FCS, 1% Glutamine) without IL2 to 160′000 cells per ml and 12.5 μl of the cell suspension was transferred in a 384-well cell culture treated flat bottom plate. 30 μg of the antibody-cytokine fusions were digested with 6 μl Matriptase (Enzo ˜2.5 U/μl (Matriptase: human recombinant Matriptase from Enzo, ALX-201-246-U250) or without Matriptase as undigested control) for 2 hours at 37° C. in 60 μl Matriptase buffer (50 mM Tris, 50 mM NaCl, 0.01% Tween 20, pH 9.0) and 12.5 μl of the diluted antibodies were added after digestion per well to reach a final volume of 25 μl per well. The plate was incubated for 4 days in the incubator.

After four days, the CellTiter-Glo (Promega) reagents and the cell culture plate were equilibrated to room temperature. The CellTiter-Glo solution was prepared as described in the manufacturer's instructions and 25 μl of the solution were added to each well. After 10 min of incubation, remaining aggregates were re-suspended by pipetting and 40 μl of the mixture were transferred to a white flat bottom plate. The luminescence was measured with a Tecan Spark 10M multimode reader.

FIG. 2 shows the proliferation of NK92 cells induced by CD8-IL2v MT204 2×PMAKK compared to CD8-IL2v OA, CD8-IL2v MT204 1×MMP9/Matriptase and the CD8-IL2v MT204 non-cleavable construct after digestion with Matriptase or undigested. Proliferation was measured after 4 days. Proliferation of masked CD8-IL2v MT204 2×PMAKK was induced after digestion with Matriptase. No proliferation was induced by CD8-IL2v 1×MMP9/Matriptase after digestion with Matriptase indicating that a protease release site in the scFv linker is required for unmasking and subsequent activation.

### Example 3

### Proliferation and Activation of PBMCs Upon Treatment with Masked CD8-IL2v Constructs after Digestion with Matriptase

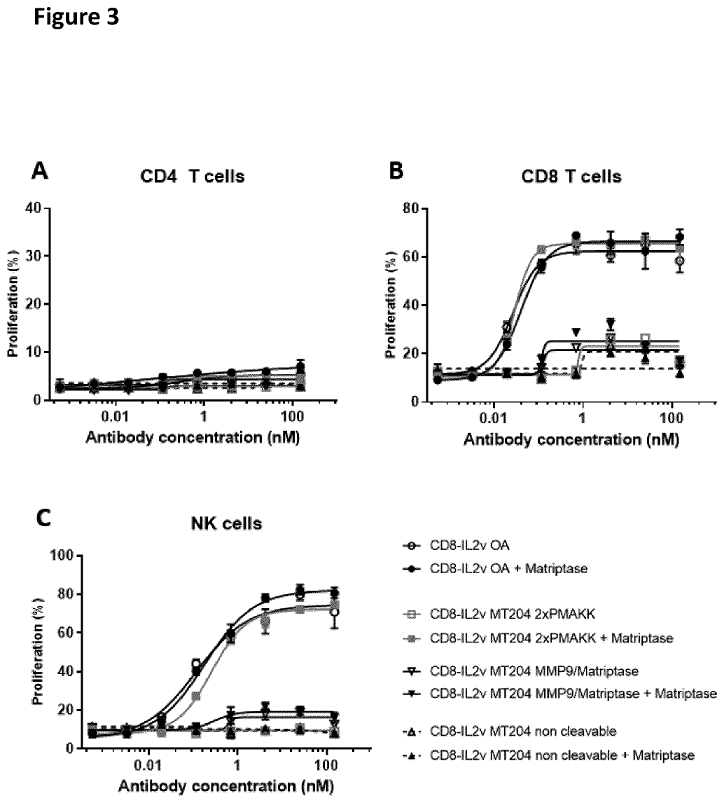

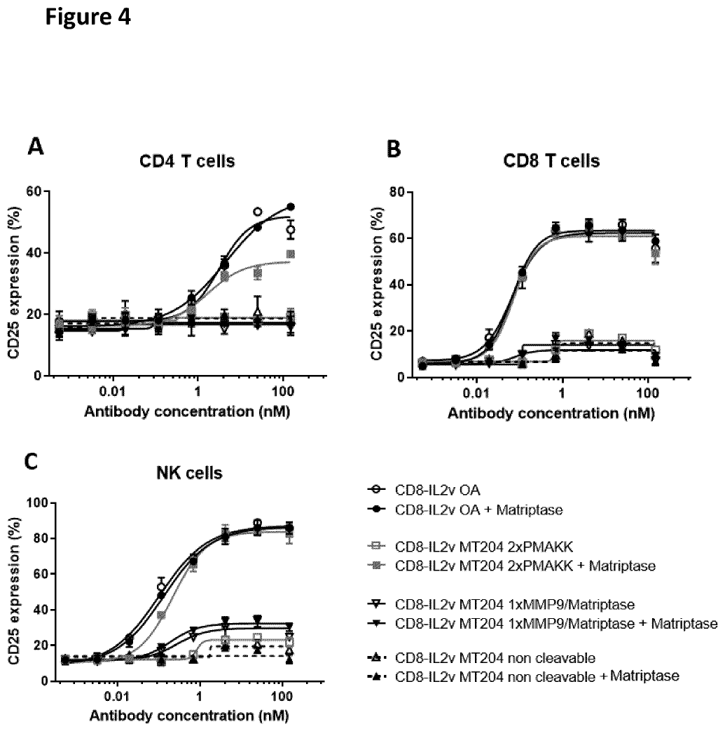

Next, the masked CD8-IL2v constructs were tested for their activity on PBMCs and compared to the unmasked CD8-IL2v (positive control) and the masked non-cleavable CD8-IL2v (negative control). After five days of treatment with the constructs, proliferation of CD8 T cells, CD4 T cells and NK cells (FIGS. 3A-C) and CD25 upregulation (FIGS. 4A-C) as a marker of activation of CD8 T cells, NK cells and CD4 T cells, was measured by flow cytometry. After cleavage with Matriptase, the CD8-IL2v MT204 2×PMAKK induced comparable proliferation and activation to the unmasked CD8-IL2v on CD8 T cells and NK cells. The CD8-IL2v MT204 1×MMP9/Matriptase induced no proliferation and activation of CD4 T cells, CD8 T cells and NK cells after digestion with Matriptase.

Freshly isolated PBMCs from healthy donors were labeled with CFSE (5 (6)-Carboxyfluorescein diacetate N-succinimidyl ester, 21888, Sigma-Aldrich). Briefly, PBMCs were washed once with PBS. In parallel, the CSFE stock solution (2 mM in DMSO) was diluted 1:20 in PBS. PBMCs were resuspended in prewarmed PBS to 1 Mio/ml, 1 ml of the CFSE solution was added to 1 ml cell suspension and the cells were mixed immediately. For an optimal labeling, the cells were incubated for 15 min at 37° C. Then 10 ml prewarmed medium (RPMI1640, 10% FCS, 1% Glutamine) were added to stop the labeling reaction. The cells were spun down for 10 min at 400 g and resuspended in fresh medium to 1 Mio/ml and incubated for additional 30 min at 37° C. Finally, the cells were washed once with medium and resuspended in fresh medium. 50 μl of the labeled PBMCs were seeded in a 96 well round bottom plate (100′000 cells per well). In parallel, 40 ug of antibody-cytokine fusions were digested with 8 μl Matriptase (˜2.5 U/μl, (human recombinant Matriptase from Enzo, ALX-201-246-U250)) or incubated without Matriptase as undigested control for 2 hours at 37° C. in 80 μl Matriptase buffer (50 mM Tris, 50 mM NaCl, 0.01% Tween 20, pH 9.0). 50 μl of the indicated molecules were transferred per well and 100 μl of medium were added per well to reach a final volume of 200 μl/well. After five days of incubation, the cells were washed once with FACS buffer and stained with 30 μl of a mixture of anti-human CD3 BUV359 (563546, BD), anti-human CD4 PE (300539, Biolegend), anti-human CD8 APC (344722, BioLegend), anti-human CD56 BV421 (318328, BioLegend) and CD25 PE/Cy7 (302612, BioLegend) in FACS buffer for 30 min at 4° C. Afterwards, PBMCs were washed twice with FACS buffer before fixing them with 2% PFA in FACS buffer and measuring the fluorescence with a BD Fortessa. Proliferation was determined by measuring CFSE dilution of CD8 T cells (CD3+CD8+), CD4 T cells (CD3+CD4+) and NK cells (CD3-CD56+) and activation was determined by CD25 upregulation on CD8 T cells, CD4 T cells and NK cells.

FIG. 3A-C show the proliferation of CD4 T cells, CD8 T cells and NK cells within PBMCs upon treatment for 5 days with Matriptase-digested or non-digested CD8-IL2v MT204 2×PMAKK, CD8-IL2v MT204 1×MMP9/Matriptase, CD8-IL2v OA or CD8-IL2v MT204 non cleavable determined by flow cytometry. CFSE dye dilution was used as indicator for proliferation. Comparable proliferation to the unmasked CD8-IL2v was induced by CD8-IL2v MT204 2×PMAKK after Matriptase digestion on CD8 T cells and NK cells. No proliferation was induced by CD8-IL2v 1×MMP9/Matriptase and CD8-IL2v MT204 non cleavable after digestion with Matriptase.

FIG. 4A-C show the activation of CD4 T cells, CD8 T cells and NK cells within PBMCs upon treatment for 5 days with Matriptase digested or non-digested CD8-IL2v MT204 2×PMAKK, CD8-IL2v MT204 1×MMP9/Matriptase, CD8-IL2v OA or CD8-IL2v MT204 non-cleavable determined by flow cytometry. CD25 expression on NK cells, CD4 T cells and CD8 T cells was used as a marker of activation. Comparable activation to the unmasked CD8-IL2v was induced by CD8-IL2v MT204 2×PMAKK after Matriptase digestion on CD8 T cells and NK cells. No activation was induced by CD8-IL2v 1×MMP9/Matriptase and CD8-IL2v MT204 non cleavable after digestion with Matriptase.

### Example 4

### Matriptase Digestion of Masked Constructs and CE-SDS Analysis of Digested Probes

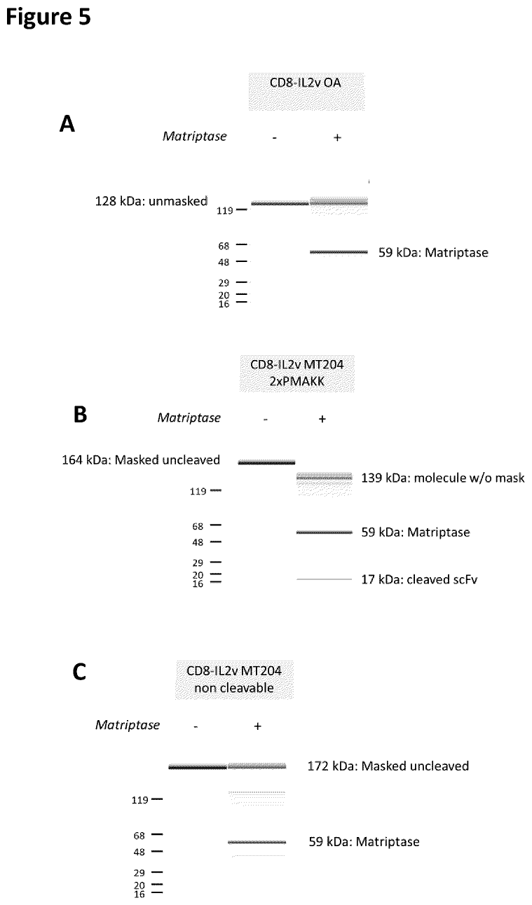

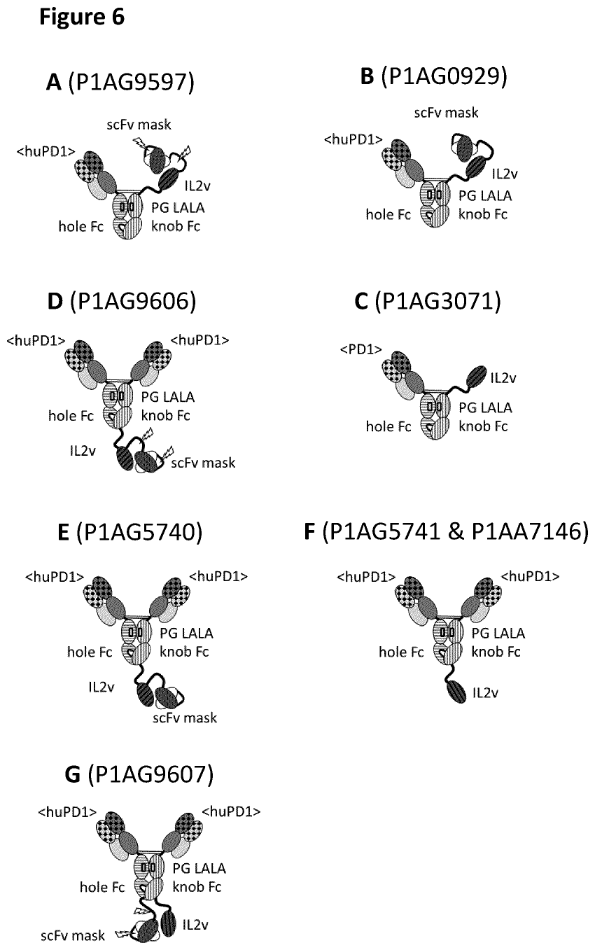

Masked constructs were incubated for 2 h at 37° with Matriptase before CE-SDS analysis. Purity and molecular weight of the proteins were analyzed by CE-SDS in the presence and absence of a reducing agent using a LabChip GX Touch (Perkin Elmer) following the manufacturer instructions. The molecular weights for the non-reduced probes were recorded and are reported in Table 1, and the electronical gels are shown in FIG. 5A-C.

TABLE 1
Expected and measured molecular weight of constructs
incubated with and without matriptase for 2 h at
37° C. determined by non-reduced CE-SDS analysis.

| | Expected | Measured | |
|---|---|---|---|
| | molecular | molecular | |
| Construct | weight | weight | Comment |
| CD8-IL2v OA | 114 kDa | 128 kDa as in | intact protein |
| (P1AE2338-003) + | | FIG. 5A | |
| matriptase | | | |
| CD8-IL2v OA | 114 kDa | 127 kDa as in | intact protein |
| (P1AE2338-003) | | FIG. 5A | |
| CD8-IL2v MT204 | 115 kDa + 14 | 139 kDa + | digestion |
| 2xPMAKK | kDa + 13 kDa | 17 kDa as in | complete (no |
| (P1AF6882-006) + | | FIG. 5B | band at 164 |
| matriptase | | | kDa observed) |
| CD8-IL2v MT204 | 142 kDa | 164 kDa as in | intact protein |
| 2xPMAKK | | FIG. 5B | |
| (P1AF6882-006) | | | |
| CD8-IL2v MT204 | 141 kDa | 172 kDa as in | intact protein |
| non cleav | | FIG. 5C | |
| (P1AF6883-007) + | | | |
| matriptase | | | |
| CD8-IL2v MT204 | 141 kDa | 172 kDa as in | intact protein |
| non cleavable | | FIG. 5C | |
| (P1AF6883-007) | | | |

The molecules analyzed in example 4 behave as expected when cleaved with matriptase. Incubation with matriptase results in the specific cleavage of the linker sequence. Unspecific cleavage was not observed for the constructs lacking a mask and constructs with a non-cleavable linker (Table 1, FIGS. 5A and 5C). The cleavable construct (containing two cleavage sites) is cleaved twice resulting in two halves of the scFv mask detected at 17 kDa and overlapping on CE-SDS (Table 1, FIG. 5B). Conclusively, matriptase is a specific enzyme that does not unspecifically cleave the tested constructs and cleaves only at both expected cleavage sites.

### Example 5A

### Generation of PD1-Targeted Masked IL2v Immunoconjugates with scFv Mask and Control Constructs

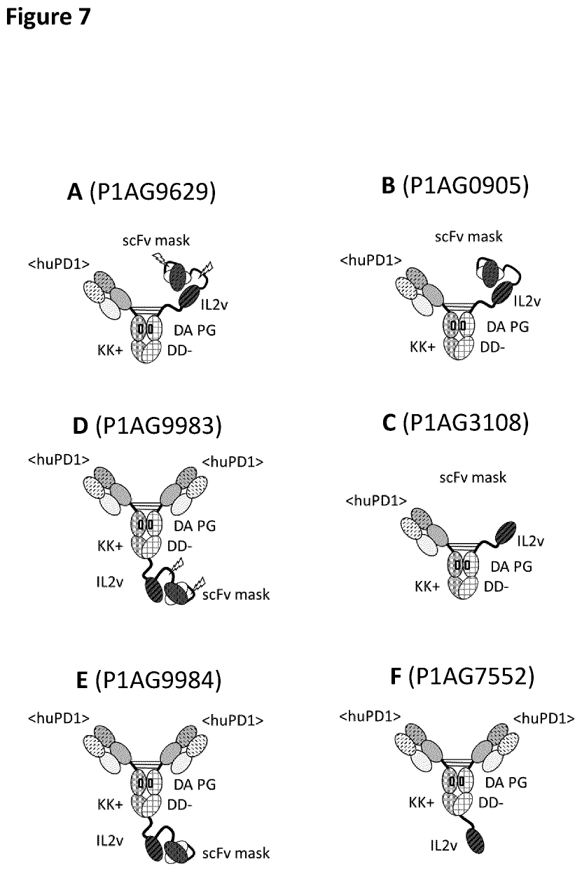

One-armed and bivalently human PD1-targeted IL2v immunoconjugates were generated. They bind monovalently or bivalently to human PD1 via the N-terminal Fab fragment(s) on the Fc hole chain (one-armed human PD1-targeted constructs) or on the Fc hole and Fc knob chain (bivalently human PD1-targeted constructs) whereas the Fc knob chain additionally carries a masked (matriptase cleavable or non-matriptase cleavable) or non-masked N- or C-terminal IL2v. The C-terminally masked IL2v constructs either carry IL2v and the mask ‘in-line’ on the same Fc knob chain or, alternatively, carry IL2v on the Fc knob chain and the mask on the Fc hole chain. Heterodimerization was achieved by application of knobs-into-holes technology and binding to activating human Fcγ receptors as well as complement component C1q has been abrogated by introduction of PG LALA mutations in the Fc portion of the antibodies. The matriptase-cleavable N- and C-terminally masked IL2v constructs carry two PQARK matriptase recognition sites, wherein one PQARK matriptase recognition site is positioned in the linker between the VH and VL domains of the scFv mask and another one in the linker between the scFv mask and the IL2v in the ‘in-line’ constructs or between the C-terminus of the Fc hole chain and the scFv mask in the construct with IL2v and mask on two separate heavy chains. Additionally, respective non-matriptase cleavable control constructs (devoid of PQARK matriptase recognition sites) and non-masked control constructs (devoid of the scFv mask) have been generated. These constructs are schematically depicted in FIGS. 6A-G.

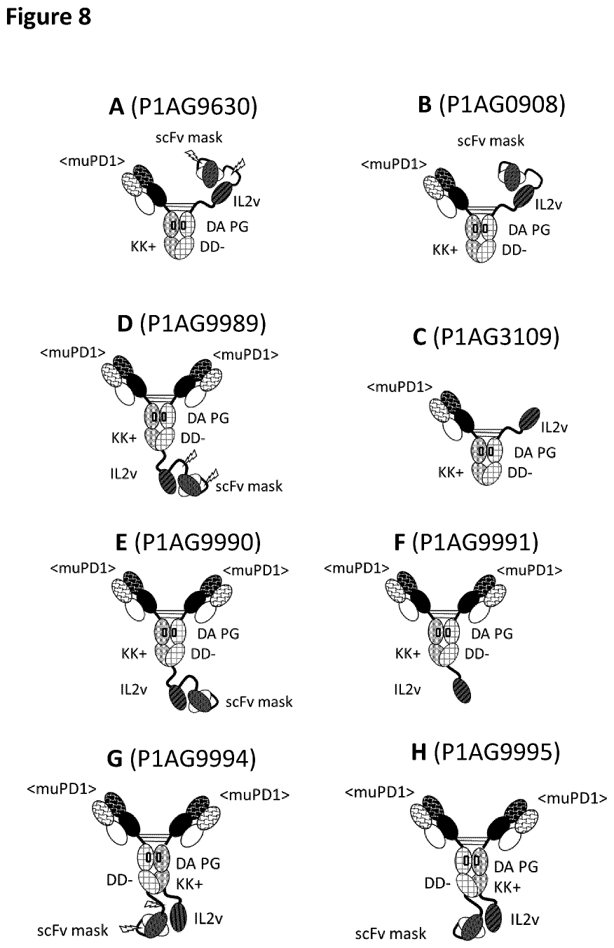

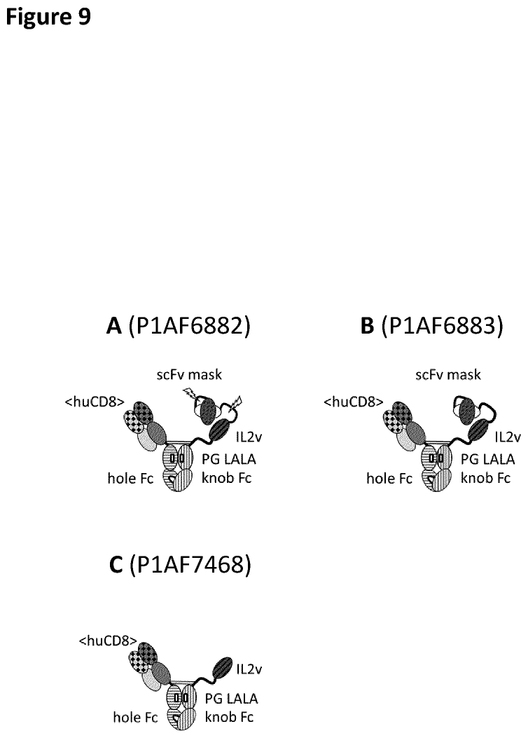

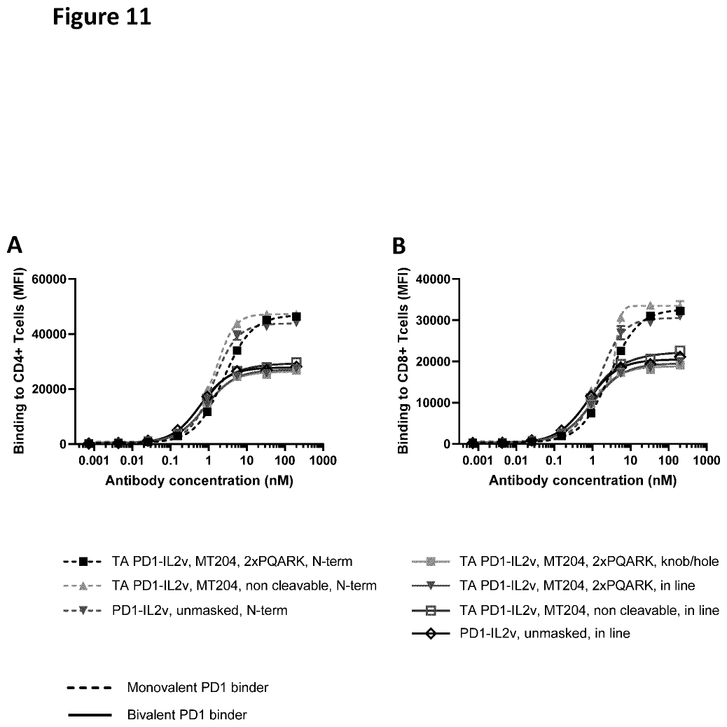

In order to facilitate in vivo tolerability and efficacy studies in non-tumor bearing mice or mouse models of cancer, murine surrogates of PD1-targeted masked IL2v immunoconjugates have been generated. In order to decrease immunogenicity, all constant antibody domains in these constructs correspond to murine sequences. The murine surrogates are either targeted to human PD1 for use in humanized mice or human PD1 transgenic mice (FIGS. 7A-F and FIGS. 10A-E) or murine PD1 for use in syngeneic mouse models with immunocompetent mice (FIGS. 8A-H). Due to the cross-reactivity of human IL2v to murine IL2 receptors and the lack of cross-reactivity of the scFv mask to murine IL2v, human IL2v has been used in all but one control construct (FIG. 10D).

The murine surrogates were generated as one-armed and bivalently human or murine PD1-targeted IL2v immunoconjugates. They bind monovalently or bivalently to human or murine PD1 via the N-terminal Fab fragment(s) on the Fc KK+ chain (one-armed human or murine 0 PD1-targeted constructs) or on the Fc DD- and Fc KK+ chain (bivalently human or murine PD1-targeted constructs) whereas the Fc DD-chain additionally carries a masked (matriptase cleavable or non-matriptase cleavable) or non-masked N- or C-terminal IL2v. The C-terminally masked IL2v constructs either carry IL2v and the mask ‘in-line’ on the same Fc DD-chain or, alternatively, carry IL2v on the Fc KK+ chain and the mask on the Fc DD-chain. Heterodimerization was achieved by application of charge complementarity and binding to activating murine Fcγ receptors has been abrogated by introduction of DA PG mutations in the Fc portion of the antibodies. The matriptase-cleavable N- and C-terminally masked IL2v constructs carry two PQARK or two PMAKK or two YAARKGGI matriptase recognition sites, wherein one PQARK or PMAKK or YAARKGGI matriptase recognition site is positioned in the linker between the VH and VL domains of the scFv mask and another one in the linker between the scFv mask and the IL2v in the ‘in-line’ constructs or between the C-terminus of the Fc hole chain and the scFv mask in the construct with IL2v and mask on two separate heavy chains. Additionally, respective non-matriptase cleavable control constructs (devoid of PQARK or YAARKGGI matriptase recognition sites) and non-masked control constructs (devoid of the scFv mask) have been generated. These constructs are schematically depicted in FIGS. 7A-F, FIGS. 8A-H, and FIGS. 10A-E.

### Example 5B

### Production and Purification of PD1-Targeted Masked IL2v Immunoconjugates and Control Constructs

The human as well as murine surrogate PD1-targeted masked IL2v immunoconjugates and respective controls have been produced and purified by WuXi Biologics. They were transiently expressed in HEK293 and purified in a 2- or 3-column process: 1. MabSelectSuRe LX affinity chromatography (equilibration and 1st wash: 50 mM Tris-HCl, 150 mM NaCl, pH 7.4; 2nd wash: 50 mM Tris-HCl, 150 mM NaCl, pH 7.4, 0.1% Triton 100/114; elution: 100 mM Arg, 140 mM NaCl, pH3.4; stripping: 50 mM NaAc-HAc, pH 3.0; neutralization: 1 M Arg, pH 9.1) with 0.05% Tween20 added for the washing and elution steps if required; 2. if required, HiTrap SP HP cation exchange chromatography (equilibration and 1st wash: 50 mM NaAc-HAc, pH 5.5; elution: 50 mM NaAc-HAc, 2 M NaCl, pH 5.5), and 3. Superdex200 size-exclusion chromatography (equilibration and formulation buffer: 20 mM Histidine-HCl, 140 mM NaCl, pH 6.0). Purity has been determined by SEC-HPLC and reduced and non-reduced Caliper-SDS. Purification batches were tested for low endotoxin levels and the identity of the de-glycosylated masses has been confirmed by liquid chromatography-mass spectrometry (LC-MS).

TABLE 2
Chains and SEQ ID NOs of molecules generated in Examples 5A and 5B

| TAPIR_ID | ID | SEQ | SEQ ID NO |
|---|---|---|---|
| C02011851 | C02011851-A | DIVMTQSPDSLAVSLGERATINCKASESVDTSDNSFIH | 34 |
| | | WYQQKPGQSPKLLIYRSSTLESGVPDRFSGSGSGTDFT | |
| | | LTISSLQAEDVAVYYCQQNYDVPWTFGQGTKVEIKR | |
| | | TVAAPSVFIFPPSDEQLKSGTASVVCLLNNFYPREAKV | |
| | | QWKVDNALQSGNSQESVTEQDSKDSTYSLSSTLTLSK | |
| | | ADYEKHKVYACEVTHQGLSSPVTKSFNRGEC | |
| C2184230856 | C2184230856-H | EVQLLESGGGLVQPGGSLRLSCAASGFSFSSYTMSWV | 35 |
| | | RQAPGKGLEWVATISGGGRDIYYPDSVKGRFTISRDN | |
| | | SKNTLYLQMNSLRAEDTAVYYCVLLTGRVYFALDS | |
| | | WGQGTLVTVSSASTKGPSVFPLAPSSKSTSGGTAALG | |
| | | CLVKDYFPEPVTVSWNSGALTSGVHTFPAVLQSSGLY | |
| | | SLSSVVTVPSSSLGTQTYICNVNHKPSNTKVDKKVEP | |
| | | KSCDKTHTCPPCPAPEAAGGPSVFLFPPKPKDTLMISR | |
| | | TPEVTCVVVDVSHEDPEVKFNWYVDGVEVHNAKTK | |
| | | PREEQYNSTYRVVSVLTVLHQDWLNGKEYKCKVSN | |
| | | KALGAPIEKTISKAKGQPREPQVCTLPPSRDELTKNQV | |
| | | SLSCAVKGFYPSDIAVEWESNGQPENNYKTTPPVLDS | |
| | | DGSFFLVSKLTVDKSRWQQGNVFSCSVMHEALHNHY | |
| | | TQKSLSLSPG | |
| C2184253067 | C2184253067-K | EVQLVESGGGLVQPGGSLRLSCAASGFTFSSYTLAWV | 36 |
| | | RQAPGKGLEWVAAIDSSSYTYSPDTVRGRFTISRDNA | |
| | | KNSLYLQMNSLRAEDTAVYYCARDSNWDALDYWG | |
| | | QGTTVTVSSGGGGSGGPQARKGGGGSGGGDIQMTQS | |
| | | PSSLSASVGDRVTITCKASQNVGTNVGWYQQKPGKA | |
| | | PKALIYSASFRYSGVPSRFSGSGSGTDFTLTISSLQPED | |
| | | FATYYCQQYYTYPYTFGGGTKVEIKGGGGSGGGGSP | |
| | | QARKGGGGSGGGGSGGGGSGGGGSGGGAPASSSTK | |
| | | KTQLQLEHLLLDLQMILNGINNYKNPKLTRMLTAKF | |
| | | AMPKKATELKHLQCLEEELKPLEEVLNGAQSKNFHL | |
| | | RPRDLISNINVIVLELKGSETTFMCEYADETATIVEFLN | |
| | | RWITFAQSIISTLTGGGGSGGGGSDKTHTCPPCPAPEA | |
| | | AGGPSVFLFPPKPKDTLMISRTPEVTCVVVDVSHEDPE | |
| | | VKFNWYVDGVEVHNAKTKPREEQYNSTYRVVSVLT | |
| | | VLHQDWLNGKEYKCKVSNKALGAPIEKTISKAKGQP | |
| | | REPQVYTLPPCRDELTKNQVSLWCLVKGFYPSDIAVE | |
| | | WESNGQPENNYKTTPPVLDSDGSFFLYSKLTVDKSR | |
| | | WQQGNVFSCSVMHEALHNHYTQKSLSLSPG | |
| C01483382 | C01483382-H | EVQLLESGGGLVQPGGSLRLSCAASGFSFSSYTMSWV | 37 |
| | | RQAPGKGLEWVATISGGGRDIYYPDSVKGRFTISRDN | |
| | | SKNTLYLQMNSLRAEDTAVYYCVLLTGRVYFALDS | |
| | | WGQGTLVTVSSASTKGPSVFPLAPSSKSTSGGTAALG | |
| | | CLVKDYFPEPVTVSWNSGALTSGVHTFPAVLQSSGLY | |
| | | SLSSVVTVPSSSLGTQTYICNVNHKPSNTKVDKKVEP | |
| | | KSCDKTHTCPPCPAPEAAGGPSVFLFPPKPKDTLMISR | |
| | | TPEVTCVVVDVSHEDPEVKFNWYVDGVEVHNAKTK | |
| | | PREEQYNSTYRVVSVLTVLHQDWLNGKEYKCKVSN | |
| | | KALGAPIEKTISKAKGQPREPQVCTLPPSRDELTKNQV | |
| | | SLSCAVKGFYPSDIAVEWESNGQPENNYKTTPPVLDS | |
| | | DGSFFLVSKLTVDKSRWQQGNVFSCSVMHEALHNHY | |
| | | TQKSLSLSPGK | |
| C2184220946 | C2184220946-K | EVQLVESGGGLVQPGGSLRLSCAASGFTFSSYTLAWV | 38 |
| | | RQAPGKGLEWVAAIDSSSYTYSPDTVRGRFTISRDNA | |
| | | KNSLYLQMNSLRAEDTAVYYCARDSNWDALDYWG | |
| | | QGTTVTVSSGGGGSGGGGGGSGGGGSGGGDIQMTQS | |
| | | PSSLSASVGDRVTITCKASQNVGTNVGWYQQKPGKA | |
| | | PKALIYSASFRYSGVPSRFSGSGSGTDFTLTISSLQPED | |
| | | FATYYCQQYYTYPYTFGGGTKVEIKGGGGSGGGGSG | |
| | | GGGSGGGGSGGGGSGGGGSGGGGSGGGAPASSSTKK | |
| | | TQLQLEHLLLDLQMILNGINNYKNPKLTRMLTAKFA | |
| | | MPKKATELKHLQCLEEELKPLEEVLNGAQSKNFHLRP | |
| | | RDLISNINVIVLELKGSETTFMCEYADETATIVEFLNR | |
| | | WITFAQSIISTLTGGGGSGGGGSDKTHTCPPCPAPEAA | |
| | | GGPSVFLFPPKPKDTLMISRTPEVTCVVVDVSHEDPEV | |
| | | KFNWYVDGVEVHNAKTKPREEQYNSTYRVVSVLTV | |
| | | LHQDWLNGKEYKCKVSNKALGAPIEKTISKAKGQPR | |
| | | EPQVYTLPPCRDELTKNQVSLWCLVKGFYPSDIAVE | |
| | | WESNGQPENNYKTTPPVLDSDGSFFLYSKLTVDKSR | |
| | | WQQGNVFSCSVMHEALHNHYTQKSLSLSPGK | |
| C2184191702 | C2184191702-K | APASSSTKKTQLQLEHLLLDLQMILNGINNYKNPKLT | 39 |
| | | RMLTAKFAMPKKATELKHLQCLEEELKPLEEVLNGA | |
| | | QSKNFHLRPRDLISNINVIVLELKGSETTFMCEYADET | |
| | | ATIVEFLNRWITFAQSIISTLTGGGGSGGGGSDKTHTC | |
| | | PPCPAPEAAGGPSVFLFPPKPKDTLMISRTPEVTCVVV | |
| | | DVSHEDPEVKFNWYVDGVEVHNAKTKPREEQYNST | |
| | | YRVVSVLTVLHQDWLNGKEYKCKVSNKALGAPIEKT | |
| | | ISKAKGQPREPQVYTLPPCRDELTKNQVSLWCLVKGF | |
| | | YPSDIAVEWESNGQPENNYKTTPPVLDSDGSFFLYSK | |
| | | LTVDKSRWQQGNVFSCSVMHEALHNHYTQKSLSLSP | |
| | | GK | |
| C2184253077 | C2184253077-K | EVQLLESGGGLVQPGGSLRLSCAASGFSFSSYTMSWV | 40 |
| | | RQAPGKGLEWVATISGGGRDIYYPDSVKGRFTISRDN | |
| | | SKNTLYLQMNSLRAEDTAVYYCVLLTGRVYFALDS | |
| | | WGQGTLVTVSSASTKGPSVFPLAPSSKSTSGGTAALG | |
| | | CLVKDYFPEPVTVSWNSGALTSGVHTFPAVLQSSGLY | |
| | | SLSSVVTVPSSSLGTQTYICNVNHKPSNTKVDKKVEP | |
| | | KSCDKTHTCPPCPAPEAAGGPSVFLFPPKPKDTLMISR | |
| | | TPEVTCVVVDVSHEDPEVKFNWYVDGVEVHNAKTK | |
| | | PREEQYNSTYRVVSVLTVLHQDWLNGKEYKCKVSN | |
| | | KALGAPIEKTISKAKGQPREPQVYTLPPCRDELTKNQ | |
| | | VSLWCLVKGFYPSDIAVEWESNGQPENNYKTTPPVL | |
| | | DSDGSFFLYSKLTVDKSRWQQGNVFSCSVMHEALHN | |
| | | HYTQKSLSLSPGGGGGSGGGGSGGGGGAPASSSTKK | |
| | | TQLQLEHLLLDLQMILNGINNYKNPKLTRMLTAKFA | |
| | | MPKKATELKHLQCLEEELKPLEEVLNGAQSKNFHLRP | |
| | | RDLISNINVIVLELKGSETTFMCEYADETATIVEFLNR | |
| | | WITFAQSIISTLTGGGGSGGGGSPQARKGGGGSGGGG | |
| | | SGGGGSGGGGSGGGEVQLVESGGGLVQPGGSLRLSC | |
| | | AASGFTFSSYTLAWVRQAPGKGLEWVAAIDSSSYTYS | |
| | | PDTVRGRFTISRDNAKNSLYLQMNSLRAEDTAVYYC | |
| | | ARDSNWDALDYWGQGTTVTVSSGGGGSGGPQARKG | |
| | | GGGSGGGDIQMTQSPSSLSASVGDRVTITCKASQNVG | |
| | | TNVGWYQQKPGKAPKALIYSASFRYSGVPSRFSGSGS | |
| | | GTDFTLTISSLQPEDFATYYCQQYYTYPYTFGGGTKV | |
| | | EIK | |
| C2184236829 | C2184236829-K | EVQLLESGGGLVQPGGSLRLSCAASGFSFSSYTMSWV | 41 |
| | | RQAPGKGLEWVATISGGGRDIYYPDSVKGRFTISRDN | |
| | | SKNTLYLQMNSLRAEDTAVYYCVLLTGRVYFALDS | |
| | | WGQGTLVTVSSASTKGPSVFPLAPSSKSTSGGTAALG | |
| | | CLVKDYFPEPVTVSWNSGALTSGVHTFPAVLQSSGLY | |
| | | SLSSVVTVPSSSLGTQTYICNVNHKPSNTKVDKKVEP | |
| | | KSCDKTHTCPPCPAPEAAGGPSVFLFPPKPKDTLMISR | |
| | | TPEVTCVVVDVSHEDPEVKFNWYVDGVEVHNAKTK | |
| | | PREEQYNSTYRVVSVLTVLHQDWLNGKEYKCKVSN | |
| | | KALGAPIEKTISKAKGQPREPQVYTLPPCRDELTKNQ | |
| | | VSLWCLVKGFYPSDIAVEWESNGQPENNYKTTPPVL | |
| | | DSDGSFFLYSKLTVDKSRWQQGNVFSCSVMHEALHN | |
| | | HYTQKSLSLSPGGGGGSGGGGSGGGGGAPASSSTKK | |
| | | TQLQLEHLLLDLQMILNGINNYKNPKLTRMLTAKFA | |
| | | MPKKATELKHLQCLEEELKPLEEVLNGAQSKNFHLRP | |
| | | RDLISNINVIVLELKGSETTFMCEYADETATIVEFLNR | |
| | | WITFAQSIISTLTGGGGSGGGGSGGGGSGGGGSGGGG | |
| | | SGGGGSGGGGSGGGGSGEVQLVESGGGLVQPGGSLR | |
| | | LSCAASGFTFSSYTLAWVRQAPGKGLEWVAAIDSSSY | |
| | | TYSPDTVRGRFTISRDNAKNSLYLQMNSLRAEDTAVY | |
| | | YCARDSNWDALDYWGQGTTVTVSSGGGGSGGGGG | |
| | | GSGGGGSGGGDIQMTQSPSSLSASVGDRVTITCKASQ | |
| | | NVGTNVGWYQQKPGKAPKALIYSASFRYSGVPSRFS | |
| | | GSGSGTDFTLTISSLQPEDFATYYCQQYYTYPYTFGG | |
| | | GTKVEIK | |
| C179973954 | C179973954-K | EVQLLESGGGLVQPGGSLRLSCAASGFSFSSYTMSWV | 42 |
| | | RQAPGKGLEWVATISGGGRDIYYPDSVKGRFTISRDN | |
| | | SKNTLYLQMNSLRAEDTAVYYCVLLTGRVYFALDS | |
| | | WGQGTLVTVSSASTKGPSVFPLAPSSKSTSGGTAALG | |
| | | CLVKDYFPEPVTVSWNSGALTSGVHTFPAVLQSSGLY | |
| | | SLSSVVTVPSSSLGTQTYICNVNHKPSNTKVDKKVEP | |
| | | KSCDKTHTCPPCPAPEAAGGPSVFLFPPKPKDTLMISR | |
| | | TPEVTCVVVDVSHEDPEVKFNWYVDGVEVHNAKTK | |
| | | PREEQYNSTYRVVSVLTVLHQDWLNGKEYKCKVSN | |
| | | KALGAPIEKTISKAKGQPREPQVYTLPPCRDELTKNQ | |
| | | VSLWCLVKGFYPSDIAVEWESNGQPENNYKTTPPVL | |
| | | DSDGSFFLYSKLTVDKSRWQQGNVFSCSVMHEALHN | |
| | | HYTQKSLSLSPGGGGGSGGGGSGGGGGAPASSSTKK | |
| | | TQLQLEHLLLDLQMILNGINNYKNPKLTRMLTAKFA | |
| | | MPKKATELKHLQCLEEELKPLEEVLNGAQSKNFHLRP | |
| | | RDLISNINVIVLELKGSETTFMCEYADETATIVEFLNR | |
| | | WITFAQSIISTLT | |
| C2184253078 | C2184253078-H | EVQLLESGGGLVQPGGSLRLSCAASGFSFSSYTMSWV | 43 |
| | | RQAPGKGLEWVATISGGGRDIYYPDSVKGRFTISRDN | |
| | | SKNTLYLQMNSLRAEDTAVYYCVLLTGRVYFALDS | |
| | | WGQGTLVTVSSASTKGPSVFPLAPSSKSTSGGTAALG | |
| | | CLVKDYFPEPVTVSWNSGALTSGVHTFPAVLQSSGLY | |
| | | SLSSVVTVPSSSLGTQTYICNVNHKPSNTKVDKKVEP | |
| | | KSCDKTHTCPPCPAPEAAGGPSVFLFPPKPKDTLMISR | |
| | | TPEVTCVVVDVSHEDPEVKFNWYVDGVEVHNAKTK | |
| | | PREEQYNSTYRVVSVLTVLHQDWLNGKEYKCKVSN | |
| | | KALGAPIEKTISKAKGQPREPQVCTLPPSRDELTKNQV | |
| | | SLSCAVKGFYPSDIAVEWESNGQPENNYKTTPPVLDS | |
| | | DGSFFLVSKLTVDKSRWQQGNVFSCSVMHEALHNHY | |
| | | TQKSLSLSPGGGGGSGGGGSPQARKGGGGSGGGGSG | |
| | | GGGSGGGGSGGGEVQLVESGGGLVQPGGSLRLSCAA | |
| | | SGFTFSSYTLAWVRQAPGKGLEWVAAIDSSSYTYSPD | |
| | | TVRGRFTISRDNAKNSLYLQMNSLRAEDTAVYYCAR | |
| | | DSNWDALDYWGQGTTVTVSSGGGGSGGPQARKGGG | |
| | | GSGGGDIQMTQSPSSLSASVGDRVTITCKASQNVGTN | |
| | | VGWYQQKPGKAPKALIYSASFRYSGVPSRFSGSGSGT | |
| | | DFTLTISSLQPEDFATYYCQQYYTYPYTFGGGTKVEIK | |
| C2184220338 | C2184220338-P | EVILVESGGGLVKPGGSLKLSCAASGFSFSSYTMSWV | 44 |
| | | RQTPEKRLDWVATISGGGRDIYYPDSVKGRFTISRDN | |
| | | AKNTLYLEMSSLMSEDTALYYCVLLTGRVYFALDSW | |
| | | GQGTSVTVSSAKTTPPSVYPLAPGSAAQTNSMVTLGC | |
| | | LVKGYFPEPVTVTWNSGSLSSGVHTFPAVLQSDLYTL | |
| | | SSSVTVPSSTWPSQTVTCNVAHPASSTKVDKKIVPRD | |
| | | CGCKPCICTVPEVSSVFIFPPKPKDVLTITLTPKVTCVV | |
| | | VAISKDDPEVQFSWFVDDVEVHTAQTKPREEQINSTF | |
| | | RSVSELPIMHQDWLNGKEFKCRVNSAAFGAPIEKTIS | |
| | | KTKGRPKAPQVYTIPPPKKQMAKDKVSLTCMITNFFP | |
| | | EDITVEWQWNGQPAENYKNTQPIMKTDGSYFVYSKL | |
| | | NVQKSNWEAGNTFTCSVLHEGLHNHHTEKSLSHSPG | |
| C2184220339 | C2184220339-A | KIVLTQSPASLPVSLGQRATISCRASESVDTSDNSFIH | 45 |
| | | WYQQRPGQSPKLLIYRSSTLESGVPARFSGSGSRTDFT | |
| | | LTIDPVEADDVATYYCQQNYDVPWTFGGGTKLEIKR | |
| | | ADAAPTVSIFPPSSEQLTSGGASVVCFLNNFYPKDINV | |
| | | KWKIDGSERQNGVLNSWTDQDSKDSTYSMSSTLTLT | |
| | | KDEYERHNSYTCEATHKTSTSPIVKSFNRNEC | |
| C2184253107 | C2184253107-M | EVQLVESGGGLVQPGGSLRLSCAASGFTFSSYTLAWV | 46 |
| | | RQAPGKGLEWVAAIDSSSYTYSPDTVRGRFTISRDNA | |
| | | KNSLYLQMNSLRAEDTAVYYCARDSNWDALDYWG | |
| | | QGTTVTVSSGGGGSGGPQARKGGGGSGGGDIQMTQS | |
| | | PSSLSASVGDRVTITCKASQNVGTNVGWYQQKPGKA | |
| | | PKALIYSASFRYSGVPSRFSGSGSGTDFTLTISSLQPED | |
| | | FATYYCQQYYTYPYTFGGGTKVEIKGGGGSGGGGSP | |
| | | QARKGGGGSGGGGSGGGGSGGGGSGGGAPASSSTK | |
| | | KTQLQLEHLLLDLQMILNGINNYKNPKLTRMLTAKF | |
| | | AMPKKATELKHLQCLEEELKPLEEVLNGAQSKNFHL | |
| | | RPRDLISNINVIVLELKGSETTFMCEYADETATIVEFLN | |
| | | RWITFAQSIISTLTGGGGSGGGGSGGGGGCKPCICTVP | |
| | | EVSSVFIFPPKPKDVLTITLTPKVTCVVVAISKDDPEVQ | |
| | | FSWFVDDVEVHTAQTKPREEQINSTFRSVSELPIMHQ | |
| | | DWLNGKEFKCRVNSAAFGAPIEKTISKTKGRPKAPQV | |
| | | YTIPPPKEQMAKDKVSLTCMITNFFPEDITVEWQWNG | |
| | | QPAENYDNTQPIMDTDGSYFVYSDLNVQKSNWEAG | |
| | | NTFTCSVLHEGLHNHHTEKSLSHSPG | |
| C2184228834 | C2184228834-M | EVQLVESGGGLVQPGGSLRLSCAASGFTFSSYTLAWV | 47 |
| | | RQAPGKGLEWVAAIDSSSYTYSPDTVRGRFTISRDNA | |
| | | KNSLYLQMNSLRAEDTAVYYCARDSNWDALDYWG | |
| | | QGTTVTVSSGGGGSGGGGGGSGGGGSGGGDIQMTQS | |
| | | PSSLSASVGDRVTITCKASQNVGTNVGWYQQKPGKA | |
| | | PKALIYSASFRYSGVPSRFSGSGSGTDFTLTISSLQPED | |
| | | FATYYCQQYYTYPYTFGGGTKVEIKGGGGSGGGGSG | |
| | | GGGSGGGGSGGGGSGGGGSGGGGSGGGAPASSSTKK | |
| | | TQLQLEHLLLDLQMILNGINNYKNPKLTRMLTAKFA | |
| | | MPKKATELKHLQCLEEELKPLEEVLNGAQSKNFHLRP | |
| | | RDLISNINVIVLELKGSETTFMCEYADETATIVEFLNR | |
| | | WITFAQSIISTLTGGGGSGGGGSGGGGGCKPCICTVPE | |
| | | VSSVFIFPPKPKDVLTITLTPKVTCVVVAISKDDPEVQF | |
| | | SWFVDDVEVHTAQTKPREEQINSTFRSVSELPIMHQD | |
| | | WLNGKEFKCRVNSAAFGAPIEKTISKTKGRPKAPQVY | |
| | | TIPPPKEQMAKDKVSLTCMITNFFPEDITVEWQWNGQ | |
| | | PAENYDNTQPIMDTDGSYFVYSDLNVQKSNWEAGNT | |
| | | FTCSVLHEGLHNHHTEKSLSHSPG | |
| C2184233647 | C2184233647-M | APASSSTKKTQLQLEHLLLDLQMILNGINNYKNPKLT | 48 |
| | | RMLTAKFAMPKKATELKHLQCLEEELKPLEEVLNGA | |
| | | QSKNFHLRPRDLISNINVIVLELKGSETTFMCEYADET | |
| | | ATIVEFLNRWITFAQSIISTLTGGGGSGGGGSGGGGGC | |
| | | KPCICTVPEVSSVFIFPPKPKDVLTITLTPKVTCVVVAI | |
| | | SKDDPEVQFSWFVDDVEVHTAQTKPREEQINSTFRSV | |
| | | SELPIMHQDWLNGKEFKCRVNSAAFGAPIEKTISKTK | |
| | | GRPKAPQVYTIPPPKEQMAKDKVSLTCMITNFFPEDIT | |
| | | VEWQWNGQPAENYDNTQPIMDTDGSYFVYSDLNVQ | |
| | | KSNWEAGNTFTCSVLHEGLHNHHTEKSLSHSPG | |
| C2184253819 | C2184253819-M | EVILVESGGGLVKPGGSLKLSCAASGFSFSSYTMSWV | 49 |
| | | RQTPEKRLDWVATISGGGRDIYYPDSVKGRFTISRDN | |
| | | AKNTLYLEMSSLMSEDTALYYCVLLTGRVYFALDSW | |
| | | GQGTSVTVSSAKTTPPSVYPLAPGSAAQTNSMVTLGC | |
| | | LVKGYFPEPVTVTWNSGSLSSGVHTFPAVLQSDLYTL | |
| | | SSSVTVPSSTWPSQTVTCNVAHPASSTKVDKKIVPRD | |
| | | CGCKPCICTVPEVSSVFIFPPKPKDVLTITLTPKVTCVV | |
| | | VAISKDDPEVQFSWFVDDVEVHTAQTKPREEQINSTF | |
| | | RSVSELPIMHQDWLNGKEFKCRVNSAAFGAPIEKTIS | |
| | | KTKGRPKAPQVYTIPPPKEQMAKDKVSLTCMITNFFP | |
| | | EDITVEWQWNGQPAENYDNTQPIMDTDGSYFVYSDL | |
| | | NVQKSNWEAGNTFTCSVLHEGLHNHHTEKSLSHSPG | |
| | | GGGGSGGGGSGGGGGAPASSSTKKTQLQLEHLLLDL | |
| | | QMILNGINNYKNPKLTRMLTAKFAMPKKATELKHLQ | |
| | | CLEEELKPLEEVLNGAQSKNFHLRPRDLISNINVIVLE | |
| | | LKGSETTFMCEYADETATIVEFLNRWITFAQSIISTLTG | |
| | | GGGSGGGGSPQARKGGGGSGGGGSGGGGSGGGGSG | |
| | | GGEVQLVESGGGLVQPGGSLRLSCAASGFTFSSYTLA | |
| | | WVRQAPGKGLEWVAAIDSSSYTYSPDTVRGRFTISRD | |
| | | NAKNSLYLQMNSLRAEDTAVYYCARDSNWDALDY | |
| | | WGQGTTVTVSSGGGGSGGPQARKGGGGSGGGDIQM | |
| | | TQSPSSLSASVGDRVTITCKASQNVGTNVGWYQQKP | |
| | | GKAPKALIYSASFRYSGVPSRFSGSGSGTDFTLTISSLQ | |
| | | PEDFATYYCQQYYTYPYTFGGGTKVEIK | |
| C2184253820 | C2184253820-M | EVILVESGGGLVKPGGSLKLSCAASGFSFSSYTMSWV | 50 |
| | | RQTPEKRLDWVATISGGGRDIYYPDSVKGRFTISRDN | |
| | | AKNTLYLEMSSLMSEDTALYYCVLLTGRVYFALDSW | |
| | | GQGTSVTVSSAKTTPPSVYPLAPGSAAQTNSMVTLGC | |
| | | LVKGYFPEPVTVTWNSGSLSSGVHTFPAVLQSDLYTL | |
| | | SSSVTVPSSTWPSQTVTCNVAHPASSTKVDKKIVPRD | |
| | | CGCKPCICTVPEVSSVFIFPPKPKDVLTITLTPKVTCVV | |
| | | VAISKDDPEVQFSWFVDDVEVHTAQTKPREEQINSTF | |
| | | RSVSELPIMHQDWLNGKEFKCRVNSAAFGAPIEKTIS | |
| | | KTKGRPKAPQVYTIPPPKEQMAKDKVSLTCMITNFFP | |
| | | EDITVEWQWNGQPAENYDNTQPIMDTDGSYFVYSDL | |
| | | NVQKSNWEAGNTFTCSVLHEGLHNHHTEKSLSHSPG | |
| | | GGGGSGGGGSGGGGGAPASSSTKKTQLQLEHLLLDL | |
| | | QMILNGINNYKNPKLTRMLTAKFAMPKKATELKHLQ | |
| | | CLEEELKPLEEVLNGAQSKNFHLRPRDLISNINVIVLE | |
| | | LKGSETTFMCEYADETATIVEFLNRWITFAQSIISTLTG | |
| | | GGGSGGGGSGGGGSGGGGSGGGGSGGGGSGGGGSG | |
| | | GGEVQLVESGGGLVQPGGSLRLSCAASGFTFSSYTLA | |
| | | WVRQAPGKGLEWVAAIDSSSYTYSPDTVRGRFTISRD | |
| | | NAKNSLYLQMNSLRAEDTAVYYCARDSNWDALDY | |
| | | WGQGTTVTVSSGGGGSGGGGGGSGGGGSGGGDIQM | |
| | | TQSPSSLSASVGDRVTITCKASQNVGTNVGWYQQKP | |
| | | GKAPKALIYSASFRYSGVPSRFSGSGSGTDFTLTISSLQ | |
| | | PEDFATYYCQQYYTYPYTFGGGTKVEIK | |
| C2184248915 | C2184248915-M | EVILVESGGGLVKPGGSLKLSCAASGFSFSSYTMSWV | 51 |
| | | RQTPEKRLDWVATISGGGRDIYYPDSVKGRFTISRDN | |
| | | AKNTLYLEMSSLMSEDTALYYCVLLTGRVYFALDSW | |
| | | GQGTSVTVSSAKTTPPSVYPLAPGSAAQTNSMVTLGC | |
| | | LVKGYFPEPVTVTWNSGSLSSGVHTFPAVLQSDLYTL | |
| | | SSSVTVPSSTWPSQTVTCNVAHPASSTKVDKKIVPRD | |
| | | CGCKPCICTVPEVSSVFIFPPKPKDVLTITLTPKVTCVV | |
| | | VAISKDDPEVQFSWFVDDVEVHTAQTKPREEQINSTF | |
| | | RSVSELPIMHQDWLNGKEFKCRVNSAAFGAPIEKTIS | |
| | | KTKGRPKAPQVYTIPPPKEQMAKDKVSLTCMITNFFP | |
| | | EDITVEWQWNGQPAENYDNTQPIMDTDGSYFVYSDL | |
| | | NVQKSNWEAGNTFTCSVLHEGLHNHHTEKSLSHSPG | |
| | | GGGGSGGGGSGGGGGAPASSSTKKTQLQLEHLLLDL | |
| | | QMILNGINNYKNPKLTRMLTAKFAMPKKATELKHLQ | |
| | | CLEEELKPLEEVLNGAQSKNFHLRPRDLISNINVIVLE | |
| | | LKGSETTFMCEYADETATIVEFLNRWITFAQSIISTLT | |
| C01486562 | C01486562-A | DIVMTQGTLPNPVPSGESVSITCRSSKSLLYSDGKTYL | 52 |
| | | NWYLQRPGQSPQLLIYWMSTRASGVSDRFSGSGSGT | |
| | | DFTLKISGVEAEDVGIYYCQQGLEFPTFGGGTKLELK | |
| | | RADAAPTVSIFPPSSEQLTSGGASVVCFLNNFYPKDIN | |
| | | VKWKIDGSERQNGVLNSWTDQDSKDSTYSMSSTLTL | |
| | | TKDEYERHNSYTCEATHKTSTSPIVKSFNRNEC | |
| C2184220341 | C2184220341-P | EVQLQESGPGLVKPSQSLSLTCSVTGYSITSSYRWNWI | 53 |
| | | RKFPGNRLEWMGYINSAGISNYNPSLKRRISITRDTSK | |
| | | NQFFLQVNSVTTEDAATYYCARSDNMGTTPFTYWGQ | |
| | | GTLVTVSSAKTTPPSVYPLAPGSAAQTNSMVTLGCLV | |
| | | KGYFPEPVTVTWNSGSLSSGVHTFPAVLQSDLYTLSS | |
| | | SVTVPSSTWPSQTVTCNVAHPASSTKVDKKIVPRDCG | |
| | | CKPCICTVPEVSSVFIFPPKPKDVLTITLTPKVTCVVVA | |
| | | ISKDDPEVQFSWFVDDVEVHTAQTKPREEQINSTFRS | |
| | | VSELPIMHQDWLNGKEFKCRVNSAAFGAPIEKTISKT | |
| | | KGRPKAPQVYTIPPPKKQMAKDKVSLTCMITNFFPED | |
| | | ITVEWQWNGQPAENYKNTQPIMKTDGSYFVYSKLNV | |
| | | QKSNWEAGNTFTCSVLHEGLHNHHTEKSLSHSPG | |
| C2184253824 | C2184253824-M | EVQLQESGPGLVKPSQSLSLTCSVTGYSITSSYRWNWI | 54 |
| | | RKFPGNRLEWMGYINSAGISNYNPSLKRRISITRDTSK | |
| | | NQFFLQVNSVTTEDAATYYCARSDNMGTTPFTYWGQ | |
| | | GTLVTVSSAKTTPPSVYPLAPGSAAQTNSMVTLGCLV | |
| | | KGYFPEPVTVTWNSGSLSSGVHTFPAVLQSDLYTLSS | |
| | | SVTVPSSTWPSQTVTCNVAHPASSTKVDKKIVPRDCG | |
| | | CKPCICTVPEVSSVFIFPPKPKDVLTITLTPKVTCVVVA | |
| | | ISKDDPEVQFSWFVDDVEVHTAQTKPREEQINSTFRS | |
| | | VSELPIMHQDWLNGKEFKCRVNSAAFGAPIEKTISKT | |
| | | KGRPKAPQVYTIPPPKEQMAKDKVSLTCMITNFFPEDI | |
| | | TVEWQWNGQPAENYDNTQPIMDTDGSYFVYSDLNV | |
| | | QKSNWEAGNTFTCSVLHEGLHNHHTEKSLSHSPGGG | |
| | | GGSGGGGSGGGGGAPASSSTKKTQLQLEHLLLDLQM | |
| | | ILNGINNYKNPKLTRMLTAKFAMPKKATELKHLQCL | |
| | | EEELKPLEEVLNGAQSKNFHLRPRDLISNINVIVLELK | |
| | | GSETTFMCEYADETATIVEFLNRWITFAQSIISTLTGG | |
| | | GGSGGGGSPQARKGGGGSGGGGSGGGGSGGGGSGG | |
| | | GEVQLVESGGGLVQPGGSLRLSCAASGFTFSSYTLAW | |
| | | VRQAPGKGLEWVAAIDSSSYTYSPDTVRGRFTISRDN | |
| | | AKNSLYLQMNSLRAEDTAVYYCARDSNWDALDYW | |
| | | GQGTTVTVSSGGGGSGGPQARKGGGGSGGGDIQMT | |
| | | QSPSSLSASVGDRVTITCKASQNVGTNVGWYQQKPG | |
| | | KAPKALIYSASFRYSGVPSRFSGSGSGTDFTLTISSLQP | |
| | | EDFATYYCQQYYTYPYTFGGGTKVEIK | |
| C2184253825 | C2184253825-M | EVQLQESGPGLVKPSQSLSLTCSVTGYSITSSYRWNWI | 55 |
| | | RKFPGNRLEWMGYINSAGISNYNPSLKRRISITRDTSK | |
| | | NQFFLQVNSVTTEDAATYYCARSDNMGTTPFTYWGQ | |
| | | GTLVTVSSAKTTPPSVYPLAPGSAAQTNSMVTLGCLV | |
| | | KGYFPEPVTVTWNSGSLSSGVHTFPAVLQSDLYTLSS | |
| | | SVTVPSSTWPSQTVTCNVAHPASSTKVDKKIVPRDCG | |
| | | CKPCICTVPEVSSVFIFPPKPKDVLTITLTPKVTCVVVA | |
| | | ISKDDPEVQFSWFVDDVEVHTAQTKPREEQINSTFRS | |
| | | VSELPIMHQDWLNGKEFKCRVNSAAFGAPIEKTISKT | |
| | | KGRPKAPQVYTIPPPKEQMAKDKVSLTCMITNFFPEDI | |
| | | TVEWQWNGQPAENYDNTQPIMDTDGSYFVYSDLNV | |
| | | QKSNWEAGNTFTCSVLHEGLHNHHTEKSLSHSPGGG | |
| | | GGSGGGGSGGGGGAPASSSTKKTQLQLEHLLLDLQM | |
| | | ILNGINNYKNPKLTRMLTAKFAMPKKATELKHLQCL | |
| | | EEELKPLEEVLNGAQSKNFHLRPRDLISNINVIVLELK | |
| | | GSETTFMCEYADETATIVEFLNRWITFAQSIISTLTGG | |
| | | GGSGGGGSGGGGSGGGGSGGGGSGGGGSGGGGSGG | |
| | | GEVQLVESGGGLVQPGGSLRLSCAASGFTFSSYTLAW | |
| | | VRQAPGKGLEWVAAIDSSSYTYSPDTVRGRFTISRDN | |
| | | AKNSLYLQMNSLRAEDTAVYYCARDSNWDALDYW | |
| | | GQGTTVTVSSGGGGSGGGGGGSGGGGSGGGDIQMT | |
| | | QSPSSLSASVGDRVTITCKASQNVGTNVGWYQQKPG | |
| | | KAPKALIYSASFRYSGVPSRFSGSGSGTDFTLTISSLQP | |
| | | EDFATYYCQQYYTYPYTFGGGTKVEIK | |
| C2184253826 | C2184253826-M | EVQLQESGPGLVKPSQSLSLTCSVTGYSITSSYRWNWI | 56 |
| | | RKFPGNRLEWMGYINSAGISNYNPSLKRRISITRDTSK | |
| | | NQFFLQVNSVTTEDAATYYCARSDNMGTTPFTYWGQ | |
| | | GTLVTVSSAKTTPPSVYPLAPGSAAQTNSMVTLGCLV | |
| | | KGYFPEPVTVTWNSGSLSSGVHTFPAVLQSDLYTLSS | |
| | | SVTVPSSTWPSQTVTCNVAHPASSTKVDKKIVPRDCG | |
| | | CKPCICTVPEVSSVFIFPPKPKDVLTITLTPKVTCVVVA | |
| | | ISKDDPEVQFSWFVDDVEVHTAQTKPREEQINSTFRS | |
| | | VSELPIMHQDWLNGKEFKCRVNSAAFGAPIEKTISKT | |
| | | KGRPKAPQVYTIPPPKEQMAKDKVSLTCMITNFFPEDI | |
| | | TVEWQWNGQPAENYDNTQPIMDTDGSYFVYSDLNV | |
| | | QKSNWEAGNTFTCSVLHEGLHNHHTEKSLSHSPGGG | |
| | | GGSGGGGSGGGGGAPASSSTKKTQLQLEHLLLDLQM | |
| | | ILNGINNYKNPKLTRMLTAKFAMPKKATELKHLQCL | |
| | | EEELKPLEEVLNGAQSKNFHLRPRDLISNINVIVLELK | |
| | | GSETTFMCEYADETATIVEFLNRWITFAQSIISTLT | |
| C2184253830 | C2184253830-M | EVQLQESGPGLVKPSQSLSLTCSVTGYSITSSYRWNWI | 57 |
| | | RKFPGNRLEWMGYINSAGISNYNPSLKRRISITRDTSK | |
| | | NQFFLQVNSVTTEDAATYYCARSDNMGTTPFTYWGQ | |
| | | GTLVTVSSAKTTPPSVYPLAPGSAAQTNSMVTLGCLV | |
| | | KGYFPEPVTVTWNSGSLSSGVHTFPAVLQSDLYTLSS | |
| | | SVTVPSSTWPSQTVTCNVAHPASSTKVDKKIVPRDCG | |
| | | CKPCICTVPEVSSVFIFPPKPKDVLTITLTPKVTCVVVA | |
| | | ISKDDPEVQFSWFVDDVEVHTAQTKPREEQINSTFRS | |
| | | VSELPIMHQDWLNGKEFKCRVNSAAFGAPIEKTISKT | |
| | | KGRPKAPQVYTIPPPKEQMAKDKVSLTCMITNFFPEDI | |
| | | TVEWQWNGQPAENYDNTQPIMDTDGSYFVYSDLNV | |
| | | QKSNWEAGNTFTCSVLHEGLHNHHTEKSLSHSPGGG | |
| | | GGSGGGGSPQARKGGGGSGGGGSGGGGSGGGGSGG | |
| | | GEVQLVESGGGLVQPGGSLRLSCAASGFTFSSYTLAW | |
| | | VRQAPGKGLEWVAAIDSSSYTYSPDTVRGRFTISRDN | |
| | | AKNSLYLQMNSLRAEDTAVYYCARDSNWDALDYW | |
| | | GQGTTVTVSSGGGGSGGPQARKGGGGSGGGDIQMT | |
| | | QSPSSLSASVGDRVTITCKASQNVGTNVGWYQQKPG | |
| | | KAPKALIYSASFRYSGVPSRFSGSGSGTDFTLTISSLQP | |
| | | EDFATYYCQQYYTYPYTFGGGTKVEIK | |
| C2184253831 | C2184253831-P | EVQLQESGPGLVKPSQSLSLTCSVTGYSITSSYRWNWI | 58 |
| | | RKFPGNRLEWMGYINSAGISNYNPSLKRRISITRDTSK | |
| | | NQFFLQVNSVTTEDAATYYCARSDNMGTTPFTYWGQ | |
| | | GTLVTVSSAKTTPPSVYPLAPGSAAQTNSMVTLGCLV | |
| | | KGYFPEPVTVTWNSGSLSSGVHTFPAVLQSDLYTLSS | |
| | | SVTVPSSTWPSQTVTCNVAHPASSTKVDKKIVPRDCG | |
| | | CKPCICTVPEVSSVFIFPPKPKDVLTITLTPKVTCVVVA | |
| | | ISKDDPEVQFSWFVDDVEVHTAQTKPREEQINSTFRS | |
| | | VSELPIMHQDWLNGKEFKCRVNSAAFGAPIEKTISKT | |
| | | KGRPKAPQVYTIPPPKKQMAKDKVSLTCMITNFFPED | |
| | | ITVEWQWNGQPAENYKNTQPIMKTDGSYFVYSKLNV | |
| | | QKSNWEAGNTFTCSVLHEGLHNHHTEKSLSHSPGGG | |
| | | GGSGGGGSGGGGGAPASSSTKKTQLQLEHLLLDLQM | |
| | | ILNGINNYKNPKLTRMLTAKFAMPKKATELKHLQCL | |
| | | EEELKPLEEVLNGAQSKNFHLRPRDLISNINVIVLELK | |
| | | GSETTFMCEYADETATIVEFLNRWITFAQSIISTLT | |
| C2184253832 | C2184253832-M | EVQLQESGPGLVKPSQSLSLTCSVTGYSITSSYRWNWI | 59 |
| | | RKFPGNRLEWMGYINSAGISNYNPSLKRRISITRDTSK | |
| | | NQFFLQVNSVTTEDAATYYCARSDNMGTTPFTYWGQ | |
| | | GTLVTVSSAKTTPPSVYPLAPGSAAQTNSMVTLGCLV | |
| | | KGYFPEPVTVTWNSGSLSSGVHTFPAVLQSDLYTLSS | |
| | | SVTVPSSTWPSQTVTCNVAHPASSTKVDKKIVPRDCG | |
| | | CKPCICTVPEVSSVFIFPPKPKDVLTITLTPKVTCVVVA | |
| | | ISKDDPEVQFSWFVDDVEVHTAQTKPREEQINSTFRS | |
| | | VSELPIMHQDWLNGKEFKCRVNSAAFGAPIEKTISKT | |
| | | KGRPKAPQVYTIPPPKEQMAKDKVSLTCMITNFFPEDI | |
| | | TVEWQWNGQPAENYDNTQPIMDTDGSYFVYSDLNV | |
| | | QKSNWEAGNTFTCSVLHEGLHNHHTEKSLSHSPGGG | |
| | | GGSGGGGSGGGGSGGGGSGGGGSGGGGSGGGGSGG | |
| | | GEVQLVESGGGLVQPGGSLRLSCAASGFTFSSYTLAW | |
| | | VRQAPGKGLEWVAAIDSSSYTYSPDTVRGRFTISRDN | |
| | | AKNSLYLQMNSLRAEDTAVYYCARDSNWDALDYW | |
| | | GQGTTVTVSSGGGGSGGGGGGSGGGGSGGGDIQMT | |
| | | QSPSSLSASVGDRVTITCKASQNVGTNVGWYQQKPG | |
| | | KAPKALIYSASFRYSGVPSRFSGSGSGTDFTLTISSLQP | |
| | | EDFATYYCQQYYTYPYTFGGGTKVEIK | |
| C121344880 | C121344880-H | EVQLVQSGAEVKKPGASVKVSCKASGFNIKDTYIHW | 60 |
| | | VRQAPGQGLEWIGRIDPANDNTLYASKFQGRATITAD | |
| | | TSTSTAYLELSSLRSEDTAVYYCGRGYGYYVFDHWG | |
| | | QGTLVTVSSASTKGPSVFPLAPSSKSTSGGTAALGCLV | |
| | | KDYFPEPVTVSWNSGALTSGVHTFPAVLQSSGLYSLS | |
| | | SVVTVPSSSLGTQTYICNVNHKPSNTKVDKKVEPKSC | |
| | | DKTHTCPPCPAPEAAGGPSVFLFPPKPKDTLMISRTPE | |
| | | VTCVVVDVSHEDPEVKFNWYVDGVEVHNAKTKPRE | |
| | | EQYNSTYRVVSVLTVLHQDWLNGKEYKCKVSNKAL | |
| | | GAPIEKTISKAKGQPREPQVCTLPPSRDELTKNQVSLS | |
| | | CAVKGFYPSDIAVEWESNGQPENNYKTTPPVLDSDGS | |
| | | FFLVSKLTVDKSRWQQGNVFSCSVMHEALHNHYTQ | |
| | | KSLSLSPGK | |
| C2184200901 | C2184200901-A | DVQITQSPSSLSASVGDRVTITCRTSRSISQYLAWYQE | 61 |
| | | KPGKTNKLLIYSGSTLQSGIPSRFSGSGSGTDFTLTISSL | |
| | | QPEDFATYYCQQVNEFPPTFGQGTKVEIKRTVAAPSV | |
| | | FIFPPSDEQLKSGTASVVCLLNNFYPREAKVQWKVDN | |
| | | ALQSGNSQESVTEQDSKDSTYSLSSTLTLSKADYEKH | |
| | | KVYACEVTHQGLSSPVTKSFNRGEC | |
| C2184220944 | C2184220944-K | EVQLVESGGGLVQPGGSLRLSCAASGFTFSSYTLAWV | 62 |
| | | RQAPGKGLEWVAAIDSSSYTYSPDTVRGRFTISRDNA | |
| | | KNSLYLQMNSLRAEDTAVYYCARDSNWDALDYWG | |
| | | QGTTVTVSSGGGGSGGPMAKKGGGGSGGGDIQMTQ | |
| | | SPSSLSASVGDRVTITCKASQNVGTNVGWYQQKPGK | |
| | | APKALIYSASFRYSGVPSRFSGSGSGTDFTLTISSLQPE | |
| | | DFATYYCQQYYTYPYTFGGGTKVEIKGGGGSGGGGS | |
| | | PMAKKGGGGSGGGGSGGGGSGGGGSGGGAPASSST | |
| | | KKTQLQLEHLLLDLQMILNGINNYKNPKLTRMLTAK | |
| | | FAMPKKATELKHLQCLEEELKPLEEVLNGAQSKNFH | |
| | | LRPRDLISNINVIVLELKGSETTFMCEYADETATIVEFL | |
| | | NRWITFAQSIISTLTGGGGSGGGGSDKTHTCPPCPAPE | |
| | | AAGGPSVFLFPPKPKDTLMISRTPEVTCVVVDVSHED | |
| | | PEVKFNWYVDGVEVHNAKTKPREEQYNSTYRVVSV | |
| | | LTVLHQDWLNGKEYKCKVSNKALGAPIEKTISKAKG | |
| | | QPREPQVYTLPPCRDELTKNQVSLWCLVKGFYPSDIA | |
| | | VEWESNGQPENNYKTTPPVLDSDGSFFLYSKLTVDKS | |
| | | RWQQGNVFSCSVMHEALHNHYTQKSLSLSPGK | |
| C2184228838 | C2184228838-M | EVQLVESGGGLVQPGGSLRLSCAASGFTFSSYTLAWV | 63 |
| | | RQAPGKGLEWVAAIDSSSYTYSPDTVRGRFTISRDNA | |
| | | KNSLYLQMNSLRAEDTAVYYCARDSNWDALDYWG | |
| | | QGTTVTVSSGGGGSGGPMAKKGGGGSGGGDIQMTQ | |
| | | SPSSLSASVGDRVTITCKASQNVGTNVGWYQQKPGK | |
| | | APKALIYSASFRYSGVPSRFSGSGSGTDFTLTISSLQPE | |
| | | DFATYYCQQYYTYPYTFGGGTKVEIKGGGGSGGGGS | |
| | | PMAKKGGGGSGGGGSGGGGSGGGGSGGGAPASSST | |
| | | KKTQLQLEHLLLDLQMILNGINNYKNPKLTRMLTAK | |
| | | FAMPKKATELKHLQCLEEELKPLEEVLNGAQSKNFH | |
| | | LRPRDLISNINVIVLELKGSETTFMCEYADETATIVEFL | |
| | | NRWITFAQSIISTLTGGGGSGGGGSGGGGGCKPCICTV | |
| | | PEVSSVFIFPPKPKDVLTITLTPKVTCVVVAISKDDPEV | |
| | | QFSWFVDDVEVHTAQTKPREEQINSTFRSVSELPIMH | |
| | | QDWLNGKEFKCRVNSAAFGAPIEKTISKTKGRPKAPQ | |
| | | VYTIPPPKEQMAKDKVSLTCMITNFFPEDITVEWQWN | |
| | | GQPAENYDNTQPIMDTDGSYFVYSDLNVQKSNWEA | |
| | | GNTFTCSVLHEGLHNHHTEKSLSHSPG | |
| C147684951 | C147684951-A | KIVLTQSPASLPVSLGQRATISCRASESVDTSDNSFIH | 64 |
| | | WYQQRPGQSPKLLIYRSSTLESGVPARFSGSGSRTDFT | |
| | | LTIDPVEADDVATYYCQQNYDVPWTFGGGTKLEIKR | |
| | | TDAAPTVSIFPPSSEQLTSGGASVVCFLNNFYPKDINV | |
| | | KWKIDGSERQNGVLNSWTDQDSKDSTYSMSSTLTLT | |
| | | KDEYERHNSYTCEATHKTSTSPIVKSFNRNEC | |
| C147684969 | C147684969-M | EVILVESGGGLVKPGGSLKLSCAASGFSFSSYTMSWV | 65 |
| | | RQTPEKRLDWVATISGGGRDIYYPDSVKGRFTISRDN | |
| | | AKNTLYLEMSSLMSEDTALYYCVLLTGRVYFALDSW | |
| | | GQGTSVTVSSAKTTPPSVYPLAPGSAAQTNSMVTLGC | |
| | | LVKGYFPEPVTVTWNSGSLSSGVHTFPAVLQSDLYTL | |
| | | SSSVTVPSSTWPSQTVTCNVAHPASSTKVDKKIVPRD | |
| | | CGCKPCICTVPEVSSVFIFPPKPKDVLTITLTPKVTCVV | |
| | | VAISKDDPEVQFSWFVDDVEVHTAQTKPREEQINSTF | |
| | | RSVSELPIMHQDWLNGKEFKCRVNSAAFGAPIEKTIS | |
| | | KTKGRPKAPQVYTIPPPKEQMAKDKVSLTCMITNFFP | |
| | | EDITVEWQWNGQPAENYDNTQPIMDTDGSYFVYSDL | |
| | | NVQKSNWEAGNTFTCSVLHEGLHNHHTEKSLSHSPG | |
| | | GGGGSGGGGSGGGGSAPASSSTSSSTAEAQQQQQQQ | |
| | | QQQQQHLEQLLMDLQELLSRMENYRNLKLPRMLTA | |
| | | KFALPKQATELKDLQCLEDELGPLRHVLDGTQSKSFQ | |
| | | LEDAENFISNIRVTVVKLKGSDNTFECQFDDESATVV | |
| | | DFLRRWIAFAQSIISTSPQ | |
| C147685020 | C147685020-P | EVILVESGGGLVKPGGSLKLSCAASGFSFSSYTMSWV | 66 |
| | | RQTPEKRLDWVATISGGGRDIYYPDSVKGRFTISRDN | |
| | | AKNTLYLEMSSLMSEDTALYYCVLLTGRVYFALDSW | |
| | | GQGTSVTVSSAKTTPPSVYPLAPGSAAQTNSMVTLGC | |
| | | LVKGYFPEPVTVTWNSGSLSSGVHTFPAVLQSDLYTL | |
| | | SSSVTVPSSTWPSQTVTCNVAHPASSTKVDKKIVPRD | |
| | | CGCKPCICTVPEVSSVFIFPPKPKDVLTITLTPKVTCVV | |
| | | VAISKDDPEVQFSWFVDDVEVHTAQTKPREEQINSTF | |
| | | RSVSELPIMHQDWLNGKEFKCRVNSAAFGAPIEKTIS | |
| | | KTKGRPKAPQVYTIPPPKKQMAKDKVSLTCMITNFFP | |
| | | EDITVEWQWNGQPAENYKNTQPIMKTDGSYFVYSKL | |
| | | NVQKSNWEAGNTFTCSVLHEGLHNHHTEKSLSHSPG | |
| | | K | |
| C2184229955 | C2184229955-M | EVQLVESGGGLVQPGGSLRLSCAASGFTFSSYTLAWV | 67 |
| | | RQAPGKGLEWVAAIDSSSYTYSPDTVRGRFTISRDNA | |
| | | KNSLYLQMNSLRAEDTAVYYCARDSNWDALDYWG | |
| | | QGTTVTVSSGGGGSGGYAARKGGIGGGGSGGGDIQM | |
| | | TQSPSSLSASVGDRVTITCKASQNVGTNVGWYQQKP | |
| | | GKAPKALIYSASFRYSGVPSRFSGSGSGTDFTLTISSLQ | |
| | | PEDFATYYCQQYYTYPYTFGGGTKVEIKGGGGSGGG | |
| | | GSYAARKGGIGGGGSGGGGSGGGGSGGGGSGGGAP | |
| | | ASSSTKKTQLQLEHLLLDLQMILNGINNYKNPKLTRM | |
| | | LTAKFAMPKKATELKHLQCLEEELKPLEEVLNGAQS | |
| | | KNFHLRPRDLISNINVIVLELKGSETTFMCEYADETAT | |
| | | IVEFLNRWITFAQSIISTLTGGGGSGGGGSGGGGGCKP | |
| | | CICTVPEVSSVFIFPPKPKDVLTITLTPKVTCVVVAISK | |
| | | DDPEVQFSWFVDDVEVHTAQTKPREEQINSTFRSVSE | |
| | | LPIMHQDWLNGKEFKCRVNSAAFGAPIEKTISKTKGR | |
| | | PKAPQVYTIPPPKEQMAKDKVSLTCMITNFFPEDITVE | |
| | | WQWNGQPAENYDNTQPIMDTDGSYFVYSDLNVQKS | |
| | | NWEAGNTFTCSVLHEGLHNHHTEKSLSHSPG | |
| C01478648 | C01478648-H | EVQLVESGGGLVKPGGSLRLSCAASGFTFSNAWMSW | 68 |
| | | VRQAPGKGLEWVGRIKSKTDGGTTDYAAPVKGRFTI | |
| | | SRDDSKNTLYLQMNSLKTEDTAVYYCTTPWEWSWY | |
| | | DYWGQGTLVTVSSASTKGPSVFPLAPSSKSTSGGTAA | |
| | | LGCLVKDYFPEPVTVSWNSGALTSGVHTFPAVLQSSG | |
| | | LYSLSSVVTVPSSSLGTQTYICNVNHKPSNTKVDKKV | |
| | | EPKSCDKTHTCPPCPAPEAAGGPSVFLFPPKPKDTLMI | |
| | | SRTPEVTCVVVDVSHEDPEVKFNWYVDGVEVHNAK | |
| | | TKPREEQYNSTYRVVSVLTVLHQDWLNGKEYKCKVS | |
| | | NKALGAPIEKTISKAKGQPREPQVCTLPPSRDELTKNQ | |
| | | VSLSCAVKGFYPSDIAVEWESNGQPENNYKTTPPVLD | |
| | | SDGSFFLVSKLTVDKSRWQQGNVFSCSVMHEALHNR | |
| | | FTQKSLSLSPGK | |
| C08611149 | C08611149-K | EVQLLESGGGLVQPGGSLRLSCAASGFTFSTYAMNW | 69 |
| | | VRQAPGKGLEWVSRIRSKYNNYATYYADSVKGRFTI | |
| | | SRDDSKNTLYLQMNSLRAEDTAVYYCVRHGNFGNS | |
| | | YVSWFAYWGQGTLVTVSSASTKGPSVFPLAPSSKSTS | |
| | | GGTAALGCLVKDYFPEPVTVSWNSGALTSGVHTFPA | |
| | | VLQSSGLYSLSSVVTVPSSSLGTQTYICNVNHKPSNTK | |
| | | VDKKVEPKSCDGGGGSGGGGSEVQLVESGGGLVKPG | |
| | | GSLRLSCAASGFTFSNAWMSWVRQAPGKGLEWVGRI | |
| | | KSKTDGGTTDYAAPVKGRFTISRDDSKNTLYLQMNS | |
| | | LKTEDTAVYYCTTPWEWSWYDYWGQGTLVTVSSAS | |
| | | TKGPSVFPLAPSSKSTSGGTAALGCLVKDYFPEPVTVS | |
| | | WNSGALTSGVHTFPAVLQSSGLYSLSSVVTVPSSSLG | |
| | | TQTYICNVNHKPSNTKVDKKVEPKSCDKTHTCPPCPA | |
| | | PEAAGGPSVFLFPPKPKDTLMISRTPEVTCVVVDVSHE | |
| | | DPEVKFNWYVDGVEVHNAKTKPREEQYNSTYRVVS | |
| | | VLTVLHQDWLNGKEYKCKVSNKALGAPIEKTISKAK | |
| | | GQPREPQVYTLPPCRDELTKNQVSLWCLVKGFYPSDI | |
| | | AVEWESNGQPENNYKTTPPVLDSDGSFFLYSKLTVD | |
| | | KSRWQQGNVFSCSVMHEALHNHYTQKSLSLSPGK | |
| C08611380 | C08611380-A | QAVVTQEPSLTVSPGGTVTLTCGSSTGAVTTSNYAN | 70 |
| | | WVQEKPGQAFRGLIGGTNKRAPGTPARFSGSLLGGK | |
| | | AALTLSGAQPEDEAEYYCALWYSNLWVFGGGTKLT | |
| | | VLGQPKAAPSVTLFPPSSEELQANKATLVCLISDFYPG | |
| | | AVTVAWKADSSPVKAGVETTTPSKQSNNKYAASSYL | |
| | | SLTPEQWKSHRSYSCQVTHEGSTVEKTVAPTECS | |

TABLE 3
Chain composition of molecules generated in Examples 5A and 5B

| MOLECULE | CHAIN 1 | CHAIN 2 | CHAIN 3 |
|---|---|---|---|
| P1AG9597 | C02011851-A | C2184230856-H | C2184253067-K |
| P1AG0929 | C02011851-A | C01483382-H | C2184220946-K |
| P1AG3071 | C02011851-A | C01483382-H | C2184191702-K |
| P1AG9606 | C02011851-A (2x) | C2184230856-H | C2184253077-K |
| P1AG5740 | C02011851-A (2x) | C2184230856-H | C2184236829-K |
| P1AG5741 = P1AA7146 | C02011851-A (2x) | C2184230856-H | C179973954-K |
| P1AG9607 | C02011851-A (2x) | C2184253078-H | C179973954-K |
| P1AG9629 | C2184220339-A | C2184220338-P | C2184253107-M |
| P1AG0905 | C2184220339-A | C2184220338-P | C2184228834-M |
| P1AG3108 | C2184220339-A | C2184220338-P | C2184233647-M |
| P1AG9983 | C2184220339-A (2x) | C2184220338-P | C2184253819-M |
| P1AG9984 | C2184220339-A (2x) | C2184220338-P | C2184253820-M |
| P1AG7552 | C2184220339-A (2x) | C2184220338-P | C2184248915-M |
| P1AG9630 | C01486562-A | C2184220341-P | C2184253107-M |
| P1AG0908 | C01486562-A | C2184220341-P | C2184220341-M |
| P1AG3109 | C01486562-A | C2184220341-P | C2184233647-M |
| P1AG9989 | C01486562-A (2x) | C2184220341-P | C2184253824-M |
| P1AG9990 | C01486562-A (2x) | C2184220341-P | C2184253825-M |
| P1AG9991 | C01486562-A (2x) | C2184220341-P | C2184253826-M |
| P1AG9994 | C01486562-A (2x) | C2184253831-P | C2184253830-M |
| P1AG9995 | C01486562-A (2x) | C2184253831-P | C2184253832-M |
| P1AF6882 | C2184200901-A | C121344880-H | C2184220944-K |
| P1AF6883 | C2184200901-A | C121344880-H | C2184220946-K |
| P1AF7468 | C2184200901-A | C121344880-H | C2184191702-K |
| P1AG0907 | C2184220339-A | C2184220338-P | C2184228838-M |
| P1AE2791 | C147684951-A (2x) | C147685020-P | C147684969-M |
| P1AG1545 | C2184220339-A | C2184220338-P | C2184229955-M |
| P1AD5053 | C08611380-A (3x) | C01478648-H | C08611149-K |

TABLE 4
Description and sequences of masks, release sites and IL2v present in the molecules
generated in Examples 5A and 5B.

| | | | | | SEQ N-term IL2v-C-term | SEQ |
|---|---|---|---|---|---|---|
| | TAPIR | | | description of mask, | mask OR N-term | ID |
| Molecule | ID | TYPE | ID | release sites and IL2v | mask-C-term IL2v | NO |
| P1AG9597 | C21842 | CHAIN | C218 | scFv mask (VH- | EVQLVESGGGLVQPGGSLRLSCAASGF | 71 |
| | 53067 | | 4253 | linker-VL)-linker- | TFSSYTLAWVRQAPGKGLEWVAAIDS | |
| | | | 067- | IL2v (linkers | SSYTYSPDTVRGRFTISRDNAKNSLYL | |
| | | | K | cleavable with | QMNSLRAEDTAVYYCARDSNWDALD | |
| | | | | PQARK release sites) | YWGQGTTVTVSSGGGGSGGPQARKG | |
| | | | | | GGGSGGGDIQMTQSPSSLSASVGDRVT | |
| | | | | | ITCKASQNVGTNVGWYQQKPGKAPKA | |
| | | | | | LIYSASFRYSGVPSRFSGSGSGTDFTLTI | |
| | | | | | SSLQPEDFATYYCQQYYTYPYTFGGGT | |
| | | | | | KVEIKGGGGSGGGGSPQARKGGGGSG | |
| | | | | | GGGSGGGGSGGGGSGGGAPASSSTKK | |
| | | | | | TQLQLEHLLLDLQMILNGINNYKNPKL | |
| | | | | | TRMLTAKFAMPKKATELKHLQCLEEE | |
| | | | | | LKPLEEVLNGAQSKNFHLRPRDLISNIN | |
| | | | | | VIVLELKGSETTFMCEYADETATIVEFL | |
| | | | | | NRWITFAQSIISTLT | |
| P1AG0929 | C21842 | CHAIN | C218 | scFv mask (VH- | EVQLVESGGGLVQPGGSLRLSCAASGF | 72 |
| | 20946 | | 4220 | linker-VL)-linker - | TFSSYTLAWVRQAPGKGLEWVAAIDS | |
| | | | 946- | IL2v (linkers non- | SSYTYSPDTVRGRFTISRDNAKNSLYL | |
| | | | K | cleavable) | QMNSLRAEDTAVYYCARDSNWDALD | |
| | | | | | YWGQGTTVTVSSGGGGSGGGGGGSG | |
| | | | | | GGGSGGGDIQMTQSPSSLSASVGDRVT | |
| | | | | | ITCKASQNVGTNVGWYQQKPGKAPKA | |
| | | | | | LIYSASFRYSGVPSRFSGSGSGTDFTLTI | |
| | | | | | SSLQPEDFATYYCQQYYTYPYTFGGGT | |
| | | | | | KVEIKGGGGSGGGGSGGGGSGGGGSG | |
| | | | | | GGGSGGGGSGGGGSGGGAPASSSTKK | |
| | | | | | TQLQLEHLLLDLQMILNGINNYKNPKL | |
| | | | | | TRMLTAKFAMPKKATELKHLQCLEEE | |
| | | | | | LKPLEEVLNGAQSKNFHLRPRDLISNIN | |
| | | | | | VIVLELKGSETTFMCEYADETATIVEFL | |
| | | | | | NRWITFAQSIISTLT | |
| P1AG9606 | C21842 | CHAIN | C218 | IL2v-linker-scFv | APASSSTKKTQLQLEHLLLDLQMILNGI | 73 |
| | 53077 | | 4253 | mask (VH-linker- | NNYKNPKLTRMLTAKFAMPKKATELK | |
| | | | 077- | VL) (linkers | HLQCLEEELKPLEEVLNGAQSKNFHLR | |
| | | | K | cleavable with | PRDLISNINVIVLELKGSETTFMCEYAD | |
| | | | | PQARK release sites) | ETATIVEFLNRWITFAQSIISTLTGGGGS | |
| | | | | | GGGGSPQARKGGGGSGGGGSGGGGSG | |
| | | | | | GGGSGGGEVQLVESGGGLVQPGGSLR | |
| | | | | | LSCAASGFTFSSYTLAWVRQAPGKGLE | |
| | | | | | WVAAIDSSSYTYSPDTVRGRFTISRDN | |
| | | | | | AKNSLYLQMNSLRAEDTAVYYCARDS | |
| | | | | | NWDALDYWGQGTTVTVSSGGGGSGG | |
| | | | | | PQARKGGGGSGGGDIQMTQSPSSLSAS | |
| | | | | | VGDRVTITCKASQNVGTNVGWYQQKP | |
| | | | | | GKAPKALIYSASFRYSGVPSRFSGSGSG | |
| | | | | | TDFTLTISSLQPEDFATYYCQQYYTYPY | |
| | | | | | TFGGGTKVEIK | |
| P1AG5740 | C21842 | CHAIN | C218 | IL2v-linker-scFv | APASSSTKKTQLQLEHLLLDLQMILNGI | 74 |
| | 36829 | | 4236 | mask (VH-linker- | NNYKNPKLTRMLTAKFAMPKKATELK | |
| | | | 829- | VL) (linkers non- | HLQCLEEELKPLEEVLNGAQSKNFHLR | |
| | | | K | cleavable) | PRDLISNINVIVLELKGSETTFMCEYAD | |
| | | | | | ETATIVEFLNRWITFAQSIISTLTGGGGS | |
| | | | | | GGGGSGGGGSGGGGSGGGGSGGGGS | |
| | | | | | GGGGSGGGGSGEVQLVESGGGLVQPG | |
| | | | | | GSLRLSCAASGFTFSSYTLAWVRQAPG | |
| | | | | | KGLEWVAAIDSSSYTYSPDTVRGRFTIS | |
| | | | | | RDNAKNSLYLQMNSLRAEDTAVYYCA | |
| | | | | | RDSNWDALDYWGQGTTVTVSSGGGG | |
| | | | | | SGGGGGGSGGGGSGGGDIQMTQSPSSL | |
| | | | | | SASVGDRVTITCKASQNVGTNVGWYQ | |
| | | | | | QKPGKAPKALIYSASFRYSGVPSRFSGS | |
| | | | | | GSGTDFTLTISSLQPEDFATYYCQQYYT | |
| | | | | | YPYTFGGGTKVEIK | |
| P1AG9607 | C21842 | CHAIN | C218 | linker-scFv mask | GGGGSGGGGSPQARKGGGGSGGGGSG | 75 |
| | 53078 | | 4253 | (VH-linker-VL) | GGGSGGGGSGGGEVQLVESGGGLVQP | |
| | | | 078- | (linkers cleavable | GGSLRLSCAASGFTFSSYTLAWVRQAP | |
| | | | H | with PQARK release | GKGLEWVAAIDSSSYTYSPDTVRGRFT | |
| | | | | sites) (IL2v on | ISRDNAKNSLYLQMNSLRAEDTAVYY | |
| | | | | separate | CARDSNWDALDYWGQGTTVTVSSGG | |
| | | | | C179973954-K | GGSGGPQARKGGGGSGGGDIQMTQSP | |
| | | | | chain) | SSLSASVGDRVTITCKASQNVGTNVG | |
| | | | | | WYQQKPGKAPKALIYSASFRYSGVPSR | |
| | | | | | FSGSGSGTDFTLTISSLQPEDFATYYCQ | |
| | | | | | QYYTYPYTFGGGTKVEIK | |
| P1AG9629 | C21842 | CHAIN | C218 | scFv mask (VH- | EVQLVESGGGLVQPGGSLRLSCAASGF | 76 |
| | 53107 | | 4253 | linker-VL)-linker- | TFSSYTLAWVRQAPGKGLEWVAAIDS | |
| | | | 107- | IL2v (linkers | SSYTYSPDTVRGRFTISRDNAKNSLYL | |
| | | | M | cleavable with | QMNSLRAEDTAVYYCARDSNWDALD | |
| | | | | PQARK release sites) | YWGQGTTVTVSSGGGGSGGPQARKG | |
| | | | | | GGGSGGGDIQMTQSPSSLSASVGDRVT | |
| | | | | | ITCKASQNVGTNVGWYQQKPGKAPKA | |
| | | | | | LIYSASFRYSGVPSRFSGSGSGTDFTLTI | |
| | | | | | SSLQPEDFATYYCQQYYTYPYTFGGGT | |
| | | | | | KVEIKGGGGSGGGGSPQARKGGGGSG | |
| | | | | | GGGSGGGGSGGGGSGGGAPASSSTKK | |
| | | | | | TQLQLEHLLLDLQMILNGINNYKNPKL | |
| | | | | | TRMLTAKFAMPKKATELKHLQCLEEE | |
| | | | | | LKPLEEVLNGAQSKNFHLRPRDLISNIN | |
| | | | | | VIVLELKGSETTFMCEYADETATIVEFL | |
| | | | | | NRWITFAQSIISTLT | |
| P1AG0905 | C21842 | CHAIN | C218 | scFv mask (VH- | EVQLVESGGGLVQPGGSLRLSCAASGF | 77 |
| | 28834 | | 4228 | linker-VL)-linker- | TFSSYTLAWVRQAPGKGLEWVAAIDS | |
| | | | 834- | IL2v (linkers non- | SSYTYSPDTVRGRFTISRDNAKNSLYL | |
| | | | M | cleavable) | QMNSLRAEDTAVYYCARDSNWDALD | |
| | | | | | YWGQGTTVTVSSGGGGSGGGGGGSG | |
| | | | | | GGGSGGGDIQMTQSPSSLSASVGDRVT | |
| | | | | | ITCKASQNVGTNVGWYQQKPGKAPKA | |
| | | | | | LIYSASFRYSGVPSRFSGSGSGTDFTLTI | |
| | | | | | SSLQPEDFATYYCQQYYTYPYTFGGGT | |
| | | | | | KVEIKGGGGSGGGGSGGGGSGGGGSG | |
| | | | | | GGGSGGGGSGGGGSGGGAPASSSTKK | |
| | | | | | TQLQLEHLLLDLQMILNGINNYKNPKL | |
| | | | | | TRMLTAKFAMPKKATELKHLQCLEEE | |
| | | | | | LKPLEEVLNGAQSKNFHLRPRDLISNIN | |
| | | | | | VIVLELKGSETTFMCEYADETATIVEFL | |
| | | | | | NRWITFAQSIISTLT | |
| P1AG9983 | C21842 | CHAIN | C218 | IL2v-linker-scFv | APASSSTKKTQLQLEHLLLDLQMILNGI | 78 |
| | 53819 | | 4253 | mask (VH-linker- | NNYKNPKLTRMLTAKFAMPKKATELK | |
| | | | 819- | VL) (linkers | HLQCLEEELKPLEEVLNGAQSKNFHLR | |
| | | | M | cleavable with | PRDLISNINVIVLELKGSETTFMCEYAD | |
| | | | | PQARK release sites) | ETATIVEFLNRWITFAQSIISTLTGGGGS | |
| | | | | | GGGGSPQARKGGGGSGGGGSGGGGSG | |
| | | | | | GGGSGGGEVQLVESGGGLVQPGGSLR | |
| | | | | | LSCAASGFTFSSYTLAWVRQAPGKGLE | |
| | | | | | WVAAIDSSSYTYSPDTVRGRFTISRDN | |
| | | | | | AKNSLYLQMNSLRAEDTAVYYCARDS | |
| | | | | | NWDALDYWGQGTTVTVSSGGGGSGG | |
| | | | | | PQARKGGGGSGGGDIQMTQSPSSLSAS | |
| | | | | | VGDRVTITCKASQNVGTNVGWYQQKP | |
| | | | | | GKAPKALIYSASFRYSGVPSRFSGSGSG | |
| | | | | | TDFTLTISSLQPEDFATYYCQQYYTYPY | |
| | | | | | TFGGGTKVEIK | |
| P1AG9984 | C21842 | CHAIN | C218 | IL2v-linker-scFv | APASSSTKKTQLQLEHLLLDLQMILNGI | 79 |
| | 53820 | | 4253 | mask (VH-linker- | NNYKNPKLTRMLTAKFAMPKKATELK | |
| | | | 820- | VL) (linkers non- | HLQCLEEELKPLEEVLNGAQSKNFHLR | |
| | | | M | cleavable) | PRDLISNINVIVLELKGSETTFMCEYAD | |
| | | | | | ETATIVEFLNRWITFAQSIISTLTGGGGS | |
| | | | | | GGGGSGGGGSGGGGSGGGGSGGGGS | |
| | | | | | GGGGSGGGEVQLVESGGGLVQPGGSL | |
| | | | | | RLSCAASGFTFSSYTLAWVRQAPGKGL | |
| | | | | | EWVAAIDSSSYTYSPDTVRGRFTISRDN | |
| | | | | | AKNSLYLQMNSLRAEDTAVYYCARDS | |
| | | | | | NWDALDYWGQGTTVTVSSGGGGSGG | |
| | | | | | GGGGSGGGGSGGGDIQMTQSPSSLSAS | |
| | | | | | VGDRVTITCKASQNVGTNVGWYQQKP | |
| | | | | | GKAPKALIYSASFRYSGVPSRFSGSGSG | |
| | | | | | TDFTLTISSLQPEDFATYYCQQYYTYPY | |
| | | | | | TFGGGTKVEIK | |
| P1AG9989 | C21842 | CHAIN | C218 | IL2v-linker-scFv | APASSSTKKTQLQLEHLLLDLQMILNGI | 80 |
| | 53824 | | 4253 | mask (VH-linker- | NNYKNPKLTRMLTAKFAMPKKATELK | |
| | | | 824- | VL) (linkers | HLQCLEEELKPLEEVLNGAQSKNFHLR | |
| | | | M | cleavable with | PRDLISNINVIVLELKGSETTFMCEYAD | |
| | | | | PQARK release sites) | ETATIVEFLNRWITFAQSIISTLTGGGGS | |
| | | | | | GGGGSPQARKGGGGSGGGGSGGGGSG | |
| | | | | | GGGSGGGEVQLVESGGGLVQPGGSLR | |
| | | | | | LSCAASGFTFSSYTLAWVRQAPGKGLE | |
| | | | | | WVAAIDSSSYTYSPDTVRGRFTISRDN | |
| | | | | | AKNSLYLQMNSLRAEDTAVYYCARDS | |
| | | | | | NWDALDYWGQGTTVTVSSGGGGSGG | |
| | | | | | PQARKGGGGSGGGDIQMTQSPSSLSAS | |
| | | | | | VGDRVTITCKASQNVGTNVGWYQQKP | |
| | | | | | GKAPKALIYSASFRYSGVPSRFSGSGSG | |
| | | | | | TDFTLTISSLQPEDFATYYCQQYYTYPY | |
| | | | | | TFGGGTKVEIK | |
| P1AG9990 | C21842 | CHAIN | C218 | IL2v-linker-scFv | APASSSTKKTQLQLEHLLLDLQMILNGI | 81 |
| | 53825 | | 4253 | mask (VH-linker- | NNYKNPKLTRMLTAKFAMPKKATELK | |
| | | | 825- | VL) (linkers non- | HLQCLEEELKPLEEVLNGAQSKNFHLR | |
| | | | M | cleavable) | PRDLISNINVIVLELKGSETTFMCEYAD | |
| | | | | | ETATIVEFLNRWITFAQSIISTLTGGGGS | |
| | | | | | GGGGSGGGGSGGGGSGGGGSGGGGS | |
| | | | | | GGGGSGGGEVQLVESGGGLVQPGGSL | |
| | | | | | RLSCAASGFTFSSYTLAWVRQAPGKGL | |
| | | | | | EWVAAIDSSSYTYSPDTVRGRFTISRDN | |
| | | | | | AKNSLYLQMNSLRAEDTAVYYCARDS | |
| | | | | | NWDALDYWGQGTTVTVSSGGGGSGG | |
| | | | | | GGGGSGGGGSGGGDIQMTQSPSSLSAS | |
| | | | | | VGDRVTITCKASQNVGTNVGWYQQKP | |
| | | | | | GKAPKALIYSASFRYSGVPSRFSGSGSG | |
| | | | | | TDFTLTISSLQPEDFATYYCQQYYTYPY | |
| | | | | | TFGGGTKVEIK | |
| P1AG9994 | C21842 | CHAIN | C218 | linker-scFv mask | GGGGSGGGGSPQARKGGGGSGGGGSG | 82 |
| | 53830 | | 4253 | (VH-linker-VL) | GGGSGGGGSGGGEVQLVESGGGLVQP | |
| | | | 830- | (linkers cleavable | GGSLRLSCAASGFTFSSYTLAWVRQAP | |
| | | | M | with PQARK release | GKGLEWVAAIDSSSYTYSPDTVRGRFT | |
| | | | | sites) (IL2v on | ISRDNAKNSLYLQMNSLRAEDTAVYY | |
| | | | | separate | CARDSNWDALDYWGQGTTVTVSSGG | |
| | | | | C2184253831-P | GGSGGPQARKGGGGSGGGDIQMTQSP | |
| | | | | chain) | SSLSASVGDRVTITCKASQNVGTNVG | |
| | | | | | WYQQKPGKAPKALIYSASFRYSGVPSR | |
| | | | | | FSGSGSGTDFTLTISSLQPEDFATYYCQ | |
| | | | | | QYYTYPYTFGGGTKVEIK | |
| P1AG9995 | C21842 | CHAIN | C218 | linker-scFv mask | GGGGSGGGGGGGGSGGGGSGGGGS | 83 |
| | 53832 | | 4253 | (VH-linker-VL) | GGGGSGGGGSGGGEVQLVESGGGLVQ | |
| | | | 832- | (linkers non- | PGGSLRLSCAASGFTFSSYTLAWVRQA | |
| | | | M | cleavable) (IL2v on | PGKGLEWVAAIDSSSYTYSPDTVRGRF | |
| | | | | separate | TISRDNAKNSLYLQMNSLRAEDTAVY | |
| | | | | C2184253831-P | YCARDSNWDALDYWGQGTTVTVSSG | |
| | | | | chain) | GGGSGGGGGGSGGGGSGGGDIQMTQS | |
| | | | | | PSSLSASVGDRVTITCKASQNVGTNVG | |
| | | | | | WYQQKPGKAPKALIYSASFRYSGVPSR | |
| | | | | | FSGSGSGTDFTLTISSLQPEDFATYYCQ | |
| | | | | | QYYTYPYTFGGGTKVEIK | |
| P1AF6882 | C21842 | CHAIN | C218 | scFv mask (VH- | EVQLVESGGGLVQPGGSLRLSCAASGF | 84 |
| | 20944 | | 4220 | linker-VL)-linker- | TFSSYTLAWVRQAPGKGLEWVAAIDS | |
| | | | 944- | IL2v (linkers | SSYTYSPDTVRGRFTISRDNAKNSLYL | |
| | | | K | cleavable with | QMNSLRAEDTAVYYCARDSNWDALD | |
| | | | | PMAKK release | YWGQGTTVTVSSGGGGSGGPMAKKG | |
| | | | | sites) | GGGSGGGDIQMTQSPSSLSASVGDRVT | |
| | | | | | ITCKASQNVGTNVGWYQQKPGKAPKA | |
| | | | | | LIYSASFRYSGVPSRFSGSGSGTDFTLTI | |
| | | | | | SSLQPEDFATYYCQQYYTYPYTFGGGT | |
| | | | | | KVEIKGGGGSGGGGSPMAKKGGGGSG | |
| | | | | | GGGSGGGGSGGGGSGGGAPASSSTKK | |
| | | | | | TQLQLEHLLLDLQMILNGINNYKNPKL | |
| | | | | | TRMLTAKFAMPKKATELKHLQCLEEE | |
| | | | | | LKPLEEVLNGAQSKNFHLRPRDLISNIN | |
| | | | | | VIVLELKGSETTFMCEYADETATIVEFL | |
| | | | | | NRWITFAQSIISTLT | |
| P1AG0907 | C21842 | CHAIN | C218 | scFv mask (VH- | EVQLVESGGGLVQPGGSLRLSCAASGF | 85 |
| | 28838 | | 4228 | linker-VL)-linker- | TFSSYTLAWVRQAPGKGLEWVAAIDS | |
| | | | 838- | IL2v (linkers | SSYTYSPDTVRGRFTISRDNAKNSLYL | |
| | | | M | cleavable with | QMNSLRAEDTAVYYCARDSNWDALD | |
| | | | | PMAKK release | YWGQGTTVTVSSGGGGSGGPMAKKG | |
| | | | | sites) | GGGSGGGDIQMTQSPSSLSASVGDRVT | |
| | | | | | ITCKASQNVGTNVGWYQQKPGKAPKA | |
| | | | | | LIYSASFRYSGVPSRFSGSGSGTDFTLTI | |
| | | | | | SSLQPEDFATYYCQQYYTYPYTFGGGT | |
| | | | | | KVEIKGGGGSGGGGSPMAKKGGGGSG | |
| | | | | | GGGSGGGGSGGGGSGGGAPASSSTKK | |
| | | | | | TQLQLEHLLLDLQMILNGINNYKNPKL | |
| | | | | | TRMLTAKFAMPKKATELKHLQCLEEE | |
| | | | | | LKPLEEVLNGAQSKNFHLRPRDLISNIN | |
| | | | | | VIVLELKGSETTFMCEYADETATIVEFL | |
| | | | | | NRWITFAQSIISTLT | |
| P1AG1545 | C21842 | CHAIN | C218 | scFv mask (VH- | EVQLVESGGGLVQPGGSLRLSCAASGF | 86 |
| | 29955 | | 4229 | linker-VL)-linker- | TFSSYTLAWVRQAPGKGLEWVAAIDS | |
| | | | 955- | IL2v (linkers | SSYTYSPDTVRGRFTISRDNAKNSLYL | |
| | | | M | cleavable with | QMNSLRAEDTAVYYCARDSNWDALD | |
| | | | | YAARKGGI release | YWGQGTTVTVSSGGGGSGGYAARKG | |
| | | | | sites) | GIGGGGSGGGDIQMTQSPSSLSASVGD | |
| | | | | | RVTITCKASQNVGTNVGWYQQKPGKA | |
| | | | | | PKALIYSASFRYSGVPSRFSGSGSGTDF | |
| | | | | | TLTISSLQPEDFATYYCQQYYTYPYTFG | |
| | | | | | GGTKVEIKGGGGSGGGGSYAARKGGI | |
| | | | | | GGGGSGGGGSGGGGSGGGGSGGGAP | |
| | | | | | ASSSTKKTQLQLEHLLLDLQMILNGIN | |
| | | | | | NYKNPKLTRMLTAKFAMPKKATELKH | |
| | | | | | LQCLEEELKPLEEVLNGAQSKNFHLRP | |
| | | | | | RDLISNINVIVLELKGSETTFMCEYADE | |
| | | | | | TATIVEFLNRWITFAQSIISTLT | |

TABLE 5
VH and VL sequences and respective specificities of molecules generated in Examples
5A and 5B.

| | | | | SEQ ID | in | in |
|---|---|---|---|---|---|---|
| SEQ VL | SEQ VH | specificity | paratop ID | NO | CHAIN | MOLECULE |
| DIVMTQSPDSLAV | | human | FV000363 | 87 | C02011 | P1AG9597, |
| SLGERATINCKAS | | PD1 | | | 851-A | P1AG0929, |
| ESVDTSDNSFIHW | | | | | | P1AG3071, |
| YQQKPGQSPKLLI | | | | | | P1AG9606, |
| YRSSTLESGVPDR | | | | | | P1AG5740, |
| FSGSGSGTDFTLTI | | | | | | P1AG5741 = |
| SSLQAEDVAVYY | | | | | | P1AA7146, |
| CQQNYDVPWTFG | | | | | | P1AG9607 |
| QGTKVEIK | | | | | | |
| | EVQLLESGGGL | human | FV000363 | 88 | C21842 | P1AG9597, |
| | VQPGGSLRLSC | PD1 | | | 30856- | P1AG0929, |
| | AASGFSFSSYT | | | | H | P1AG3071, |
| | MSWVRQAPGK | | | | | P1AG9606, |
| | GLEWVATISGG | | | | | P1AG5740, |
| | GRDIYYPDSVK | | | | | P1AG5741 |
| | GRFTISRDNSK | | | | | P1AA7146, |
| | NTLYLQMNSL | | | | | P1AG9607 |
| | RAEDTAVYYC | | | | | |
| | VLLTGRVYFAL | | | | | |
| | DSWGQGTLVT | | | | | |
| | VSS | | | | | |
| KIVLTQSPASLPVS | | human | FV000638 | 89 | C21842 | P1AG9629, |
| LGQRATISCRASE | | PD1 | | | 20339- | P1AG0905, |
| SVDTSDNSFIHWY | | | | | A | P1AG3108, |
| QQRPGQSPKLLIY | | | | | | P1AG9983, |
| RSSTLESGVPARFS | | | | | | P1AG9984, |
| GSGSRTDFTLTIDP | | | | | | P1AG7552, |
| VEADDVATYYCQ | | | | | | P1AG0907, |
| QNYDVPWTFGGG | | | | | | P1AE2791, |
| TKLEIK | | | | | | P1AG1545 |
| | EVILVESGGGL | human | FV000638 | 90 | C21842 | P1AG9629, |
| | VKPGGSLKLSC | PD1 | | | 53107- | P1AG0905, |
| | AASGFSFSSYT | | | | M, | P1AG3108, |
| | MSWVRQTPEK | | | | C21842 | P1AG9983, |
| | RLDWVATISGG | | | | 20338- | P1AG9984, |
| | GRDIYYPDSVK | | | | P | P1AG7552, |
| | GRFTISRDNAK | | | | | P1AG0907, |
| | NTLYLEMSSLM | | | | | P1AE2791, |
| | SEDTALYYCVL | | | | | P1AG1545 |
| | LTGRVYFALDS | | | | | |
| | WGQGTSVTVS | | | | | |
| | S | | | | | |
| DIVMTQGTLPNPV | | murine | FV001624 | 91 | C01486 | P1AG9630, |
| PSGESVSITCRSSK | | PD1 | | | 562-A | P1AG0908, |
| SLLYSDGKTYLN | | | | | | P1AG3109, |
| WYLQRPGQSPQL | | | | | | P1AG9989 |
| LIYWMSTRASGVS | | | | | | P1AG9990, |
| DRFSGSGSGTDFT | | | | | | P1AG9991, |
| LKISGVEAEDVGI | | | | | | P1AG9994, |
| YYCQQGLEFPTFG | | | | | | P1AG9995 |
| GGTKLELK | | | | | | |
| | EVQLQESGPGL | murine | FV001624 | 92 | C21842 | P1AG9630, |
| | VKPSQSLSLTC | PD1 | | | 20341- | P1AG0908, |
| | SVTGYSITSSYR | | | | P | P1AG3109, |
| | WNWIRKFPGN | | | | | P1AG9989, |
| | RLEWMGYINS | | | | | P1AG9990, |
| | AGISNYNPSLK | | | | | P1AG9991, |
| | RRISITRDTSKN | | | | | P1AG9994, |
| | QFFLQVNSVTT | | | | | P1AG9995 |
| | EDAATYYCAR | | | | | |
| | SDNMGTTPFTY | | | | | |
| | WGQGTLVTVS | | | | | |
| | S | | | | | |
| DVQITQSPSSLSAS | | human | FV005131 | 93 | C21842 | P1AF6882, |
| VGDRVTITCRTSR | | CD8 | | | 00901- | P1AF6883, |
| SISQYLAWYQEKP | | | | | A | P1AF7468, |
| GKTNKLLIYSGST | | | | | | |
| LQSGIPSRFSGSGS | | | | | | |
| GTDFTLTISSLQPE | | | | | | |
| DFATYYCQQVNE | | | | | | |
| FPPTFGQGTKVEI | | | | | | |
| K | | | | | | |
| | EVQLVQSGAE | human | FV005131 | 94 | C12134 | P1AF6882, |
| | VKKPGASVKV | CD8 | | | 4880-H | P1AF6883, |
| | SCKASGFNIKD | | | | | P1AF7468, |
| | TYIHWVRQAP | | | | | |
| | GQGLEWIGRID | | | | | |
| | PANDNTLYASK | | | | | |
| | FQGRATITADT | | | | | |
| | STSTAYLELSSL | | | | | |
| | RSEDTAVYYC | | | | | |
| | GRGYGYYVFD | | | | | |
| | HWGQGTLVTV | | | | | |
| | SS | | | | | |
| QAVVTQEPSLTVS | | human | FV000439 | 95 | C08611 | P1AD5053 |
| PGGTVTLTCGSST | | CD3e | | | 380-A | |
| GAVTTSNYANWV | | | | | | |
| QEKPGQAFRGLIG | | | | | | |
| GTNKRAPGTPARF | | | | | | |
| SGSLLGGKAALTL | | | | | | |
| SGAQPEDEAEYYC | | | | | | |
| ALWYSNLWVFGG | | | | | | |
| GTKLTVL | | | | | | |
| | EVQLLESGGGL | human | FV000439 | 96 | C08611 | P1AD5053 |
| | VQPGGSLRLSC | CD3e | | | 149-K | |
| | AASGFTFSTYA | | | | | |
| | MNWVRQAPGK | | | | | |
| | GLEWVSRIRSK | | | | | |
| | YNNYATYYAD | | | | | |
| | SVKGRFTISRD | | | | | |
| | DSKNTLYLQM | | | | | |
| | NSLRAEDTAVY | | | | | |
| | YCVRHGNFGN | | | | | |
| | SYVSWFAYWG | | | | | |
| | QGTLVTVSS | | | | | |
| QAVVTQEPSLTVS | | human | FV000670 | 97 | C08611 | P1AD5053 |
| PGGTVTLTCGSST | | FolRI | | | 380-A | |
| GAVTTSNYANWV | | | | | | |
| QEKPGQAFRGLIG | | | | | | |
| GTNKRAPGTPARF | | | | | | |
| SGSLLGGKAALTL | | | | | | |
| SGAQPEDEAEYYC | | | | | | |
| ALWYSNLWVFGG | | | | | | |
| GTKLTVL | | | | | | |
| | EVQLVESGGGL | human | FV000670 | 98 | C08611 | P1AD5053 |
| | VKPGGSLRLSC | FolR1 | | | 149-K, | |
| | AASGFTFSNAW | | | | C01478 | |
| | MSWVRQAPGK | | | | 648-H | |
| | GLEWVGRIKSK | | | | | |
| | TDGGTTDYAA | | | | | |
| | PVKGRFTISRD | | | | | |
| | DSKNTLYLQM | | | | | |
| | NSLKTEDTAVY | | | | | |
| | YCTTPWEWSW | | | | | |
| | YDYWGQGTLV | | | | | |
| | TVSS | | | | | |

### Example 6A

### Binding of TA PD1-IL2v Constructs to T Cells

Three formats of TA PD1-IL2v molecules and respective controls were assessed for their capacity to bind to activated PD1 positive CD8 T cells and CD4 T cells within PBMCs in comparison to the respective unmasked molecules.

PBMCs were purchased from Biomex from a healthy donor (Lot 5000729PB). The PBMCs were stimulated for three days with CD3 and CD28 to induce upregulation of PD1 on T cells. Therefore, PBMCs were seeded in medium (RPMI1640, 10% FCS, 2 mM Glutamine) into cell culture flasks that were coated for 1 hour at 37° C. with 1 μg/ml CD3 (clone OKT3, 302914, BioLegend). CD28 was added in solution to the PBMCs at a concentration of 1 μg/ml (clone CD28.2, 302914, BioLegend). After three days, PBMCs were harvested and transferred into a 96 well round bottom plate (200′000 cells per well). The cells were washed with FACS buffer (PBS, 2% FBS, 5 mM EDTA, 0.025% NaN3) and stained with 30 μl of corresponding TA PD1-IL2v constructs in FACS buffer for 30 min at 4° C. Prior to the staining, 20 μg of the TA PD1-IL2v constructs were digested with 4 μl Matriptase (Enzo ˜2.5 U/μl, ALX-201-246-U25, Lot 12152015, or without Matriptase for the unmasked controls) for 2 hours at 37° C. in 40 μl Matriptase buffer (50 mM Tris, 50 mM NaCl, 0.01% Tween 20, pH 9.0). After the staining, the cells were washed twice with FACS buffer to remove unbound molecules. Then 30 μl of the diluted PE anti-human Fc specific secondary antibody (1:50 dilution, 109-116-170, Jackson ImmunoResearch) was added to the cells. After 30 min incubation at 4° C. the cells were washed twice with FACS buffer. To detect T cells, PBMCs were stained with 30 μl of a mixture of CD3 PE/Cyanine7 (clone UCHT1, 300420, BioLegend), CD4 FITC (clone RPA-T4, 300528, BioLegend) and CD8 APC/Cyanine7 (clone HIT8a, 300926, BioLegend) for 30 min at 4° C. The unbound antibodies were removed by washing twice with FACS buffer. Finally the cells resuspended in 150 μl FACS buffer and measured using a BD Fortessa gating on CD3+CD4+ cells (CD4 T cells) and CD3+CD8+ cells (CD8 T cells).

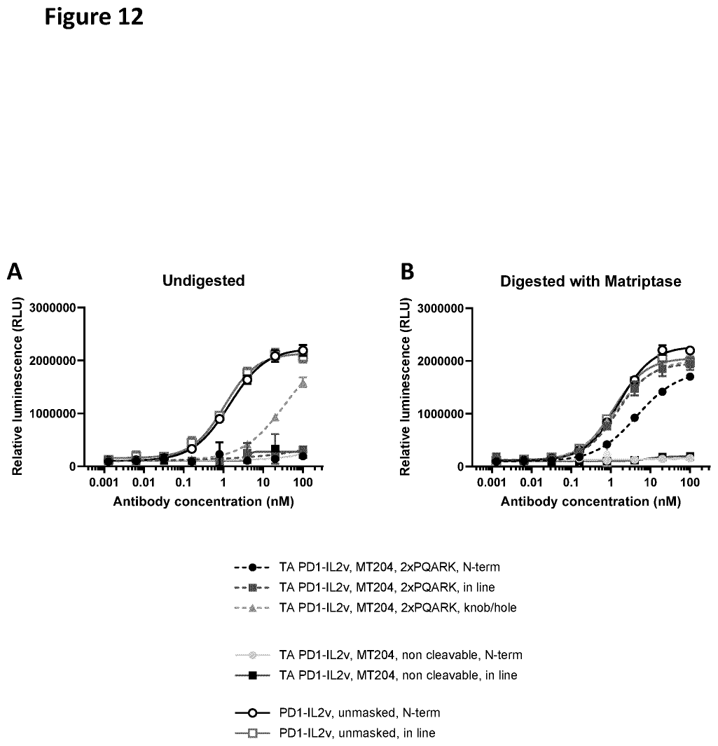

The tested TA PD1-IL2v constructs bind similarly well to PD1 on activated CD4 and activated CD8 T cells compared to the respective unmasked molecules. The N-term constructs, which contain only one PD1 binding Fab, show an approximately two fold higher binding capacity than the “in line” and knob/hole formats, which contain two PD1 binding Fabs (FIGS. 11A and 11B).

### Example 6B

### NK92 Proliferation Induced by TA PD1-IL2v Constructs

The induction of proliferation of NK92 cells by the different formats of TA PD1-IL2v, N-term, “in line” and knob/hole were tested. As controls, the two respective unmasked control molecules and the non-cleavable controls of the N-term and in line format were included. All molecules were tested undigested and upon digestion with recombinant Matriptase.

NK92 cells were harvested, counted and assessed for viability. Cells were washed three times with PBS to remove residual IL2. The washed NK92 cells were re-suspended in fresh medium (advanced RPMI1640, 2% FCS, 1% Glutamine) without IL2 to 160′000 cells per ml and 12.5 μl of the cell suspension was transferred in a 384-well cell culture treated flat bottom plate. 10 μg of the TA PD1-IL2v constructs were digested with 2 μl Matriptase (Enzo ˜2.5 U/μl, ALX-201-246-U25, Lot 12152015 or without Matriptase as undigested control) for 2 hours at 37° C. in 20 μl Matriptase buffer (50 mM Tris, 50 mM NaCl, 0.01% Tween 20, pH 9.0) and 12.5 μl of the antibodies were added per well to reach a final volume of 25 μl per well. The plate was incubated for 3 days in the incubator. After 3 days, the CellTiter-Glo (G7571, Promega) reagents and the cell culture plate were equilibrated to room temperature. The CellTiter-Glo solution was prepared as described in the manufacturer's instructions and 25 μl of the solution were added to each well. After 10 min of incubation, remaining aggregates were re-suspended by pipetting and 40 μl of the mixture were transferred to a white flat bottom plate. The luminescence was measured with a Tecan Spark 10M multimode reader.

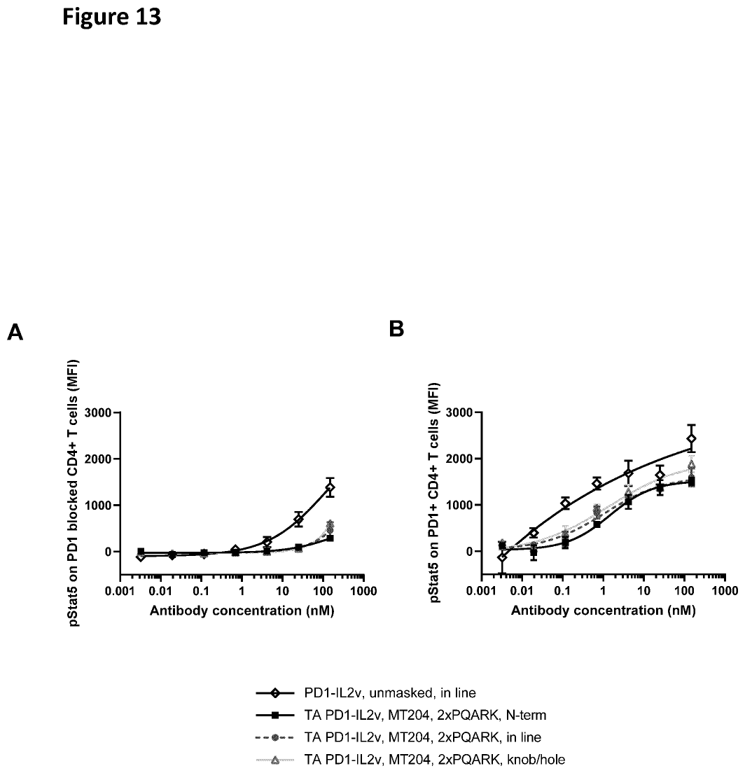

The undigested TA PD1-IL2v “in line” and N-term formats do not induce proliferation. The knob/hole format remains active but the activity is reduced compared to the unmasked control molecule. Upon digestion with recombinant matriptase the “in line” and knob/hole formats regain full activity, the activity of the N-term format is slightly reduced compared to the unmasked control. The non-cleavable molecules remain completely inactive (FIGS. 12A and 12B).

### Example 6C

### STAT5 Phosphorylation Induced by TA PD1-IL2v Constructs

The induction of STAT5 phosphorylation in activated PD1 positive CD4 T cells upon treatment with Matriptase digested TA PD1-IL2v constructs was tested.

PBMCs were bought from Biomex from a healthy donor (Lot 5000729PB). CD4 positive T cells were isolated using the human CD4 MicroBeads (130-045-101, Miltenyi Biotec) as described in the manufacturer's instructions. The CD4 positive T cells were stimulated for 4 days with CD3 and CD28 to induce upregulation of PD1. Therefore, CD4 positive T cells were seeded in medium (RPMI1640, 10% FCS, 2 mM Glutamine) into cell culture flasks that were coated for 1 h at 37° C. with 1 μg/ml CD3 (clone OKT3, 302914, BioLegend). CD28 was added in solution to the CD4 positive T cells at a concentration of 1 μg/ml (clone CD28.2, 302914, BioLegend). One day before the STAT5 phosphorylation assay set up, the TA PD1-IL2v constructs were digested with Matriptase (Enzo ˜2.5 U/μl, ALX-201-246-U25, Lot 12152015, or without Matriptase for the unmasked control). Therefore, 15 μg of the TA PD1-IL2v constructs were digested with 3 μl Matriptase for 2 hours at 37° C. in 30 μl Matriptase buffer (50 mM Tris, 50 mM NaCl, 0.01% Tween 20, pH 9.0). The digested constructs were incubated overnight in medium (RPMI1640, 10% FCS, 1% Glutamine) at 37° C. One half of the activated CD4 positive T cells were labeled with CFSE (5 (6)-Carboxyfluorescein diacetate N-succinimidyl ester, 21888, Sigma-Aldrich). Therefore, 30 million T cells were washed once with PBS. In parallel, the CFSE stock solution (2 mM in DMSO) was diluted 1:20 in prewarmed PBS. T cells were resuspended in 30 ml prewarmed PBS, 30 μl of the CFSE solution was added and the cells were mixed immediately. For an optimal labeling the cells were incubated for 15 min at 37° C. Then 10 ml prewarmed medium (RPMI1640, 10% FCS, 1% Glutamine) were added to stop the labeling reaction. The cells were spun down for 10 min at 400 g and resuspended in 20 ml fresh medium and incubated for additional 30 min at 37° C. Finally, the cells were washed once with medium and resuspended in fresh medium at 4 million cells per ml. The other half of the activated CD4 positive T cells were stained with PD1 IgG (in house production, human PD1 0376 binder, P1AD4476) to block the PD1 receptor. Therefore, the T cells were washed with medium (RPMI1640, 10% FCS, 2 mM Glutamine) and incubated with 10 μg/ml PD1-IgG in 30 μl medium for 30 min at room temperature. The cells were washed once with medium and resuspended in fresh medium at 4 million cells per ml. Equal amount (100′000 cells each) of PD1 blocked and PD1 positive cells were seeded to a 96 well round bottom plate. The plates were centrifuged at 300 g for 10 min and the supernatant was removed. The cells were re-suspended in 100 μl medium containing the TA PD1-IL2v molecules and stimulated for 20 min at 37° C. To preserve the phosphorylation status, the cells were immediately fixed after stimulation with equal amount of pre-warmed Cytofix buffer (554655, BD Bioscience) for 10 min at 37° C. Afterwards the plates were centrifuged for 10 min at 300 g and the supernatant was removed. To allow intracellular staining, the cells were permeabilized in 200 μl Phosflow Perm buffer III (558050, BD Bioscience) for 30 min at 4° C. Then the cells were washed twice with 150 μl cold FACS buffer and stained with 30 μl of a mixture of CD4 PE/Cyanine7 (clone SK3, 557852, BD) and stat5 AF647 (clone pY694, 612599, BD) for 30 min at 4° C. The unbound antibodies were removed by washing twice with FACs buffer then resuspended in 150 μl FACS buffer per well. The analysis was performed using a BD Fortessa flow cytometer gating on PD1 positive CD4 T cells (CFSE positive) and PD1 blocked (CFSE negative) cells.

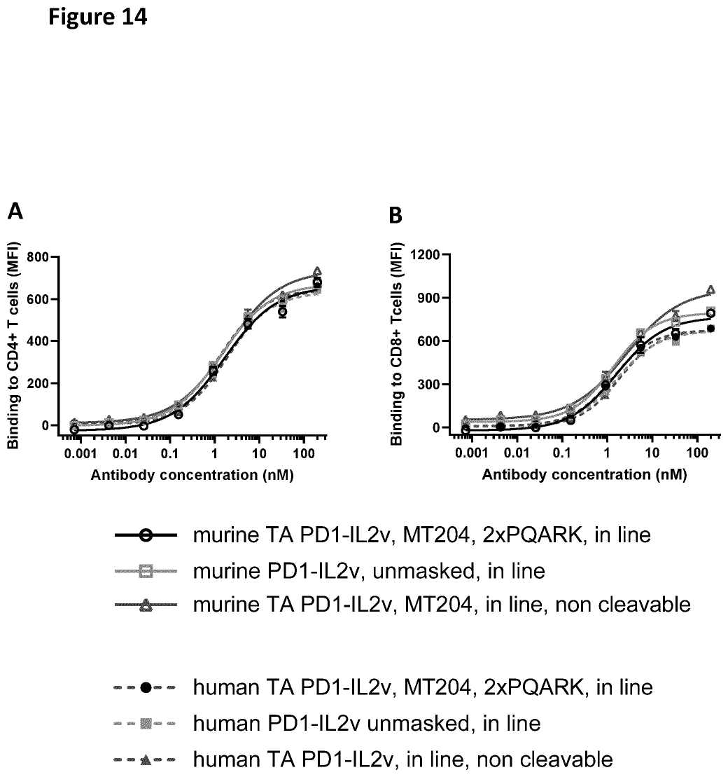

On CD4 T cells with blocked PD1, there is no activation by the masked digested constructs and only minimal activation by the unmasked construct (FIG. 13A). On the PD1 positive CD4 T cells, all tested constructs induce phosphorylation of STAT5, the unmasked construct has the highest activity followed by the other three, which show similar activities (FIG. 13B).

### Example 6D

### Binding of Murine TA PD1-IL2v Constructs to T Cells

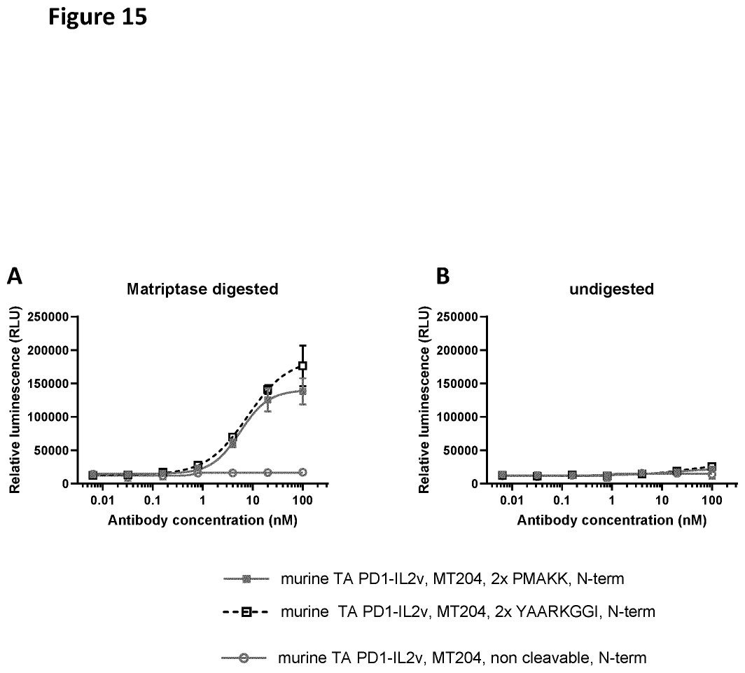

Binding of the murine TA PD1-IL2v, MT204, 2×PQARK “in line” construct, the non-cleavable control and the unmasked control to activated T cells was tested and compared to the respective human constructs (FIG. 14).

PBMCs were bought from Biomex from a healthy donor (Lot 5000899PB). The PBMCs were stimulated for 3 days with CD3 and CD28 to induce upregulation of PD1 on T cells. Therefore, PBMCs were seeded in medium (RPMI1640, 10% FCS, 2 mM Glutamine) into cell culture flasks that were coated for 1 h at 37° C. with 1 μg/ml CD3 (clone OKT3, 302914, BioLegend). CD28 was added in solution to the PBMCs at a concentration of 1 μg/ml (clone CD28.2, 302914, BioLegend). After three days, PBMCs were harvested and transferred into a 96 well round bottom plate (200′000 cells per well). The cells were washed with FACS buffer (PBS, 2% FBS, 5 mM EDTA, 0.025% NaN3) and stained with 30 μl of corresponding TA PD1-IL2v constructs in FACS buffer for 30 min at 4° C. The cells were washed twice with FACS buffer to remove unbound molecules. Then 30 μl of the diluted FITC anti-mouse Fc specific secondary antibody (1:50 dilution, 115-096-071, Jackson ImmunoResearch) or for the human constructs the diluted FITC anti-human Fc specific secondary antibody (1:50 dilution, 115-096-098, Jackson ImmunoResearch) was added to the cells. After 30 min incubation at 4° C. the cells were washed twice with FACS buffer. To detect T cells, PBMCs were stained with 30 μl of a mixture of CD3 PE/Cyanine7 (clone UCHT1, 300420, BioLegend), CD4 PE (clone RPA-T4, 300508, BioLegend) and CD8 APC (clone SK1, 344722, BioLegend) for 30 min at 4° C. The unbound antibodies were removed by washing twice with FACS buffer. Finally the cells were resuspended in 150 μl FACs buffer and measured using a BD Fortessa gating on CD3+CD4+ cells (CD4 T cells) and CD3+CD8+ cells (CD8 T cells).

The murine constructs bind equally well than the human constructs to activated PD1 positive CD4 (FIG. 14A) and CD8 T cells (FIG. 14B).

### Example 6E

### NK92 Proliferation Induced by Murine TA PD1-IL2v Constructs

A set of murine TA PD1-IL2v constructs was analyzed for the ability to induce proliferation of NK92 cells, which included a comparison of Matriptase digested and undigested constructs.

NK92 cells were harvested, counted and assessed for viability. Cells were washed three times with PBS to remove residual IL2. The washed NK92 cells were re-suspended in fresh medium (advanced RPMI1640, 2% FCS, 1% Glutamine) without IL2 to 160′000 cells per ml and 12.5 μl of the cell suspension was transferred in a 384-well cell culture treated flat bottom plate. 10 μg of the TA PD1-IL2v constructs were digested with 2 μl Matriptase (Enzo ˜2.5 U/μl, ALX-201-246-U25, Lot 12152015 or without Matriptase as undigested control) for 2 hours at 37° C. in 20 μl Matriptase buffer (50 mM Tris, 50 mM NaCl, 0.01% Tween 20, pH 9.0) and 12.5 μl of the antibodies were added per well to reach a final volume of 25 μl per well. The plate was incubated for 3 days in the incubator. After 3 days, the CellTiter-Glo (G7571, Promega) reagents and the cell culture plate were equilibrated to room temperature. The CellTiter-Glo solution was prepared as described in the manufacturer's instructions and 25 μl of the solution were added to each well. After 10 min of incubation, remaining aggregates were re-suspended by pipetting and 40 μl of the mixture were transferred to a white flat bottom plate. The luminescence was measured with a Tecan Spark 10M multimode reader.

The murine TA PD1-IL2v, MT204 N-term constructs with 2×PMAKK, 2×YAARKGGI or 2×PQARK as well as non-cleavable showed no activity. After digestion with Matriptase all three constructs that comprise a cleavage site regain activity whereas the non-cleavable construct remains inert (FIGS. 5A and 5B). The activity of the digested murine construct with 2×PQARK construct is comparable to the respective digested human construct (FIG. 5C). In addition, the murine TA PD1-IL2v, MT204, 2×PQARK, in line construct digested with Matriptase were tested showing a comparable activity to the respective human construct after digestion and the non-masked constructs. The non-cleavable control molecules have no activity on NK92 cells (FIG. 5D).

TABLE 6
Human constructs and IDs tested in Example 6A-6E.

| Name | ID |
|---|---|
| human TA PD1-IL2v, MT204, 2xPQARK, N-term | P1AG9597-007 |
| human TA PD1-IL2v, MT204, non cleavable, N-term | P1AG0929-004 |
| human PD1-IL2v, unmasked, N-term | P1AG3071-004 |
| human TA PD1-IL2v, MT204, 2xPQARK, knob/hole | P1AG9607-006 |
| human TA PD1-IL2v, MT204, 2xPQARK, in line | P1AG9606-008 |
| human TA PD1-IL2v, MT204, non cleavable, in line | P1AG5740-004 |
| human PD1-IL2v, unmasked, in line | P1AA7146-006 |

TABLE 7
Murine constructs and IDs tested in Examples 6A-6E.

| Name | ID |
|---|---|
| murine TA PD1-IL2v, MT204, 2xPQARK, in line | P1AG9983-001 |
| murine PD1-IL2v, unmasked, in line | P1AG7552-006 |
| murine TA PD1-IL2v, MT204, in line, non cleavable | P1AG9984-001 |
| murine TA PD1-IL2v, MT204, 2xPMAKK, N-term | P1AG0907-004 |
| murine TA PD1-IL2v, MT204, 2x YAARKGGI, N-term | P1AG1545-005 |
| murine TA PD1-IL2v, MT204, non cleavable, N-term | P1AG0905-005 |
| murine TA PD1-IL2v, MT204, 2x PQARK, N-term | P1AG9629-001 |

### Example 7

### In Vivo Efficacy of Murinized TA-PD1-IL2v Immuno-Conjugates, in a Syngeneic Model of Mouse Tumor Cell Line (KPC-4662 Subcutaneous Syngeneic Model)

The murinized TA-PD1-IL2v immuno-conjugates were tested in the mouse pancreatic cell line KPC-4662, injected subcutaneously into Black 6-huPD1 transgenic mice.

The KPC-4662 pancreatic carcinoma cells were originally obtained from Pennsylvania University (Pennsylvania, USA) and after expansion deposited in the Roche-Glycart internal cell bank. The tumor cell line was routinely cultured in DMEM containing 10% FCS (Gibco) and G418 (Geniticin; Gibco) at 37° C. in a water-saturated atmosphere at 5% CO2. Passage 8 was used for transplantation, at a viability of 93.8%. 3×10⁵ cells per animal were injected subcutaneously in 100 μl of RPMI cell culture medium (Gibco) into the flank of mice using a 1 ml tuberculin syringe (BD Biosciences, Germany).

Female Black 6-huPD1 mice, aged 10-11 weeks at the start of the experiment (bred at Charles Rivers, Lyon, France) were maintained under specific-pathogen-free condition with daily cycles of 12 h light/12 h darkness according to committed guidelines (GV-Solas; Felasa; TierschG). The experimental study protocol was reviewed and approved by local government (P 181/2020). After arrival, animals were maintained for one week to get accustomed to the new environment and for observation. Continuous health monitoring was carried out on a regular basis.

Mice were injected subcutaneously on study day 0 with 3×10⁵ of KPC-4662 cells, randomized and weighed. Two weeks after the tumor cell injection (tumor volume >200 mm³), mice were injected i.v. with TA-PD1-IL-2v PMAKK cleavable linker, TA-PD1-IL-2v YAARKGGI cleavable linker, TA-PD1-IL-2v non cleavable linker, TA-PD1-IL-2v non masked or PD1-IL-2v once weekly for two weeks. All mice were injected i.v. with 200 μl of the appropriate solution. The mice in the Vehicle group were injected with Histidine Buffer. To obtain the proper amount of immunoconjugate per 200 μl, the stock solutions were diluted with Histidine Buffer when necessary.

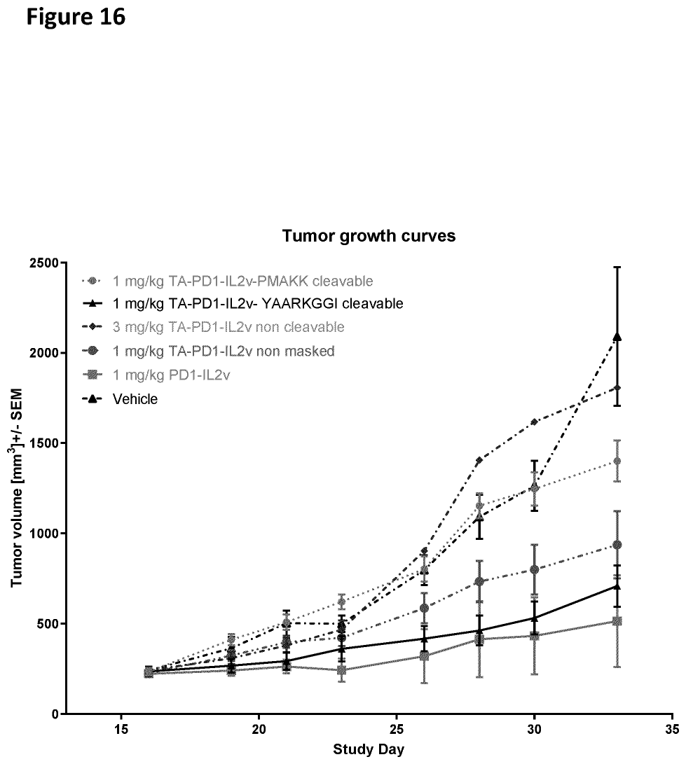

FIG. 16 shows that the TA-PD-IL2v YARRKGGI mediated superior efficacy in terms of tumor growth inhibition compared to vehicle, non-cleavable and non-masked Mab single agent groups. The TA-PD-IL2v YARRKGGI cleavable linker showed similar tumor growth inhibition as the PD1-IL2v group.

TABLE 8

| | Dose/ | Concentration |
|---|---|---|
| Compound | mouse | (mg/mL) |
| TA-PD1-IL2v PMAKK cleavable, | 20 μg | 2.65 (=stock solution) |
| P1AG0907 | | |
| TA-PD1-IL2v YAARKGGI cleavable, | 20 μg | 1.45 (=stock solution) |
| P1AG1545 | | |
| TA-PD1-IL2v non-cleavable, | 60 μg | 3.9 (=stock solution) |
| P1AG0905 | | |
| TA-PD1-IL2v non-masked, P1AG3108 | 20 μg | 3.5 (=stock solution) |
| PD1-IL2v, P1AE2791 | 20 μg | 2.9 (=stock solution) |

### Example 8

### Example 8.1—Production of Murine Interferon-γ (INFG) Constructs

Cloning and Production at evitria

Gene synthesis, cloning, transfection and harvest were outsourced to evitria AG (Schlieren, Switzerland). The corresponding cDNA was cloned into evitria's vector system using conventional (non-PCR based) cloning techniques. The evitria vector plasmid was gene synthesised. Plasmid DNA was prepared under low-endotoxin conditions based on anion exchange chromatography. DNA concentration was determined by measuring the absorption at a wavelength of 260 nm. Correctness of the sequences was verified with Sanger sequencing (with two sequencing reactions per plasmid.) Suspension-adapted CHO K1 cells (originally received from ATCC and adapted to serum-free growth in suspension culture at evitria) were used for production. The seed was grown in eviGrow medium, a chemically defined, animal-component free, serum-free medium. Cells were transfected with eviFect, evitria's custom-made, proprietary transfection reagent, and cells were grown after transfection in eviMake2, an animal-component free, serum-free medium. Supernatant was harvested by centrifugation and subsequent filtration (0.2 μm filter). Immediately after harvest Roche cOmplete™ Protease Inhibitor Cocktail was added at a concentration of 0.5 x.

### Protein Purification

The compound was purified from filtered cell culture supernatant referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralisation of the sample. The protein was concentrated by centrifugation (Millipore Amicon® ULTRA-15 (Art.Nr.: UFC903096), and aggregated protein was separated from monomeric protein by size exclusion chromatography (Superdex 200, GE Healthcare) in 20 mM histidine, 140 mM sodium chloride, pH 6.0. Monomeric compound fractions were pooled, concentrated (if required) using e.g., a MILLIPORE Amicon Ultra (30 MWCO) centrifugal concentrator, frozen and stored at −80° C. Part of the samples were provided for subsequent protein analytics and analytical characterization e.g. by CE-SDS, size exclusion chromatography (SE-HPLC) and mass spectrometry (LC-MS).

### Composition Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analysed by CE-SDS in the presence and absence of a reducing agent using a LabChipGXII or LabChip GX Touch (Perkin Elmer) (Perkin Elmer) with or without prior treatment with rapidPNGase F according to manufacturer's protocol. Determination of the aggregate content was performed by HPLC chromatography at 25° C. using analytical size-exclusion chromatography (TSKgel G3000 SW XL or UP-SW3000 columns) equilibrated in running buffer (200 mM KH₂PO₄, 250 mM KCl pH 6.2, 0.02% NaN3).

### Mass Determination by ESI-MS

For determination of the intact mass 25 ug (12.5 ug) per sample were diluted 1:4 (v/v) with “non-reducing” buffer contained in the rapidPNGase F enzyme kit (Rapid PNGaseF non-reducing, NEB #P0711S, Lot. 10085472, 10/21) and denatured for 2 minutes at 80° C. Subsequently, 0.3 μL rapidPNGase F (“non-reducing”) were added and the compound was deglycosylated for 10 minutes at 50° C. Thereafter, the sample was diluted with double distilled water to a final volume of 62.5 μL (31.25 μL). For determination of the mass of the reduced chains, 25 ug (12.5 ug) per sample were diluted with 1/4 (v/v) “reducing” buffer contained in the rapidPNGase F enzyme kit (Rapid PNGaseF reducing, NEB #P0710S, Lot. 10079163 07/21) and denatured for 2 minutes at 80° C. Subsequently, 0.3 μL rapidPNGase F (“reducing”) was added and the compound was deglycosylated for 10 minutes at 50° C. Thereafter, the sample was diluted with double distilled water to a final volume of 62.5 μL (31.25 μL). The sample were desalted by reversed phase chromatography on a C4 column (Acquity BEH300 C4, 1 mm 50 mm, 1.7 μm Charge 133380461; 150 μL/min, 75° C., 1.6 ug on column) and mass spectra were recorded using a QTOF type mass spectrometer (MAXIS, Bruker Daltonics). The mass spectrometer was calibrated before each sample sequence and lock mass correction was applied to obtain high mass accuracy. Data analysis was performed by summing up the mass spectra of chromatographic peaks and deconvoluting them with MaxEnt. Identity and integrity are examined by comparing the experimental masses with theoretical masses.

### Production and Purification of Murine Surrogate Molecule in Mask-Release Format

To create the masked format, murine IFNG was fused via a 30 amino acid linker (linker 1) to the C-terminus of murine IgG1 heavy chain. The IgG1 contained the FAP-binder (28H1, WO 2012/020006 A2) and an Fc containing the DAPG mutation. A scFv-based mask specific for murine IFNG was fused to the C-terminus of murine IFNG via a 30 amino acid linker (linker 2) containing the PQARK sequence. The compound was expressed using the transient CHO expression system at evitria. The compound was captured via MabSelectSure HP and eluted with a pH gradient to pH 3.0. Fractions were neutralised and analysed for composition by CE-SDS and HMW content by SE-HPLC. Fractions with the highest monomer content were pooled and further purified by preparative SEC. Fractions with the highest monomer content were pooled as the final batch. Composition analysis of the final batch revealed >95% monomer content by SE-HPLC and CE-SDS and <5% HMW content by SE-HPLC. Analysis by LC-MS confirmed sequence identity and sample purity.

TABLE 9

| Name | aa sequence | SEQ ID NO |
|---|---|---|
| P1AG3766 chain A | EIVLTQSPGTLSLSPGERATLSCRAS | 99 |
| (ID: C01676125) | QSVSRSYLAWYQQKPGQAPRLLIIG | |
| | ASTRATGIPDRFSGSGSGTDFTLTISR | |
| | LEPEDFAVYYCQQGQVIPPTFGQGT | |
| | KVEIKRADAAPTVSIFPPSSEQLTSG | |
| | GASVVCFLNNFYPKDINVKWKIDGS | |
| | ERQNGVLNSWTDQDSKDSTYSMSS | |
| | TLTLTKDEYERHNSYTCEATHKTST | |
| | SPIVKSFNRNEC | |
| P1AG3766 chain S (ID: | EVQLLESGGGLVQPGGSLRLSCAAS | 100 |
| C2184235346) | GFTFSSHAMSWVRQAPGKGLEWVS | |
| | AIWASGEQYYADSVKGRFTISRDNS | |
| | KNTLYLQMNSLRAEDTAVYYCAKG | |
| | WLGNFDYWGQGTLVTVSSAKTTPP | |
| | SVYPLAPGSAAQTNSMVTLGCLVK | |
| | GYFPEPVTVTWNSGSLSSGVHTFPA | |
| | VLQSDLYTLSSSVTVPSSTWPSQTVT | |
| | CNVAHPASSTKVDKKIVPRDCGCKP | |
| | CICTVPEVSSVFIFPPKPKDVLTITLT | |
| | PKVTCVVVAISKDDPEVQFSWFVDD | |
| | VEVHTAQTKPREEQINSTFRSVSELP | |
| | IMHQDWLNGKEFKCRVNSAAFGAP | |
| | IEKTISKTKGRPKAPQVYTIPPPKEQ | |
| | MAKDKVSLTCMITNFFPEDITVEWQ | |
| | WNGQPAENYKNTQPIMDTDGSYFV | |
| | YSKLNVQKSNWEAGNTFTCSVLHE | |
| | GLHNHHTEKSLSHSPGKGGGGSGG | |
| | GGSGGGGSGGGGSGGGGSGGGGG | |
| | HGTVIESLESLNNYFNSSGIDVEEKS | |
| | LFLDIWRNWQKDGDMKILQSQIISF | |
| | YLRLFEVLKDNQAISNNISVIESHLIT | |
| | TFFSNSKAKKDAFMSIAKFEVNNPQ | |
| | VQRQAFNELIRVVHQLLPESSLRKR | |
| | KRPGGGGSGGGGSGGGPQARKGGG | |
| | GGGSGGGGGQVQLQESGPGLVQAS | |
| | QPLSLTCTVSGFSLTTNSVHWIRQPA | |
| | GKCLEWMGAVWTDGTTDYNSALK | |
| | SRLTISRDTSKSQVFLEMSSLQTEDV | |
| | ATYYCAREHVYYGLVGWGQGTLV | |
| | TVSAGGGGSGGGGSGGGGSNIVMT | |
| | QSPKSMSISVGDRVTMNCKASQNV | |
| | GNNIAWYQQNPGQSPKLLIYYASNR | |
| | FTGVPDRFTGGGYGTDFTLTINNVQ | |
| | TEDAASYYCQRIFNAPNTFGCGTKL | |
| | ELK | |
| Linker 1 | GGGGSGGGGSGGGGSGGGGSGGG | 101 |
| | GSGGGGG | |
| Linker 2 | GGGGSGGGGSGGGPQARKGGGGG | 102 |
| | GSGGGGG | |
| Murine IFNG | HGTVIESLESLNNYFNSSGIDVEEKS | 103 |
| | LFLDIWRNWQKDGDMKILQSQIISF | |
| | YLRLFEVLKDNQAISNNISVIESHLIT | |
| | TFFSNSKAKKDAFMSIAKFEVNNPQ | |
| | VQRQAFNELIRVVHQLLPESSLRKR | |
| | KRP | |
| scFv mask | QVQLQESGPGLVQASQPLSLTCTVS | 104 |
| | GFSLTTNSVHWIRQPAGKCLEWMG | |
| | AVWTDGTTDYNSALKSRLTISRDTS | |
| | KSQVFLEMSSLQTEDVATYYCARE | |
| | HVYYGLVGWGQGTLVTVSAGGGG | |
| | SGGGGSGGGGSNIVMTQSPKSMSIS | |
| | VGDRVTMNCKASQNVGNNIAWYQ | |
| | QNPGQSPKLLIYYASNRFTGVPDRF | |
| | TGGGYGTDFTLTINNVQTEDAASYY | |
| | CQRIFNAPNTFGCGTKLELK | |

### Example 8.2—Activity Assay with Murine Interferon-γ Constructs

Interferon-Response Based on MHC1 and PDL1 Induction Upon Treatment with Masked FAP-IFNg Constructs after Digestion with Matriptase

The induction of MHC-I and PDL1 on murine MC38-huCEA tumor cell lines was assessed in response to treatment for two days with XMG1.2 scFv masked FAP-IFNg constructs containing one PQARK linker and compared to the activity of unmasked FAP-IFNg.

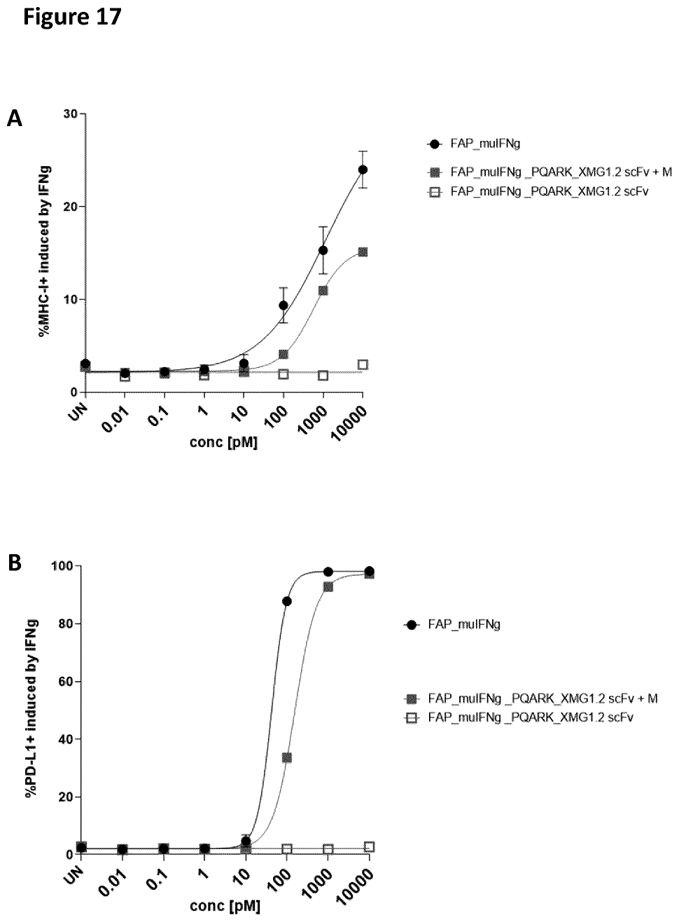

The constructs were incubated for two hours at 37° C. with recombinant matriptase prior to treatment. The FAP-IFNg XMG1.2 scFv-masked PQARK constructs induced MHC1 and PDL1 in tumor cell lines when the PQARK linker was digested with Matriptase. In contrast, the FAP-IFNg XMG1.2 scFv-masked construct without pre-incubation with recombinant matriptase did not induce MHC-I or PDL1 upregulation (FIGS. 17A and 17B).

### Material and Methods

MC38-huCEA cells were cultured in DMEM 10% FCS and harvested using cell dissociation buffer. Cells were washed in DMEM 10% FCS, resuspended in DMEM10% FCS, followed by assessment of cell viability and cell numbers using Eve cell counter. Cells were diluted to a concentration of 50,000/ml in DMEM 10% FCS and 100 μL of this cell suspension was seeded in cell culture treated 96F-well plates. Cells were incubated overnight at 37° C., 5% CO2 to ensure adherence of cells. Selected concentration of FAP-IFNg constructs were digested with or without 163 nM/4.4 ng recombinant matriptase (4735-SE, lot RIK071951, 0.44 mg/ml) at 37 C for two hours in matriptase buffer (50 mM Tris, 50 mM NaCl, 0.01% Tween 20, pH 9.0). After incubation, digested cytokine Fc-fusion solutions were diluted with DMEM 10% FCS to a concentration of 30 nM, and 50 μL were added per well of pre-seeded cells in 100 μL DMEM 10% FCS, rendering the final maximal concentration 10 nM per well. Cytokine Fc-fusion solutions were serially diluted in a ratio 1:10 until a final minimal concentration of 0.1 pM per well. The cells were incubated for 48h in the incubator.

After 48h, cells were washed with PBS, followed by a 10 minute incubation with 50 μL Trypsin EDTA. Detached cells were harvested in DMEM10% FCS and transferred in a round bottom 96 well-plate. Cells were centrifuged (500 g, 2 min), supernatant was discarded and 150 μL PBS was added per well followed by centrifugation (500 g, 2 min). Cells were resuspended in 50 μL staining mix containing Zombie Near IR fixable Viability Dye (Invitrogen, L10119). Afterwards cells were washed with FACS buffer and 50 μL antibody staining mix containing anti-muH-2Kb/H-2D-PE (BioLegend, 114608) and anti-muCD274-APC (BioLegend, 124312) was added for 20 min at 4 C. Thereafter, cells were washed with PBS and resuspended in 100 μL PFA and incubated for 25 min at RT. Afterwards, Cells were washed with 100 μL FACSbuffer, resuspended in 100 μL FCS buffer and measured on a BD FACS Canto.

### Example 9

### Determination of Matriptase Cleavage Rates Using SPR

Cleavage rates by recombinant Matriptase were investigated using surface plasmon resonance (SPR) on a Biacore T200 instrument (Cytiva). Biotinylated CD38 was immobilized on a Series S Sensorchip SA (Cytiva, 29104992) with final surface densities of 2000-4000 resonance units (RU). Protease-activatable antiFolR1-antiCD3 T cell bispecific antibodies (FOLR1 proTCBs) comprising a mask blocking the anti-CD3 antigen binding moiety and joined thereto by the respective protease-cleavable linker were used as a model system to assess Matripase cleavage rate. The proTCBs at a concentration of 10 nM were incubated with 50 pM of recombinant matriptase (R&D systems, 3946-SE) at 37° C. in PBS-T pH 7.4 as well as in PBS-T pH 6.5. The CD3& binding response and therefore the proTCB activation rate was monitored by continuously injecting the proTCB/matriptase mixtures for 30s at a flow rate of 5 l/min onto the surface for up to 10 hours. After each injection, the CD3e surface was regenerated by a 60s injection of 10 mM Glycine pH 1.5 at a flow rate of 5 μl/min. Within the same experiment, a concentration series of 0.16, 0.31, 0.63, 1.25 and 2.5 nM FOLR1 proTCB was injected to generate a calibration line and convert the obtained binding response of the proTCBs from resonance units (RU) to molar concentrations (nM). Molar concentrations of the activated proTCBs were plotted against the incubation time and cleavage rates (pM/min) were calculated by determining the slopes of each derived straight line. The results are provided in Table 10.

TABLE 10
Initial cleavage rate at different pH.

Initial Rate of Reaction (pM/min)

| Concept ID/recognition site | pH 7.4 | pH 6.5 |
|---|---|---|
| P1AF5420/PQARK | 5.5 | 1.3 |
| P1AF5419/HQARK | 3.0 | 1.4 |
| P1AF1596/PMAKK | 1.8 | 0.4 |

TABLE 11
Exemplary Sequences of FOLR1 TCBs tested in Example 10 and cleavable linkers.

| Name | aa sequence | SEQ ID NO |
|---|---|---|
| FOLR1 | QAVVTQEPSLTVSPGGTVTLTCGSSTGAVTTSNYANWVQ | 105 |
| proTCB | EKPGQAFRGLIGGTNKRAPGTPARFSGSLLGGKAALTLS | |
| common light | GAQPEDEAEYYCALWYSNLWVFGGGTKLTVLGQPKAA | |
| chain | PSVTLFPPSSEELQANKATLVCLISDFYPGAVTVAWKADS | |
| (presenet in | SPVKAGVETTTPSKQSNNKYAASSYLSLTPEQWKSHRSY | |
| P1AF1596, | SCQVTHEGSTVEKTVAPTECS | |
| P1AF5419, | | |
| P1AF5420) | | |
| FOLR1 | EVQLVESGGGLVKPGGSLRLSCAASGFTFSNAWMSWVR | 106 |
| proTCB | QAPGKGLEWVGRIKSKTDGGTTDYAAPVKGRFTISRDDS | |
| heavy chain 1 | KNTLYLQMNSLKTEDTAVYYCTTPWEWSWYDYWGQG | |
| (present in | TLVTVSSASTKGPSVFPLAPSSKSTSGGTAALGCLVKDYF | |
| P1AF1596, | PEPVTVSWNSGALTSGVHTFPAVLQSSGLYSLSSVVTVPS | |
| P1AF5419, | SSLGTQTYICNVNHKPSNTKVDKKVEPKSCDKTHTCPPC | |
| P1AF5420) | PAPEAAGGPSVFLFPPKPKDTLMISRTPEVTCVVVDVSHE | |
| | DPEVKFNWYVDGVEVHNAKTKPREEQYNSTYRVVSVLT | |
| | VLHQDWLNGKEYKCKVSNKALGAPIEKTISKAKGQPREP | |
| | QVCTLPPSRDELTKNQVSLSCAVKGFYPSDIAVEWESNG | |
| | QPENNYKTTPPVLDSDGSFFLVSKLTVDKSRWQQGNVFS | |
| | CSVMHEALHNHYTQKSLSLSPGK | |
| FOLR1 | QIQLVQSGPELKKPGETVKISCKASGYTVTDYSMNWVKQ | 107 |
| proTCB | APGKCLKWMGWINTETGEPRYTDDFKGRFAFSLETSAST | |
| PMAKK | AYLQINNLKNEDSATYFCAREGDYDVFDYWGHGTTLKV | |
| heavy chain 2 | SSGGGGSGGGGSGGGGSGGGGSDIVLTQSPASLAVSLGQ | |
| (present in | RATISCRASKSVSTSSYSYMHWYQQKPGQPPKLLIKYVS | |
| P1AF1596) | YLESGVPARFSGSGSGTDFTLNIHPVEEEDAATYYCQHSR | |
| | EFPYTFGCGTKLEIKSGGGSGGGGSPMAKKGGGGSGGG | |
| | GSGGGGSGGSEVQLLESGGGLVQPGGSLRLSCAASGFTF | |
| | SSYAMNWVRQAPGKGLEWVSRIRSKYNNYATYYADSV | |
| | KGRFTISRDDSKNTLYLQMNSLRAEDTAVYYCVRASNFP | |
| | ASYVSYFAYWGQGTLVTVSSASTKGPSVFPLAPSSKSTS | |
| | GGTAALGCLVKDYFPEPVTVSWNSGALTSGVHTFPAVL | |
| | QSSGLYSLSSVVTVPSSSLGTQTYICNVNHKPSNTKVDKK | |
| | VEPKSCDGGGGSGGGGSEVQLVESGGGLVKPGGSLRLSC | |
| | AASGFTFSNAWMSWVRQAPGKGLEWVGRIKSKTDGGTT | |
| | DYAAPVKGRFTISRDDSKNTLYLQMNSLKTEDTAVYYCT | |
| | TPWEWSWYDYWGQGTLVTVSSASTKGPSVFPLAPSSKS | |
| | TSGGTAALGCLVKDYFPEPVTVSWNSGALTSGVHTFPAV | |
| | LQSSGLYSLSSVVTVPSSSLGTQTYICNVNHKPSNTKVDK | |
| | KVEPKSCDKTHTCPPCPAPEAAGGPSVFLFPPKPKDTLMI | |
| | SRTPEVTCVVVDVSHEDPEVKFNWYVDGVEVHNAKTKP | |
| | REEQYNSTYRVVSVLTVLHQDWLNGKEYKCKVSNKAL | |
| | GAPIEKTISKAKGQPREPQVYTLPPCRDELTKNQVSLWCL | |
| | VKGFYPSDIAVEWESNGQPENNYKTTPPVLDSDGSFFLY | |
| | SKLTVDKSRWQQGNVFSCSVMHEALHNHYTQKSLSLSP | |
| | GK | |
| FOLR1 | QIQLVQSGPELKKPGETVKISCKASGYTVTDYSMNWVKQ | 108 |
| proTCB | APGKCLKWMGWINTETGEPRYTDDFKGRFAFSLETSAST | |
| HQARK | AYLQINNLKNEDSATYFCAREGDYDVFDYWGHGTTLKV | |
| heavy chain 2 | SSGGGGSGGGGSGGGGSGGGGSDIVLTQSPASLAVSLGQ | |
| (present in | RATISCRASKSVSTSSYSYMHWYQQKPGQPPKLLIKYVS | |
| P1AF5419) | YLESGVPARFSGSGSGTDFTLNIHPVEEEDAATYYCQHSR | |
| | EFPYTFGCGTKLEIKSGGGSGGGGSHQARKGGGGSGGG | |
| | GSGGGGSGGSEVQLLESGGGLVQPGGSLRLSCAASGFTF | |
| | SSYAMNWVRQAPGKGLEWVSRIRSKYNNYATYYADSV | |
| | KGRFTISRDDSKNTLYLQMNSLRAEDTAVYYCVRASNFP | |
| | ASYVSYFAYWGQGTLVTVSSASTKGPSVFPLAPSSKSTS | |
| | GGTAALGCLVKDYFPEPVTVSWNSGALTSGVHTFPAVL | |
| | QSSGLYSLSSVVTVPSSSLGTQTYICNVNHKPSNTKVDKK | |
| | VEPKSCDGGGGSGGGGSEVQLVESGGGLVKPGGSLRLSC | |
| | AASGFTFSNAWMSWVRQAPGKGLEWVGRIKSKTDGGTT | |
| | DYAAPVKGRFTISRDDSKNTLYLQMNSLKTEDTAVYYCT | |
| | TPWEWSWYDYWGQGTLVTVSSASTKGPSVFPLAPSSKS | |
| | TSGGTAALGCLVKDYFPEPVTVSWNSGALTSGVHTFPAV | |
| | LQSSGLYSLSSVVTVPSSSLGTQTYICNVNHKPSNTKVDK | |
| | KVEPKSCDKTHTCPPCPAPEAAGGPSVFLFPPKPKDTLMI | |
| | SRTPEVTCVVVDVSHEDPEVKFNWYVDGVEVHNAKTKP | |
| | REEQYNSTYRVVSVLTVLHQDWLNGKEYKCKVSNKAL | |
| | GAPIEKTISKAKGQPREPQVYTLPPCRDELTKNQVSLWCL | |
| | VKGFYPSDIAVEWESNGQPENNYKTTPPVLDSDGSFFLY | |
| | SKLTVDKSRWQQGNVFSCSVMHEALHNHYTQKSLSLSP | |
| | GK | |
| FOLR1 | QIQLVQSGPELKKPGETVKISCKASGYTVTDYSMNWVKQ | 109 |
| proTCB | APGKCLKWMGWINTETGEPRYTDDFKGRFAFSLETSAST | |
| PQARK | AYLQINNLKNEDSATYFCAREGDYDVFDYWGHGTTLKV | |
| heavy chain 2 | SSGGGGSGGGGSGGGGSGGGGSDIVLTQSPASLAVSLGQ | |
| (present in | RATISCRASKSVSTSSYSYMHWYQQKPGQPPKLLIKYVS | |
| P1AF5420) | YLESGVPARFSGSGSGTDFTLNIHPVEEEDAATYYCQHSR | |
| | EFPYTFGCGTKLEIKSGGGSGGGGSPQARKGGGGSGGGG | |
| | SGGGGSGGSEVQLLESGGGLVQPGGSLRLSCAASGFTFSS | |
| | YAMNWVRQAPGKGLEWVSRIRSKYNNYATYYADSVKG | |
| | RFTISRDDSKNTLYLQMNSLRAEDTAVYYCVRASNFPAS | |
| | YVSYFAYWGQGTLVTVSSASTKGPSVFPLAPSSKSTSGG | |
| | TAALGCLVKDYFPEPVTVSWNSGALTSGVHTFPAVLQSS | |
| | GLYSLSSVVTVPSSSLGTQTYICNVNHKPSNTKVDKKVEP | |
| | KSCDGGGGSGGGGSEVQLVESGGGLVKPGGSLRLSCAA | |
| | SGFTFSNAWMSWVRQAPGKGLEWVGRIKSKTDGGTTDY | |
| | AAPVKGRFTISRDDSKNTLYLQMNSLKTEDTAVYYCTTP | |
| | WEWSWYDYWGQGTLVTVSSASTKGPSVFPLAPSSKSTS | |
| | GGTAALGCLVKDYFPEPVTVSWNSGALTSGVHTFPAVL | |
| | QSSGLYSLSSVVTVPSSSLGTQTYICNVNHKPSNTKVDKK | |
| | VEPKSCDKTHTCPPCPAPEAAGGPSVFLFPPKPKDTLMIS | |
| | RTPEVTCVVVDVSHEDPEVKFNWYVDGVEVHNAKTKPR | |
| | EEQYNSTYRVVSVLTVLHQDWLNGKEYKCKVSNKALG | |
| | APIEKTISKAKGQPREPQVYTLPPCRDELTKNQVSLWCLV | |
| | KGFYPSDIAVEWESNGQPENNYKTTPPVLDSDGSFFLYS | |
| | KLTVDKSRWQQGNVFSCSVMHEALHNHYTQKSLSLSPG | |
| | K | |
| PQARK linker | GGGGSGGGGSPQARKGGGGSGGGGSGGGGSGGS | 110 |
| HQARK | GGGGSGGGGSHQARKGGGGSGGGGSGGGGSGGS | 111 |
| linker | | |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An isolated polypeptide comprising a protease recognition site, wherein the protease recognition site is a substrate for matriptrase and comprises or consists of the sequence PQARK according to SEQ ID NO: 32 or HQARK according to SEQ ID NO: 33.

2. The isolated polypeptide according to claim 1, comprising one or more unstructured linker comprising the protease recognition site.

3. The isolated polypeptide according to claim 2, wherein the one or more unstructured linker does not exhibit a secondary structure.

4. The isolated polypeptide according to claim 1, wherein the protease recognition site is part of a cleavable moiety (CM).

5. The isolated polypeptide according to claim 4, wherein the isolated polypeptide comprises at least one moiety (M) selected from the group consisting of a moiety that is located amino (N) terminally to the CM (MN), a moiety that is located carboxyl (C) terminally to the CM (MC), and combinations thereof, and wherein the MN or MC is selected from the group consisting of an antibody or antigen binding fragment thereof (AB), a therapeutic agent, an antineoplastic agent, a toxic agent, a drug, and a detectable label.

6. The isolated polypeptide according to claim 1, comprising a sequence selected from the group consisting of GGGGSGGGGSGGGPQARKGGGGGGSGGGGG according to SEQ ID NO: 102, GGGGSGGGGSPQARKGGGGSGGGGSGGGGSGGS according to SEQ ID NO: 110 and GGGGSGGGGSHQARKGGGGSGGGGSGGGGSGGS according to SEQ ID NO: 111.

7. A therapeutic agent, comprising a protease recognition site, wherein the protease recognition site is PQARK according to SEQ ID NO: 32 or HQARK according to SEQ ID NO: 33.

8. The therapeutic agent according to claim 7, wherein the therapeutic agent is an isolated polypeptide.

9. The therapeutic agent according to claim 7, wherein the therapeutic agent is a cancer treatment.

10. (canceled)

11. An isolated polynucleotide encoding the isolated polypeptide of claim 1.

12. An expression vector, comprising the polynucleotide of claim 11.

13. A host cell, comprising the polynucleotide of claim 11.

14. A method of producing a polypeptide, comprising culturing the host cell of claim 13 under conditions suitable for the expression of the polypeptide.

15. An isolated polypeptide produced by the method of claim 14.

16. A pharmaceutical composition, comprising the isolated polypeptide of claim 1 and a pharmaceutically acceptable carrier.

17. The isolated peptide of claim 1, for use in the treatment of a disease in an individual in need thereof.

18. The isolated polypeptide of claim 17, wherein said disease is cancer.

19. (canceled)

20. A method of treating disease in an individual, comprising administering to said individual a therapeutically effective amount of a composition comprising the isolated polypeptide of claim 1 in a pharmaceutically acceptable form.

21. The method of claim 20, wherein said disease is cancer.

22. (canceled)

23. The isolated polypeptide according to claim 4, comprising a sequence selected from the group consisting of SEQ ID NOs 71, 73, 75, 76, 78, 80, and 82.
